(19) United States
(12) Patent Application Publication
(10) Pub. No.: US 2023/0375531 A1
(43) Pub. Date: Nov. 23, 2023

(54) A Method Of Detecting an Analyte and Related Systems

(71) Applicant: Massachusetts Institute of Technology, Cambridge, MA (US)

(72) Inventors: Patthara Kongsuphol, Singapore (SG); Huan Jia, Singapore (SG); Hadley D. Sikes, Cambridge, MA (US)

(21) Appl. No.: 18/044,237

(22) PCT Filed: Sep. 17, 2021
(86) PCT No.: PCT/IB2021/058484

§ 371 Date: Mar. 7, 2023

(30) Foreign Application Priority Data
Sep. 18, 2020 (SG) 10202009197S

(51) Int. Cl. G01N 33/52; G01N 33/543

(52) U.S. Cl. CPC G01N 33/526; G01N 33/54388; G01N 2333/165; G01N 2469/20

(57) ABSTRACT

There is provided a method of detecting an analyte in a sample, the method comprising: incubating said sample with a reporter agent to allow said reporter agent to bind to said analyte, if present, in said sample; applying the incubated sample to a cellulose substrate to allow said analyte that is bound to said reporter agent to be captured onto said cellulose substrate by a capture agent comprising a cellulose binding domain (CBD), wherein said capture agent is: (i) incubated with said sample prior to the applying step; and/or (ii) immobilised on said cellulose substrate prior to the applying step; and detecting a signal effected by the reporter agent to determine the presence or absence of said analyte. There is also provided related systems and methods of identifying an infection and detecting an antibody against an infection in a subject, in particular SARS-Cov-2 via lateral flow or vertical flow assays.

### TECHNICAL FIELD

The present disclosure relates broadly to a method of detecting an analyte, and related methods and systems. Also provided are related methods of identifying an infection and detecting an antibody against an infection in a subject.

### BACKGROUND

Cellulose substrate offers low production cost and ease of scalability. Therefore, it is often used as a substrate for diagnostic platforms such as rapid diagnostic tests (RDTs). In a conventional RDT, reporter agents (e.g. labelled antibodies) are dry stored on the cellulose substrate and capture agents (e.g. antibodies) are immobilized to the test zone prior to performing the assay. The target analytes are relayed to the reporter agents and the immobilized capture agents by the capillary force. The analytes have only less than 10 seconds to interact with the reporter agents while the capture agents have only a few seconds to capture the analytes-reporter agent complex before they flow pass the test zone. Due to the limited resident/interaction time of the target analytes to form a complex with the reporter and capture agents, the conventional RDT suffers from low sensitivity and false negative results.

Thus, there is a need to provide a method of detecting an analyte, and related methods and systems that address or at least ameliorate the above-mentioned problems.

### SUMMARY

In one aspect, there is provided a method of detecting an analyte in a sample, the method comprising: applying the sample to a cellulose substrate to allow said analyte, if present in said sample, to be captured onto said cellulose substrate by a capture agent comprising a cellulose binding domain (CBD), wherein said capture agent is: (i) incubated with said sample prior to the applying step; and/or (ii) immobilised on said cellulose substrate prior to the applying step; and determining a presence or absence of said analyte captured on said cellulose substrate by said capture agent.

In one embodiment, determining a presence or absence of said analyte captured on the cellulose substrate by said capture agent comprises detecting a signal effected by a reporter agent on the cellulose substrate, wherein said reporter agent: (i) comprises an analyte binder and said reporter agent is contacted with the sample to allow said reporter agent to bind to said analyte, if present, in said sample; or (ii) comprises a competing binder having affinity for said capture agent and said reporter agent is contacted with said capture agent to allow said reporter agent to bind to said capture agent.

In one embodiment, the amount of said capture agent is not more than 1000-fold molar excess, optionally not more than 100-fold molar excess, further optionally not more than 60-fold molar excess of said analyte.

In one embodiment, where said reporter agent comprises an analyte binder, the method comprises incubating the reporter agent and the capture agent with said sample prior to the applying step and said analyte, when present, is part of a reporter agent-analyte-capture agent complex prior to the applying step.

In one embodiment, where said reporter agent comprises an analyte binder, the method comprises incubating the reporter agent with said sample and immobilizing said capture agent on the cellulose substrate prior to the applying step and said analyte when present, is bound to the reporter agent to form a reporter agent-analyte complex prior to the applying step.

In one embodiment, where said reporter agent comprises a competing binder, the method comprises incubating said capture agent with said reporter agent and/or said sample prior to the applying step to form a reporter agent-capture agent complex and/or an analyte-capture agent complex prior to the applying step.

In one embodiment, where said reporter agent comprises a competing binder, the method comprises incubating said reporter agent with said sample and immobilizing said capture agent on said cellulose substrate prior to the applying step and dispensing the reporter agent incubated with said sample on said cellulose substrate after the applying step.

In one embodiment, a plurality of capture agents is immobilised on said cellulose substrate in a substantially uniform orientation.

In one embodiment, in said capture agent, said CBD is coupled to a C-terminus of a binding protein for said analyte.

In one embodiment, the presence or absence of said analyte in said sample is determined within 15 minutes from the start of the incubating step.

In one embodiment, said method is a method of detecting coronavirus in a sample.

In one embodiment, said method is a lateral flow assay method.

In one embodiment, said method is a vertical flow assay method and the applying step comprises allowing the sample to flow through a plurality of cellulose substrate layers.

In one aspect, there is provided a method of detecting an antibody against SARS-CoV-2 in a subject, the method comprising: applying a sample from the subject to a cellulose substrate to allow said antibody, if present in said sample, to be captured onto said cellulose substrate by a capture agent comprising a cellulose binding domain (CBD), wherein said capture agent is: (i) incubated with said sample prior to the applying step; and/or (ii) immobilised on said cellulose substrate prior to the applying step; contacting the capture agent with a reporter agent to allow said reporter agent to bind to said capture agent, wherein the reporter agent comprises a competing binder having affinity for said capture agent; and detecting a signal effected by the reporter agent to determine a presence or absence of said antibody captured on said cellulose substrate by said capture agent.

In one embodiment, the sample is selected from the group consisting of: a plasma sample, a serum sample and a whole blood sample.

In one embodiment, said capture agent is incubated with said sample before said capture agent is contacted with said reporter agent.

In one aspect, there is provided a method of identifying SARS-CoV-2 infection in a subject, the method comprising: incubating a sample from the subject with a reporter agent to allow said reporter agent to bind to a SARS-CoV-2 protein, if present, in said sample; applying the incubated sample to a cellulose substrate to allow the SARS-CoV-2 protein that is bound to said reporter agent to be captured onto said cellulose substrate by a capture agent comprising a cellulose binding domain (CBD), wherein said capture agent is: (i) incubated with said sample prior to the applying step; and/or (ii) immobilised on said cellulose substrate prior to the applying step; and detecting a signal effected by the reporter agent to determine a presence or absence of said SARS-CoV-2 protein captured on said cellulose substrate by said capture agent.

In one embodiment, the SARS-CoV-2 protein comprises SARS-CoV-2 nucleocapsid (N) protein.

In one embodiment, said reporter agent and said capture agent are configured to bind to different epitopes of the SARS-CoV-2 protein.

In one embodiment, said sample is selected from the group consisting of: a saliva sample, a sputum sample, a nasal fluid sample, a pharyngeal fluid sample, a nasopharyngeal fluid sample and an oropharyngeal fluid sample.

### Definitions

The terms “contacting”, “contacted” and the like as used herein in relation to two or more substances broadly include conditions which allow contact or interaction between the two or more substances whether in solution or in solid phase. For example, contacting two or more substances may comprise mixing together the two or more substances in solution or adding the two or more substances to the same substrate. The terms do not necessarily require actual physical contact between the two or more substances. The terms “incubating”, “incubated” and the like as used herein in relation to two or more substances means contacting two or more substances for a sufficient duration of time to allow interaction between the two or more substances to take place. No specific temperature requirements are implied by the terms “incubating”, “incubated” and the like unless otherwise indicated.

The terms “bind,” “binding,”, “bound” and the like as used herein broadly include to any type of immobilization, formation of a complex, fixation, or attachment between two or more substances, between a substance and one or more surfaces, or a combination thereof, regardless of the mechanism or mechanisms of attachment involved. Examples of mechanisms include, but are not limited to, formation of complexes (e.g. protein-protein complex, antibody-antigen complex, non-antibody protein-antigen complex etc.), covalent bonding, ionic bonding, electrostatic forces, hydrogen bonding, van der Walls attraction, hydrophobic effect, absorption and adsorption.

The term “affinity” as used herein refers to an attraction or non-random interaction between two molecules or parts thereof, for example, between cellulose and cellulose-binding domain.

The term “micro” when used as a unit prefix (e.g. 1 micro (μ)) generally denotes a factor of 10⁻⁶. When used in relation to dimensions, the term is to be interpreted broadly to include dimensions from about 1 micron to about 1000 microns.

The term “nano” when used as a unit prefix, (e.g. 1 nano (n)) generally denotes a factor of 10⁻⁹. When used in relation to dimensions, the term is to be interpreted broadly to include dimensions less than about 1000 nm.

The term “particle” as used herein broadly refers to a discrete entity or a discrete body. The particle described herein can include an organic, an inorganic or a biological particle. The particle used described herein may also be a macro-particle that is formed by an aggregate of a plurality of sub-particles or a fragment of a small object. The particle of the present disclosure may be spherical, substantially spherical, or non-spherical, such as irregularly shaped particles or ellipsoidally shaped particles. In some examples, the term “size” when used to refer to the particle broadly refers to the largest dimension of the particle. For example, when the particle is substantially spherical, the term “size” can refer to the diameter of the particle; or when the particle is substantially non-spherical, the term “size” can refer to the largest length of the particle. In some examples, the term “size” when used to refer to the particle broadly refers to the weight of the particle. For example, the “size” of a protein may be described using its molecular mass in kilodaltons (kDa) unit.

The terms “coupled” or “connected” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship.

For example, when element A is associated with element B, elements A and B may be directly or indirectly attached to each other or element A may contain element B or vice versa.

The term “adjacent” used herein when referring to two elements refers to one element being in close proximity to another element and may be but is not limited to the elements contacting each other or may further include the elements being separated by one or more further elements disposed therebetween.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. It is to be appreciated that the individual numerical values within the range also include integers, fractions and decimals.

Furthermore, whenever a range has been described, it is also intended that the range covers and teaches values of up to 2 additional decimal places or significant figures (where appropriate) from the shown numerical end points. For example, a description of a range of 1% to 5% is intended to have specifically disclosed the ranges 1.00% to 5.00% and also 1.0% to 5.0% and all their intermediate values (such as 1.01%, 1.02% . . . 4.98%, 4.99%, 5.00% and 1.1%, 1.2% . . . 4.8%, 4.9%, 5.0% etc.,) spanning the ranges. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure. Unless otherwise indicated, it will also be appreciated that a method and/or process disclosed herein is not limited to the steps being carried out sequentially or at different time, but also includes the steps or some of the steps being carried out simultaneously, concurrently or at the same time.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DESCRIPTION OF EMBODIMENTS

Exemplary, non-limiting embodiments of a method of detecting an analyte, and related methods and systems are disclosed hereinafter.

In various embodiments, there is provided a method of detecting an analyte, the method comprising capturing the analyte onto a cellulose substrate with a capture agent comprising a cellulose binding domain. In various embodiments, there is provided a method of detecting an analyte in a sample, the method comprising incubating the sample with a reporter agent and/or a capture agent before applying the sample to a substrate. In various embodiments, there is provided a method of detecting an analyte in a sample, the method comprising: applying the sample to a cellulose substrate to allow said analyte, if present in said sample, to be captured onto said cellulose substrate by a capture agent comprising a cellulose binding domain (CBD), wherein said capture agent is: (i) incubated with said sample prior to the applying step; and/or (ii) immobilised on said cellulose substrate prior to the applying step; and determining a presence or absence of said analyte captured on said cellulose substrate by said capture agent. Methods for determining whether an analyte is captured on a cellulose substrate that are known in the art may be suitably applied in the disclosed method. For example, a detectable reporter agent may be used to bind to the analyte or the capture agent on the cellulose substrate and the signal effected by the reporter agent may indicate the presence or absence of analyte on the cellulose substrate.

Detecting an analyte may comprise detecting a presence, an absence, an amount/concentration, or a relative amount/concentration of an analyte. The detection may be qualitative, quantitative or semi quantitative. For example, an amount/concentration or a relative amount/concentration of an analyte can be determined by measuring an intensity of the signal or a rate of production of the signal effected by the reporter agent, and then comparing the intensity or rate with that effected by a control sample or a comparative sample containing a known amount of analytes. If the intensity or rate of the signal obtained from the sample is greater than that obtained from the control sample or the comparative sample in a non-competitive binding assay, this may be indicative that the sample contains a greater amount/concentration of the analyte than the control sample or the comparative sample. The amount/concentration of the analyte in the sample may be determined by calculating the ratio of the intensity or rate obtained from the sample to the intensity or rate obtained from the control sample or the comparative sample.

The method of detecting an analyte may also comprise detecting two or more analytes, or a plurality of analytes. For example, two or more different analytes, or a plurality of different analytes, may be detected by use of different reporter agents, or by capturing the different analytes onto different spatial regions on the cellulose substrate.

The term “analyte”, “analyte of interest” or “target analyte” as used herein broadly refers to a substance which is to be detected/measured. In various embodiments, an “analyte” not only includes any substance for which there exists a naturally occurring binding member specific to the analyte, but also includes any substance for which a binding member specific to the analyte can be prepared e.g. an affitin. Examples of analytes include, but are not limited to, prokaryotic or eukaryotic cells of any type, microorganisms, bacteria, pathogens, viruses, prions, organic compounds, lipids, carbohydrates, hormones, antibodies, antigen-binding proteins, peptides, amino acids, nucleic acids/polynucleotides such as deoxyribonucleic acid (DNA) and ribonucleic acids (RNA), steroids, vitamins, drugs (including those administered for therapeutic purposes as well as those administered for illicit purposes), drug intermediates, toxins, chemicals, pesticides, pollutants, metal, heavy metal, metal ions and metabolites, portions, fragments and extracts of the aforementioned materials and combinations thereof. In some embodiments, the analyte comprises a virus. In some embodiments, the analyte comprises a viral protein. In some embodiments, the analyte, or portion thereof, is antigenic or haptenic having at least one determinant site, or is a member of a binding pair.

In some embodiments, the analyte comprises a biological material. In some embodiments, the analyte comprises a natural material, for example, a naturally occurring biological material. In some embodiments, the analyte comprises a synthetic material, for example, pesticides.

The sample (or a test sample) may be a sample suspected of containing an analyte. A sample suspected of containing an analyte may refer to a sample for which the content of the analyte is unknown or unconfirmed. For example, a sample from a human suspected of having a disease (and therefore suspected of having the disease-associated analyte in his/her sample), but not known to have the disease, may constitute a sample suspected of containing an analyte. Embodiments of the method may also be used for confirmatory or further testing of a sample (e.g. to confirm the presence or absence of an analyte in the sample, or to further determine an amount/relative amount of the analyte in the sample) that has already been evaluated earlier by another method. Embodiments of the method also provide for a control sample that may be used to obtain an indication on whether the method is correctly performed. A “control sample” is distinguished from the “sample” or “test sample” in that the contents of the analyte (presence, absence etc.) in the “control sample” is known. A “control sample” includes both negative and positive control samples.

The sample may be obtained from any source. For example, the sample may be a biological sample, a pharmaceutical sample, an environmental sample, a food sample etc. In some embodiments, the sample comprises a biological sample. Examples of biological samples include, but are not limited to blood, serum, plasma, sputum, saliva, lavage fluid (e.g. bronchial lavage fluid, alveolar lavage fluid and bronchoalveolar lavage fluid), nasal fluid/swab/wash/aspirate, anterior nares fluid/swab/wash/aspirate, nasal mid-turbinate fluid/swab/wash/aspirate, pharyngeal fluid/swab/wash/aspirate, nasopharyngeal fluid/swab/wash/aspirate, oropharyngeal fluid/swab/wash/aspirate, tissue biopsy e.g. lung biopsy, cerebrospinal fluid, urine, faeces, stool, anal swab, semen, sweat, tears, processed fractions thereof and the like. Saliva samples may be obtained, for example, from the mouth, the throat include the deep throat, the posterior oropharyngeal, the oropharynx or the naso-oropharyngeal etc.

In some embodiments, the method comprises obtaining a sample. In some embodiments, the method comprises processing a sample prior to its provision to the cellulose substrate. Methods of processing different types of samples known in the art may be employed prior to their provision to the substrate. For example, methods of processing a biological sample may involve centrifugation to separate a sample into different fractions, cell lysis, extraction of DNA and/or RNA, disintegration and/or dissolving of sample, purification and/or sample concentration. In one embodiment, the method further comprises substantially dissolving/solubilizing the sample in a solvent to obtain a liquid/fluid sample.

In some embodiments, where the analyte comprises a polynucleotide or a nucleic acid, preparing the sample comprises subjecting the sample to an amplification condition suitable for amplifying the nucleic acid analyte to a detectable level. In some embodiments, the analyte comprises a polynucleotide/nucleic acid, and the method further comprises subjecting the sample to an amplification reaction configured to amplify the polynucleotide/nucleic acid prior to the incubating step. Amplification reactions known in the art may be employed. The amplification reactions may include but are not limited to polymerase chain reaction (PCR), ligase chain reaction (LCR), loop mediated isothermal amplification (LAMP), nucleic acid sequence based amplification (NASBA), self-sustained sequence replication (3SR), rolling circle amplification (RCA) or any other process whereby one or more copies of a particular polynucleotide sequence or nucleic acid sequence may be generated from a polynucleotide template sequence or nucleic acid template sequence.

In various embodiments, the method further comprises, after the sample applying step but prior to the detecting determining/detecting step, washing the cellulose substrate with a washing reagent to remove free molecules from the cellulose substrate. Advantageously, this can improve the accuracy of the detection of signal effected by the reporter agent to determine the presence or absence of said analyte captured on said cellulose substrate by said capture agent. As unbounded and/or weakly bounded molecules (e.g., not containing the target analyte of interest) are removed, the likelihood of obtaining false positives (e.g., when the reporter agent is one that binds to an analyte) is also reduced.

The method may also further comprise, blocking the cellulose substrate with a blocking agent/buffer. The blocking step may be carried out prior to the sample applying step. For example, when the capture agent has been incubated with the sample prior to the sample applying step, the blocking step may be carried out prior to the sample applying step. For example, when the capture agent is immobilised on the one more or more test zones of the cellulose substrate prior to the sample applying step, the blocking step may be carried out after applying the capture agent to the test zones and before applying the sample. Advantageously, the blocking step may improve the specificity of an assay by reducing background interference and improving the signal-to-noise ratio.

Examples of blocking agents/buffers include, but are not limited to, bovine serum albumin (BSA) e.g., at from about 1 to about 5% concentration, non-fat dry milk (NFDM) e.g., at from about 0.1 to about 3% concentration, fish gelatin, whole sera, polyethylene glycol (PEG), polyvinyl alcohol (PVA), and polyvinylpyrrolidone (PVP).

The reporter agent may be any detectable agent that is capable of indicating/reflecting a presence/absence and/or an amount of analyte that is captured by the capture agent. For example, the reporter agent may bind to an analyte and the detection of a signal effected by the reporter agent on a cellulose substrate may indicate the presence of analyte that is captured by the capture agent on the cellulose substrate. For example, the reporter agent may also compete with an analyte for binding to a capture agent and detection of signal effected by the reporter agent on a cellulose substrate may indicate that little or no analyte is captured by the capture agent on the cellulose substrate. In various embodiments therefore, the reporter agent comprises an analyte binder or a competing binder.

In one embodiment, the reporter agent comprises an analyte binder or a binding protein that is capable of binding to the analyte. In various embodiments, the reporter agent/analyte binder is capable of recognising and/or binding to the analyte. In various embodiments, the reporter agent/analyte binder has binding affinity for the analyte. In various embodiments, the reporter agent/analyte binder is specific to the analyte. In various embodiments, the affinity between the reporter agent/analyte binder and the analyte is not more than about 10⁻⁶ M, not more than about 10⁻⁷ M, not more than about 10⁻⁸ M, not more than about 10⁻⁹ M, not more than about 10⁻¹⁰ M, not more than about 10⁻¹¹ M, not more than about 10⁻¹² M, not more than about 10⁻¹³ M, not more than about 10⁻¹⁴ M, not more than about 10⁻¹⁵ M or less than about 10⁻¹⁵ M. In some embodiments, the affinity between the reporter agent/analyte binder and the analyte is in the micromolar range, optionally in the low micromolar range. In some embodiments, the affinity between the reporter agent/analyte binder and the analyte is in the nanomolar range, optionally in the low nanomolar range. In some embodiments, the affinity between the reporter agent/analyte binder and the analyte is in the picomolar range, optionally in the low picomolar range. In some embodiments, the affinity between the reporter agent/analyte binder and the analyte is in the femtomolar range, optionally in the low femtomolar range.

In one embodiment, the reporter agent comprises a competing binder or a binding protein that is capable of binding to the capture agent. In various embodiments, the reporter agent/competing binder is capable of recognising and/or binding to the capture agent. In various embodiments, the reporter agent/competing binder has binding affinity for the capture agent. In various embodiments, the reporter agent/competing binder is specific to the capture agent. In various embodiments, the affinity between the reporter agent/competing binder and the capture agent is not more than about 10⁻⁶ M, not more than about 10⁻⁷ M, not more than about 10⁻⁸ M, not more than about 10⁻⁹ M, not more than 1.0 about 10⁻¹⁰ M, not more than about 10⁻¹¹ M, not more than about 10⁻¹² M, not more than about 10⁻¹³ M, not more than about 10⁻¹⁴ M, not more than about 10⁻¹⁵ M or less than about 10⁻¹⁵ M. In some embodiments, the affinity between the reporter agent/competing binder and the capture agent is in the micromolar range, optionally in the low micromolar range. In some embodiments, the affinity between the reporter agent/competing binder and the capture agent is in the nanomolar range, optionally in the low nanomolar range. In some embodiments, the affinity between the reporter agent/competing binder and the capture agent is in the picomolar range, optionally in the low picomolar range. In some embodiments, the affinity between the reporter agent/competing binder and the capture agent is in the femtomolar range, optionally in the low femtomolar range.

The competing binder may be the same or different from the analyte. In one example, where the capture agent comprises CBD fused to SARS-CoV-2 Receptor Binding Domain (RBD), the competing binder comprises Angiotensin Converting Enzyme-2 (ACE2) protein while the analyte comprises neutralizing antibody that blocks the complex formation of RBD-CBD and ACE2.

In various embodiments, determining a presence or absence of said analyte captured on the cellulose substrate by said capture agent comprises detecting a signal effected by a reporter agent on the one or more test zones, wherein said reporter agent: (i) comprises an analyte binder and said reporter agent is contacted with the sample to allow said reporter agent to bind to said analyte, if present, in said sample; or (ii) comprises a competing binder having affinity for said capture agent and said reporter agent is contacted with said capture agent to allow said reporter agent to bind to said capture agent. For example, said reporter agent may bind to said capture agent (e.g., in large quantities) when said analyte (e.g., antibody) is not present in said sample or when said analyte is only present in small quantities in said sample. In various embodiments, therefore, the method comprises contacting a reporter agent with the sample and/or the capture agent. The contacting step may be carried out prior to or after the step of applying the sample to the one or more test zones. The contacting step may also be carried out simultaneously with or separately from the step of incubating the sample with the capture agent. For example, the reporter agent, the capture agent and the sample may be incubated together. For example, the reporter agent may be incubated with the capture agent or the sample separately from the incubation of the sample with the capture agent before dispensing both incubated contents to the one or more test zones. For example, the reporter agent may be dispensed on the cellulose substrate following the immobilization of the capture agents on the test zones and/or following the application of the sample on the test zones.

In various embodiments, the reporter agent is detectable. In some embodiments, the reporter agent is detectable via a detectable analyte binder, competing binder or binding protein comprised therein. For example, an analyte binder, competing binder or binding protein may be directly detectable, or it may be indirectly detectable e.g. it is capable of e.g. interacting with a reactant to produce a detectable signal or forming a complex that generates a detectable signal. In some embodiments, the reporter agent is detectable via a label.

A label may be any molecule or moiety that facilitates detection of the reporter agent (and therefore detection of any analyte competing binder bound to the reporter agent). A label may be capable of effecting a detectable signal, including signals that are visible and/or signals that are detectable using suitable sensor or instrumentation. Suitable labels depend on the particular assay format and/or detection system and may include those that are well known to those skilled in the art. Many labels are commercially available and may be used in embodiments of the method. Common types of labels include optical labels (e.g. fluorescent, luminescent and/or light-scattering labels, colorimetric labels, coloured molecules such as colloidal nanoparticles, molecules that generate colours during a reaction), radioactive labels, magnetic and/or electrical labels, enzymes, ligands with specific bonds, microscopic vesicles detectable by acoustic resonance, and the like. Examples of a label include, but are not limited to, a dye, a fluorescent dye, a fluorophore, a luminophore, a chromophore, a chromatogen, a chemiluminescent compound, a catalyst, an enzyme (e.g. a peroxidase), an enzymatic reagent/substrate, a biotin, a radioisotope, a mass label, a charge label, a spin label, a receptor, a ligand, a nanoparticle (e.g., gold, silver, carbon nanotubes), a colloidal metallic particle, a colloidal non-metallic particle or other moiety known in the art that can be measured by an analytical method and combinations thereof. A label can be directly detectable by the eye and/or by suitable sensor/instrumentation, or it can be detectable as a result of subsequent processes. For example, a label in the form of a dye may be directly detectable by the eye and/or by suitable sensor/instrumentation, while a label in the form of an enzyme such as horseradish peroxidase (HRP) become detectable when it produces a colour change (that may correlate with the analyte level) upon reaction with a substrate such as 3,3′, 5,5″-tetramethylbenzidine (TMB).

In various embodiments therefore, detecting a signal effected by the reporter agent may be direct (e.g. by the eye and/or by suitable sensor/instrumentation) or indirect (e.g. through enzymatic reaction of a substrate by the reporter agent).

In one embodiment, the label comprises a fluorescent dye. In one embodiment, the fluorescent dye comprises an Alexa Fluor (produced by Invitrogen Corporation) based dye molecule. In one example, the fluorescent dye comprises Alexa Fluor 594. In one embodiment, the label comprises an enzyme that is capable of producing a colorimetric signal/readout. In one embodiment, the enzyme comprises a peroxidase. In one embodiment, the peroxidase comprises HRP. Advantageously, the use of HRP is convenient as it can produce a colorimetric signal/readout that is visible/discernible by the naked/unaided eye. The optical density of the colorimetric signal/readout can further be quantified/measured spectrophotometrically. Furthermore, HRP is capable of amplifying a signal and increasing the detectability of a reporter agent. HRP is also stable, and relatively resistant to heat and organic solvent.

A label may be coupled/attached directly to an analyte binder, competing binder or binding protein in a reporter agent, or it may be coupled/attached indirectly to an analyte binder, competing binder or binding protein in a reporter agent through one or more bridges or secondary reporter agents. For example, a label may be coupled/conjugated directly to an analyte binder, competing binder or binding protein in a reporter agent. In one embodiment, a label in the form of a fluorescent dye is coupled/conjugated to a competing binder to form the reporter agent. For example, a label may be coupled to an analyte binder, competing binder or binding protein through a biotin-streptavidin bridge or biotin-avidin bridge, or through a secondary antibody. In one embodiment, an analyte binder or binding protein is tagged with biotin and allowed to interact with HRP-conjugated streptavidin (SA-HRP) to produce a reporter agent in the form of a HRP-labelled analyte binder, competing binder or binding protein coupled through a biotin-streptavidin bridge. Advantageously, an indirect coupling/attachment of a label to an analyte binder or binding protein through e.g. the use of one or more bridges or secondary reporter agents may enhance/amplify a signal and thereby improve the detection sensitivity and/or the limit of detection of the method. For example, biotin and streptavidin has a dissociation constant (K_{d}) in the femtomolar range and the strong affinity between the two molecules allow more analytes to be coupled successfully to a label for detection. In one embodiment therefore, the reporter agent comprises a label coupled to an analyte binder, competing binder or binding protein through a biotin-streptavidin or biotin-avidin bridge. Advantageously, the biotin-streptavidin and biotin-avidin systems not only enhance/amplify a signal, but the systems are also versatile and compatible with a wide variety of analyte binders or binding proteins and labels.

In various embodiments, the reporter agent comprises an analyte binder, competing binder or binding protein and a label coupled thereto. In various embodiments, the reporter agent may further comprise one or more bridges. In various embodiments, the reporter agent comprises an analyte binder, competing binder or binding protein coupled to a label through one or more bridges. In various embodiments, the analyte binder, competing binder or binding protein is tagged with a biotin or biotinylated. The analyte binder, competing binder or binding protein may be further coupled or fused to one or more partners or moieties, e.g. a maltose-binding protein (MBP) to increase biotin accessibility. In various embodiments, the label is coupled/conjugated to streptavidin or avidin. In various embodiments, the reporter agent comprises an analyte binder, competing binder or binding protein coupled to a label through a biotin-streptavidin or biotin-avidin bridge. It may be appreciated that a label may already be present in the reporter agent e.g. at the incubating/contacting step or the reporter agent may be devoid of a label and is coupled to a label e.g. during or after the incubating/contacting step. In some embodiments therefore, the method comprises providing a reporter agent comprising a label. In some embodiments, the method comprises a step of adding/coupling a label (e.g. a streptavidin-conjugated label or a streptavidin-conjugated label) to an analyte binder, competing binder or binding protein (e.g. a biotinylated analyte binder, competing binder or binding protein), and optionally incubating/contacting the two, to obtain a reporter agent comprising/coupled to a label or a labelled reporter agent. The label adding/coupling step may be performed before, during or after the sample incubating step. The label adding/coupling step may be performed prior to the step of incubating the reporter agent with the capture agent. In some embodiments, a label is incubated with the sample and the analyte binder, competing binder or binding protein to obtain a reporter agent comprising/coupled a label or a labelled reporter agent (e.g. through biotin-streptavidin or biotin-avidin mediated coupling of the label to the analyte binder, competing binder or binding protein).

In various embodiments, the method further comprises adding a reagent capable of producing a detectable signal upon interaction/reaction with the reporter agent after the applying step. Suitable reagents depend on the particular reporter agent (e.g. the particular analyte binder, competing binder or binding protein and/or the particular label). For example, where the reporter agent comprises a HRP label, the reagent may be a HRP substrate that gives a detectable (e.g. coloured) end product upon reaction with HRP. In some embodiments therefore, where the label comprises HRP, the method comprises adding a reagent selected from the group consisting of o-Phenylene Diamine (OPD), 2,2′-azino-bis(3-ethylbenzthiazoline-6-sulphonate (ABTS), 3-amino-9-ethylcarbazole (AEC), 4-Chloro-1-Naphtol (4-CN), 3,3″,5,5″-tetramethylbenzidine (TMB), 3,3′-DiAminoBenzimidine (DAB), 10-Acetyl-3,7-Dihydroxyphenoxazine (ADHP), Resazurin, Luminol, Uptilight tyramide and combinations thereof.

In various embodiments, the method comprises a step of adding a label (e.g. SA-HRP) prior or after the applying step and a subsequent step of adding a reagent (e.g. TMB) for expressing the signal.

In various embodiments, the signal/readout effected or produced by the reporter agent or label include an optical signal/readout (e.g. colorimetric signal/readout, fluorescence, iluminescence such as chemiluminescence, electrochemiluminescence and photoluminescence etc.), radioactivity, infrared radiation, resonance energy transfer (FRET), emission signal/readout, magnetic and/or electrical signal/readout, acoustic signal/readout and any other forms of signal/readout that can be produced by a label or an analyte binder, competing binder or binding protein as described herein. In some embodiments, the signal/readout comprises a colorimetric signal/readout. In some embodiments, the colorimetric signal/readout is visible/discernible/distinguishable to the naked/unaided eye. In some embodiments, the signal/readout comprises a fluorometric signal/readout. In some embodiments, the intensity of the fluorometric/colorimetric signal/readout correlates with the level/amount/concentration of the analyte in the sample. In some embodiments, the intensity of the fluorometric/colorimetric signal/readout positively correlates with the level/amount/concentration of the analyte in the sample i.e., the higher the intensity of the fluorometric/colorimetric signal/readout, the higher the level/amount/concentration of the analyte in the sample. For example, a darker or more intense colour, or a higher fluorescence intensity produced by a reporter agent comprising an analyte binder may indicate that a sample contains a higher level/amount/concentration of the analyte than a lighter/fainter or less intense colour or a lower fluorescence intensity. In some embodiments, the intensity of the of the fluorometric/colorimetric signal/readout negatively correlates with the level/amount/concentration of the analyte in the sample (e.g., in a competitive binding assay) i.e., the higher the intensity of the fluorometric/colorimetric signal/readout, the lower the level/amount/concentration of the analyte in the sample. For example, a darker or more intense colour, or a higher fluorescence intensity produced by a reporter agent comprising a competing binder (e.g., in a competitive binding assay) may indicate that a sample contains a lower level/amount/concentration of the analyte than a lighter/fainter or less intense colour or a lower fluorescence intensity.

In some embodiments, the signal/readout is quantifiable. In some embodiments, detecting a signal/readout effected or produced by the reporter agent or label comprises capturing the signal/readout with a camera and optionally analysing the captured image to e.g. measure/quantify the intensity of the signal/readout. The image analysis may be carried out, for example, by use of ImageJ software or other suitable imaging software programs or algorithms.

In various embodiments, wherein where the reporter agent comprises an analyte binder, the sample is incubated with the reporter agent under conditions that allow the analyte, if present in the sample, to be bound by the reporter agent. In various embodiments, the sample is incubated with the reporter agent for a sufficient time to allow binding of any analyte therein to the reporter agent. The duration of incubation/interaction may depend on the binding affinity of the reporter agent for the analyte as well as the concentration of the analyte. In various embodiments, the sample is incubated with the reporter agent for not less than about 15 seconds, not less than about 30 seconds, not less than about 45 seconds, not less than about 60 seconds, not less than about 75 seconds, not less than about 90 seconds, not less than about 105 seconds, not less than about 120 seconds, not less than about 135 seconds, not less than about 150 seconds, not less than about 165 seconds, not less than about 180 seconds, not less than about 195 seconds, not less than about 210 seconds, not less than about 225 seconds, not less than about 240 seconds, not less than about 255 seconds, not less than about 270 seconds, not less than about 285 seconds or not less than about 300 seconds. In various embodiments, the sample is incubated with the reporter agent for about 15 seconds to about 300 seconds, about 15 seconds to about 180 seconds, from about 30 seconds to about 180 seconds, from about 45 seconds to about 180 seconds, from about 60 seconds to about 180 seconds, from about 15 seconds to about 120 seconds, from about 30 seconds to about 120 seconds, from about 45 seconds to about 120 seconds or from about 60 seconds to about 120 seconds. In one embodiment, the sample is incubated with the reporter agent for more than about 10 seconds. In one embodiment, the sample is incubated with the reporter agent for at least about 60 seconds. Advantageously, in various embodiments, the sample is allowed a sufficient amount of time to interact with the reporter agent for the effective formation of a reporter agent-analyte complex. More analytes may be successfully bound to a reporter agent at the early stage and this may improve the detectability of the analytes in the later detection stage of the method. Consequently, sensitivity of the assay/method may be increased and false negative results may be reduced. The limit of detection may also be improved.

In various embodiments, wherein where the reporter agent comprises a competing binder, the reporter agent is incubated with the capture agent under conditions that allow the reporter agent to be bound by the capture agent. The duration of interaction/incubation may depend on the dissociation constant (Kd) between the reporter agent and the capture agent, and the Kd between the analyte and the capture agent if the sample is incubated together with the reporter agent and the capture agent. In various embodiments, the capture agent is incubated with the reporter agent for not less than about 15 seconds, not less than about 30 seconds, not less than about 45 seconds, not less than about 60 seconds, not less than about 75 seconds, not less than about 90 seconds, not less than about 105 seconds, not less than about 120 seconds, not less than about 135 seconds, not less than about 150 seconds, not less than about 165 seconds or not less than about 180 seconds, not less than about 195 seconds, not less than about 210 seconds, not less than about 225 seconds, not less than about 240 seconds, not less than about 255 seconds, not less than about 270 seconds, not less than about 285 seconds or not less than about 300 seconds. In various embodiments, the capture agent is incubated with the reporter agent for about 15 seconds to about 300 seconds, about 15 seconds to about 180 seconds, about 30 seconds to about 180 seconds, about 45 seconds to about 180 seconds, about 60 seconds to about 180 seconds, about 15 seconds to about 120 seconds, about 30 seconds to about 120 seconds, about 45 seconds to about 120 seconds or about 60 seconds to about 120 seconds. In one embodiment, the capture agent is incubated with the reporter agent for more than about 10 seconds. In one embodiment, the capture agent is incubated with the reporter agent for at least about 60 seconds. Advantageously, in various embodiments, the reporter agent is allowed a sufficient amount of time to interact with the capture agent for the effective formation of a reporter agent-capture agent complex. More reporter agents can be successfully bound to a capture agent to be captured onto the cellulose substrate. False positive results may be reduced.

In various embodiments, the incubating step is not carried out/performed on a strip/paper strip/test strip i.e. the incubating step is carried out/performed off site a strip/paper strip/test strip. In various embodiments, the incubating step is not carried out/performed on a cellulose substrate or cellulose paper i.e. the incubating step is carried out/performed off site a cellulose substrate or cellulose paper. In various embodiments, the incubating step is carried out in solution. In various embodiments, the incubating step is carried out in a solvent. In various embodiments, the incubating step comprises adding the sample, the reporter agent and/or the capture agent to a solvent. In various embodiments, the solvent offers solubility to the analyte, the reporter agent and/or the capture agent. In various embodiments, the solvent is capable of solubilizing the analyte, the reporter agent and/or the capture agent. Examples of suitable solvents include, but are not limited to, phosphate-buffered saline (PBS); lysis buffer (e.g. buffer containing surfactant or detergent); buffer containing stabilizer e.g. glycerol, sucrose, trehalose; biological fluids (e.g. blood, plasma, saliva, etc.); water; liquid component of food; liquid component of environmental samples; and the like etc. In various embodiments, the solvent is selected from the group consisting of: buffer solution, biological fluid, water, liquid component of food, liquid component of environmental samples and combinations thereof.

After incubation, the incubated sample may be applied to/contacted with a cellulose substrate to allow any reporter agent-analyte complex to be captured onto the cellulose substrate by the capture agent or to allow any reporter agent-capture agent complex to be captured onto the cellulose substrate.

The cellulose substrate may be any substrate that incorporates cellulose. Examples include, but are not limited to, cellulose paper, nitrocellulose paper, and any other cellulose-based/cellulose fiber-based papers and the like. A cellulose substrate further includes any functionalised cellulose substrates, such as cellulose substrates which surface are functionalised with e.g. nanoparticles.

The cellulose substrate may have one or more, or a plurality of test zones thereon. In various embodiments therefore, applying the sample to a cellulose substrate comprises applying the sample to a test zone on the substrate. In various embodiments, the analyte is to be captured in a test zone on the substrate. In various embodiments, the capture agent is immobilised in a test zone on the cellulose substrate. In various embodiments, the test zone is substantially circular in shape. In one embodiment, the test zone comprises a round microzone. It will be appreciated that the shape of the test zones is not particularly limited to a circular/round shape and the test zones may also be of other shapes e.g., square, rectangle, oval, ellipsoid, triangle, parallelogram or the like or a combinations thereof. In such embodiments, the shape of the openings on the lid may be configured to correspond to the shape of the test zones. The shapes of the plurality of test zones on the substrate may be the same or different. In one embodiment, the shapes of the plurality of test zones on the substrate are the same. When a plurality of substrates is used, the shape and/or size of the test zones on one substrate may be the same or different from the shape and/or size of the test zones on another substrate. When a plurality of substrates is used, the shape and/or size of the test zones on/within each substrate may be the same or different. In one embodiment, the shape of the test zones and/or size on every substrate are the same.

It will be appreciated that the sizes/area/diameters of the test zones are not particularly limited and may be varied according to the requirements of the assay. When a plurality of substrates is used (e.g., in a vertical flow assay), the size/area/diameter/width of the test zones on one substrate may be the same or different from the size/area/diameter/width of the test zones on another substrate. When a plurality of substrates is used, the size/area/diameter/width of the test zones on each substrate may be the same or different. In one embodiment, the size/area/diameter/width of the test zones on every substrate are the same. In one embodiment, the size/area/diameter/width of the test zones on every substrate are different. In one embodiment, wherein where the one or more layers of cellulose substrate comprises a plurality of layers of cellulose substrate, the diameter of the test zones of a cellulose substrate selected from the plurality of layers of cellulose substrate is different from the diameter of the test zones of another cellulose substrate selected from the plurality of layers of cellulose substrate

In various embodiments, the test zones of the cellulose substrate are relatively more permeable to a liquid sample than areas of the cellulose substrate that are outside the test zones. For example, the areas of the cellulose substrate that are outside the test zones are coated with layer that is substantially impermeable to a liquid sample. Advantageously, the relative impermeability of areas outside the test zones may allow for the sample to concentrate in the test zones, subsequently allowing results of the assays to be focused on the test zones. Cross-contamination between test zones may also be minimised/prevented. The relative impermeability of areas outside the test zones may also substantially prevent adsorption of molecules in these areas.

Accordingly, the washing buffer may be used to easily wash unbounded molecules from these areas. This may advantageously improve the specificity of the assay by reducing the likelihood of non-specific binding in the areas outside the test zones, which may interfere with the accuracy of the results read-out or detection.

In various embodiments, the layer that is substantially impermeable to a liquid sample comprises a hydrophobic layer. Accordingly, in various embodiments, the cellulose substrate comprises a waxed cellulose substrate (e.g., waxed cellulose paper). In various embodiments, the test zones of the cellulose substrate are not waxed. The waxed cellulose substrate may be obtained by applying printing techniques such as wax printing and/or screen printing. For example, a printer using hydrophobic polymer solutions and/or wax ink may be used to create hydrophobic patterns on cellulose paper (e.g., filter paper) to create defined hydrophobic barriers around the unprinted zones which can eventually serve as hydrophilic test zones. Examples of suitable wax ink include, but are not limited to, Fuji Xerox Colorqube Solid ink and polystyrene ink (alkyl ketyl dimer in p-xylene).

In some embodiments, no more than one cellulose substrate is used e.g., in a lateral flow assay.

In some embodiments, a plurality of stacked cellulose substrates are used e.g., in a vertical flow assay. In various embodiments, at least about 2 cellulose substrates, at least about 3 cellulose substrates, at least about 4 cellulose substrates, or at least about 5 cellulose substrate are stacked together e.g., to form at least about 2 layers, at least about 3 layers, at least about 4 layers or at least about 5 layers of the cellulose substrates. In some embodiments, a cellulose substrate is folded one or more times to form multiple layers for a vertical flow assay. In various embodiments, the cellulose substrate is folded to form at least about 2 layers, at least about 3 layers, at least about 4 layers or at least about 5 layers of the cellulose substrate. In various embodiments, one or layers of an absorbent material may be disposed underneath the plurality of stacked cellulose substrates or the multiple layers of a cellulose substrate such that the sample/fluid will pass through the cellulose substrate before reaching the absorbent material. Advantageously, the absorbent material may serve to remove excess unbound molecules and washing buffer via capillary force and gravity. The absorbent material may also provide a wicking/pulling force to enhance the flow of the sample and subsequently the completion of the assay. In one embodiment, the absorbent material comprises Whatman GB005 Whatman blotting paper.

Nonetheless, it will be appreciated that other types of absorbent material or blotting paper may be used as long as they may adequately remove excess unbound molecules and washing buffer during the assays. For example, other porous material (not limited to cellulose materials) may also be used e.g., ReliaFlow 440 from Ahlstrom-Munksjö made of a mix of cotton and glass fibres. In various embodiments, at least about 1 layer of absorbent material, at least about 2 layers of absorbent material, at least about 3 layers of absorbent material, or at least about 4 layers of absorbent material are disposed underneath the plurality of stacked cellulose substrates or the multiple layers of a cellulose substrate.

In various embodiments, the number of test zones in a layer of cellulose substrate are no more than the number of test zones in the layer immediately below it. In various embodiments, the number of test zones in each layer of cellulose substrate are the same.

The test zones in each layer may also be substantially aligned to the corresponding test zones of the other layers. In various embodiments, where the test zones in one layer are of same or different sizes from the test zones of another layer, the center of each test zone in one layer and the center of each respective corresponding test zone in the other layer may be substantially aligned (e.g., a line that is passing through both centers is substantially perpendicular to the surface of the cellulose substrate on which the test zones reside and/or the bottom plate). For example, if the test zones in all layers are circular in shape, the test zones in each layer may be aligned such that the test zones on are substantially concentric to corresponding test zones in another layer. In various embodiments, the centers of the corresponding test zones in all of the different layers are substantially aligned to one another (e.g., substantially concentric to one another).

The flow rate of the sample (and/or any other reagents added to the test zones) passing through the cellulose substrates may be optimized for signal generation. Without being bound by theory, it is believed that the flow rate of a sample (and/or any other reagents) depends on two factors: (i) the binding affinity of protein of interest/analyte in the sample with its capture agent and (ii) the viscosity of the sample. This can be adjusted by (a) changing the test zone sizes across the cellulose substrate layers to alter the capillary effect and gravity flow and/or (b) changing the viscosity of the sample (and/or any other reagents added to the test zones).

In various embodiments, when there is a plurality of layers of cellulose substrate present, the test zones in each layer of substrate may be varied to adjust the flow rate of the sample through the test zones. For instance, the size of the test zones in each layer of cellulose substrate may be different. In such a situation, due to the difference in the sizes of the overlapping hydrophilic areas of the test zones between the layers (e.g., between 1^{st} and 2^{nd} layers and between 2^{nd} and 3^{rd} layers etc), the flow rate of the sample through the cellulose substrates may be adjusted. For example, decreasing diameters of the test zones down the layers (i.e., from the top most layer to the bottom most layer) reduces a flow rate of the sample through the layers.

It will be appreciated that instead of only relying on the difference in size of the test zones between layers, other manners of varying the test zones in each layer may also be useful to vary the sample flow rate across the layers. For example, varying the alignment between the corresponding test zones of each layer of the cellulose substrate (e.g. such that the centers of the respective test zones between layers may be offset from each other or misaligned) may in turn result in the overlapping hydrophilic areas of the test zones between the layers (e.g. between 1^{st} and 2^{nd} layers and between 2^{nd} and 3^{rd} layers etc) being varied, thereby allowing the flow rate of the sample through the cellulose substrates to be adjusted. In various embodiments, the capture agent is capable of recognising and/or binding to the analyte. In various embodiments, the capture agent has binding affinity for the analyte. In various embodiments, the capture agent is specific to the analyte. In various embodiments, the capture agent comprises an analyte binder or binding protein that is capable of binding to the analyte. In various embodiments, the affinity between the capture agent and the analyte is not more than about 10⁻⁶ M, not more than about 10⁻⁷ M, not more than about 10⁻⁸ M, not more than about 10⁻⁹ M, not more than about 10-10 M, not more than about 10⁻¹¹ M, not more than about 10⁻¹² M, not more than about 10⁻¹³ M, not more than about 10⁻¹⁴ M, not more than about 10⁻¹⁵ M or less than about 10⁻¹⁵ M. In some embodiments, the affinity between the capture agent and the analyte is in the micromolar range, optionally in the low micromolar range. In some embodiments, the affinity between the capture agent and the analyte is in the nanomolar range, optionally in the low nanomolar range. In some embodiments, the affinity between the capture agent and the analyte is in the picomolar range, optionally in the low picomolar range. In some embodiments, the affinity between the capture agent and the analyte is in the femtomolar range, optionally in the low femtomolar range.

In various embodiments, the capture agent is capable of recognising and/or binding to the reporter agent e.g., a reporter agent comprising a competing binder having affinity for the capture agent. In various embodiments, the capture agent has binding affinity for the reporter agent. In various embodiments, the capture agent is specific to the reporter agent. In various embodiments, the affinity between the capture agent and the reporter agent is not more than about 10⁻⁶ M, not more than about 10⁻⁷ M, not more than about 10⁻⁸ M, not more than about 10⁻⁹ M, not more than about 10-10 M, not more than about 10⁻¹¹ M, not more than about 10⁻¹² M, not more than about 10⁻¹³ M, not more than about 10⁻¹⁴ M, not more than about 10⁻¹⁵ M or less than about 10⁻¹⁵ M. In some embodiments, the affinity between the capture agent and the reporter agent is in the micromolar range, optionally in the low micromolar range. In some embodiments, the affinity between the capture agent and the reporter agent is in the nanomolar range, optionally in the low nanomolar range. In some embodiments, the affinity between the capture agent and the reporter agent is in the picomolar range, optionally in the low picomolar range. In some embodiments, the affinity between the capture agent and the reporter agent is in the femtomolar range, optionally in the low femtomolar range.

In various embodiments, the capture agent comprises a cellulose binding domain (CBD). CBD is a protein domain that is present in many carbohydrate active enzymes. CBDs have been classified into at least 13 families named I-XIII according to their amino acid sequence similarities, with most of the reported CBDs belonging to families I, II, and III. The CBD that may be incorporated in the capture agent is not particularly limited to CBDs of certain families. In various embodiments, the CBD may be selected from the group consisting of: a family I CBD, a family II CBD, a family III CBD, a family IV CBD, a family V CBD, a family VI CBD, a family VII CBD, a family VIII CBD, a family VIIII CBD, a family X CBD, a family XI CBD, a family XII CBD, a family XIII CBD and combinations thereof. In one embodiment, the CBD comprises a family III CBD. In one embodiment, the CBD comprises a CBD from a CBD3a family. The CBD may be a naturally occurring CBD, or it may be a modified/synthesised CBD having affinity for cellulose.

In various embodiments, the CBD comprises from about 20 to about 200 amino acid residues, from about 30 to about 40 amino acid residues, from about 80 to about 110 amino acid residues, from about 90 to about 100 amino acid residues, from about 120 to about 180 amino acid residues or from about 130 to about 180 amino acid residues. In some embodiments, the CBD comprises from about 150 to about 170 amino acid residues. In one embodiment, the CBD comprises about 160 amino acid residues.

In some embodiments, the CBD comprises/consists of the SEQ ID NO: 1 (PVSGNLKVEFYNSNPSDTTNSINPQFKVTNTGSSAIDLSKLTLRYYYTVDGQK DQTFWCDHAAIIGSNGSYNGITSNVKGTFVKMSSSTNNADTYLEISFTGGTLE PGAHVQIQGRFAKNDWSNYTQSNDYSFKSASQFVEWDQVTAYLNGVLVWG KEP) or portions, optionally linear portions, thereof or a sequence sharing at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% or at least about 99% sequence identity thereto. In some embodiments, the CBD is encoded by SEQ ID NO: 2 (CCGGTATCAGGCAATTTGAAGGTTGAATTCTA CAACAGCAATCCTTCAGATACTACTAACTCAATCAATCCTCAGTTCAAGGTT ACTAATACCGGAAGCAGTGCAATTGATTTGTCCAAACTCACATTGAGATATT ATTATACAGTAGACGGACAGAAAGATCAGACCTTCTGGTGTGACCATGCTG CAATAATCGGCAGTAACGGCAGCTACAACGGAATTACTTCAAATGTAAAAG GAACATTTGTAAAAATGAGTTCCTCAACAAATAACGCAGACACCTACCTTG AAATAAGCTTTACAGGCGGAACTCTTGAACCGGGTGCACATGTTCAGATAC AAGGTAGATTTGCAAAGAATGACTGGAGTAACTATACACAGTCAAATGACT ACTCATTCAAGTCTGCTTCACAGTTTGTTGAATGGGATCAGGTAACAGCAT ACTTGAACGGTGTTC TTGTATGGGGTAAAGAACCC) or portions, optionally linear portions, thereof or a sequence sharing at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% or at least about 99% sequence identity thereto. A portion, optionally a linear portion, of SEQ ID NO:1 or 2 may comprise one or more stretches of the amino acid residues in SEQ ID NO: 1 or one or more stretches of the bases or nucleotides in SEQ ID NO: 2 that is capable of encoding a protein/peptide product that can bind to cellulose with high affinity. The stretch of residues or bases/nucleotides may comprise at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 105, at least about 110, at least about 115, at least about 120, at least about 125, at least about 130, at least about 135, at least about 140, at least about 145 or at least about 150 successive residues or bases/nucleotides in SEQ ID NO:1 or 2.

In various embodiments, the CBD has a binding affinity for cellulose that is not more than about 10⁻⁶ M, not more than about 10⁻⁷ M, not more than about 10⁻⁸ M, not more than about 10⁻⁹ M, not more than about 10⁻¹⁰ M, not more than about 10⁻¹¹ M, not more than about 10⁻¹²M, not more than about 10⁻¹³M, not more than about 10⁻¹⁴ M, not more than about 10⁻¹⁵ M or less than about 10⁻¹⁵ M. In some embodiments, the CBD has a binding affinity for cellulose that is about 1 μM or less. In some embodiments, the CBD has a binding affinity for cellulose that is from about 0.5 μM to about 1 μM. In some embodiments, the CBD comprises a family I CBD having a binding affinity for cellulose that is from about 0.5 μM to about 1 μM.

In some embodiments, the CBD is located at an end or at an extremity of the capture agent. In some embodiments, the CBD is attached or coupled to an end or an extremity of an analyte binder or a binding protein in the capture agent. In some embodiments, the CBD is attached or coupled to a terminus of the analyte binder or binding protein for the analyte. In some embodiments, the CBD is attached or coupled to a C-terminus of the analyte binder or binding protein for the analyte. In various embodiments, the positioning of the CBD within the capture agent advantageously allows the capture agent to be immobilised onto the cellulose substrate in an optimal orientation, e.g. with the binding faces facing away from the cellulose substrate and/or towards the applied sample, such that the binding faces are available for contacting and binding to the analyte. Consequently, more capture agents can bind the analytes successfully, and detectability of the analytes may be increased. The sensitivity of the assay/method may be increased and false negative results can be reduced. The limit of detection may also be improved. In some embodiments therefore, a plurality of capture agents is immobilised on the cellulose substrate in a substantially uniform orientation.

The capture agent may be incubated with the sample prior to the applying step and/or immobilised on the cellulose substrate prior to the applying step.

In some embodiments, the capture agent is incubated with the sample prior to the applying step. The capture agent may be incubated with the sample separately from the reporter agent (e.g. before and after the sample is incubated with the reporter agent), and/or it may be incubated with the sample together with the reporter agent. In some embodiments, wherein where the reporter agent comprises an analyte binder, the sample is first incubated/contacted with the capture agent to form a capture agent-analyte complex and then subsequently incubated/contacted with the reporter agent to form a full sandwich complex in the form of a reporter agent-analyte-capture agent complex. The sample containing the full sandwich complex may then be applied/dispensed onto the cellulose substrate to allow the full sandwich complex to be immobilised on the cellulose substrate (via affinity between the CBD of the capture agent and the cellulose substrate) for detection. In some embodiments, wherein where the reporter agent comprises an analyte binder, the sample is first incubated/contacted with the reporter agent to form a reporter agent-analyte complex and then subsequently incubated/contacted with the capture agent to form a full sandwich complex in the form of a reporter agent-analyte-capture agent complex. The sample containing the full sandwich complex may then be applied/dispensed onto the cellulose substrate to allow the full sandwich complex to be immobilised on the cellulose substrate for detection. In some embodiments, wherein where the reporter agent comprises an analyte binder, the sample is incubated/contacted with both the reporter agent and the capture agent, and optionally a label, at the same time (i.e. together) to form a full sandwich complex in the form of a reporter agent-analyte-capture agent complex. The sample containing the full sandwich complex may then be applied/dispensed onto the cellulose substrate to allow the full sandwich complex to be immobilised on the cellulose substrate for detection. In some embodiments therefore, wherein the capture agent is incubated with the sample prior to the applying step, the analyte that is bound to the reporter agent is part of a reporter agent-analyte-capture agent complex (i.e. a full sandwich complex) prior to the applying step.

In some embodiments therefore, wherein where said reporter agent comprises an analyte binder, the method comprises incubating the reporter agent and the capture agent with said sample prior to the applying step and said analyte when present, is bound to the reporter agent to form part of a reporter agent-analyte-capture agent complex prior to the applying step.

In some embodiments, wherein where the reporter agent comprises a competing binder, the sample is first incubated/contacted with the capture agent to form a capture agent-analyte complex and then subsequently incubated/contacted with the reporter agent to form a capture agent-reporter agent complex. The sample containing both the capture agent-analyte complex and the capture agent-reporter agent complex may then be applied/dispensed onto the cellulose substrate to allow the complexes to be immobilised on the cellulose substrate (via affinity between the CBD of the capture agent and the cellulose substrate) for detection. In some embodiments, where the reporter agent comprises a competing binder, the reporter agent is first incubated/contacted with the capture to form a capture agent-reporter agent complex and then subsequently incubated/contacted with the sample to form a capture agent-analyte complex. The sample containing both the capture agent-analyte complex and the capture agent-reporter agent complex may then be applied/dispensed onto the cellulose substrate to allow the complexes to be immobilised on the cellulose substrate for detection. In some embodiments, where the reporter agent comprises a competing binder, the sample is incubated/contacted with both the reporter agent and the capture agent, and optionally a label, at the same time (i.e., together) to form both the capture agent-analyte complex and the capture agent-reporter agent complex. The sample containing both the capture agent-analyte complex and the capture agent-reporter agent complex may then be applied/dispensed onto the cellulose substrate to allow the complexes to be immobilised on the cellulose substrate for detection.

In some embodiments therefore, wherein where said reporter agent comprises a competing binder, the method comprises incubating said capture agent with said reporter agent and/or said sample prior to the applying step to form a reporter agent-capture agent complex and/or an analyte-capture agent complex prior to the applying step.

In various embodiments, the sample is incubated with the capture agent under conditions that allow the analyte, if present in the sample, to be bound by the capture agent. In various embodiments, the capture agent is incubated with the sample for not less than about 15 seconds, not less than about 30 seconds, not less than about 45 seconds, not less than about 60 seconds, not less than about 75 seconds, not less than about 90 seconds, not less than about 105 seconds, not less than about 120 seconds, not less than about 135 seconds, not less than about 150 seconds, not less than about 165 seconds, not less than about 180 seconds, not less than about 195 seconds, not less than about 210 seconds, not less than about 225 seconds, not less than about 240 seconds, not less than about 255 seconds, not less than about 270 seconds, not less than about 285 seconds or not less than about 300 seconds. In various embodiments, the capture agent is incubated with the sample for about 15 seconds to about 300 seconds, about 15 seconds to about 180 seconds, about 30 seconds to about 180 seconds, about 45 seconds to about 180 seconds, about 60 seconds to about 180 seconds, about 15 seconds to about 120 seconds, about 30 seconds to about 120 seconds, about 45 seconds to about 120 seconds or about 60 seconds to about 120 seconds. In one embodiment, the capture agent is incubated with the sample for more than about 10 seconds. In one embodiment, the capture agent is incubated with the sample for at least about 60 seconds. Advantageously, in various embodiments, the sample is allowed a sufficient amount of time to interact with the capture agent for the effective formation of a capture agent-analyte complex or if the reporter agent is present, a reporter agent-analyte-capture agent complex. More analytes can be successfully bound to a capture agent to be captured onto the cellulose substrate. This may improve the detectability of the analytes. Consequently, sensitivity of the assay/method may be increased and false negative results may be reduced. The limit of detection may also be improved.

In some embodiments, the capture agent is immobilised on the cellulose substrate prior to the applying step. In some embodiments, the method comprises adding/dispensing capture agents onto the cellulose substrate to allow the capture agents to be immobilised on the cellulose substrate before the applying step e.g. before, after or at the same time with the incubating step. The capture agent may be pre-immobilised on the cellulose substrate and/or it may be added/dispensed onto the cellulose substrate prior to the applying step. In some embodiments, the method comprises providing a cellulose substrate with the capture agents immobilised/disposed thereon e.g. before the incubating step or applying step. Advantageously, embodiments of the method allows for the application to the substrate a full-sandwich complex in the form of a reporter agent-analyte-capture agent complex, a half-sandwich complex in the form of a reporter agent-analyte complex, or in the case of a competitive binding assay, a capture agent-reporter agent complex and/or a capture agent-analyte complex

In various embodiments, wherein where said reporter agent comprises an analyte binder, the method comprises incubating the reporter agent with said sample and immobilizing said capture agent on said cellulose substrate prior to the applying step and said analyte when present, is bound to the reporter agent to form a reporter agent-analyte complex prior to the applying step.

In various embodiments, wherein where said reporter agent comprises a competing binder, the method comprises immobilizing said capture agent on said cellulose substrate prior to the applying step and dispensing the reporter agent on the cellulose substrate (e.g., on one or more test zones of the cellulose substrate) after the applying step. In various embodiment, where said reporter agent comprises a competing binder, the method comprises incubating said reporter agent with said sample and immobilizing said capture agent on said cellulose substrate prior to the applying step and dispensing the reporter agent incubated with said sample on said cellulose substrate after the applying step.

Embodiments of the method employing a capture agent having CBD have several advantages. CBD has very high affinity towards cellulose substrate in which the interaction or binding between the CBD and the cellulose can happen in less than a second. This increases the efficiency of the method. For example, after a full sandwich complex (i.e. a reporter agent-analyte-capture agent complex) is applied to the cellulose substrate, less waiting time may be required before detection of sandwich complex captured by the cellulose substrate can be made. For example, where a capture agent is applied to the cellulose substrate, less waiting time may also be required to immobilise the capture agent onto the cellulose substrate before the next step, e.g. the application of the sample to the cellulose substrate, may be carried out. Further, the immobilisation of a conventional capture agent devoid of a CBD onto a cellulose substrate typically occurs spontaneously through hydrophobic and electrostatic interactions and such interactions offer only moderate strength. Thus, some capture agents may be lost. There is also no control over the orientation of the capture agents on the cellulose substrate. As a result, the capture agents are immobilised on the substrate in random orientations with the binding faces facing non-uniform directions. Collectively, the loss of capture agents and random orientations of the capture agents on the substrate may reduce the amount of capture agents available for binding to the analytes or reporter agents and assay sensitivity and/or accuracy may be reduced. By contrast, embodiments of the capture agent comprising a CBD has much higher affinity for the cellulose substrate, thus allowing more capture agents to be immobilised on the cellulose substrate. Thus, in various embodiments, less capture agents are lost, if any. The specific binding of the CBD to the cellulose may also advantageously orient the capture agents such that their binding faces are directed towards the applied analytes or reporter agents. Collectively, this can increase the availability of the capture agents for capturing analytes or reporter agents. The sensitivity of a non-competitive assay/method may be increased and false negative results may be reduced. The limit of detection may also be improved. In a competitive assay/method, the accuracy of the method may also be increased and false positive results may be reduced. Further, with less capture agents being lost because of inadequate binding strength, the use of the capture agents may also be better optimised. Less capture agents may be required to capture a given amount of analyte or reporter agent, reducing wastage of resources and cost of the method.

Embodiments of the method employing a capture agent having CBD is also a relatively safe and easy alternative to immobilize proteins on a cellulose substrate as compared to using functionalized cellulose paper for immobilizing proteins. The synthesis of functionalized cellulose paper requires periodate, which is a dangerous chemical, for oxidation of cellulose to make carboxylic groups.

In various embodiments, the amount of capture agents used is not more than about 1000-fold molar excess, not more than about 900-fold molar excess, not more than about 800-fold molar excess, not more than about 700-fold molar excess, not more than about 600-fold molar excess, not more than about 500-fold molar excess, not more than about 400-fold molar excess, not more than about 300-fold molar excess, not more than about 200-fold molar excess, not more than about 100-fold molar excess, not more than about 90-fold molar excess, not more than about 80-fold molar excess, not more than about 70-fold molar excess, not more than about 60-fold molar excess, not more than about 50-fold molar excess, not more than about 40-fold molar excess, not more than about 30-fold molar excess, not more than about 20-fold molar excess, not more than about 10-fold molar excess, not more than about 9-fold molar excess, not more than about 8-fold molar excess, not more than about 7-fold molar excess, not more than about 6-fold molar excess or not more than about 5-fold molar excess of the analytes or of the typical amount range of analytes that is contained in a typical sample of a similar/identical type as the sample. In some embodiments, the capture agent is not more than 1000-fold molar excess, optionally not more than 100-fold molar excess, further optionally not more than 60-fold molar excess of said analyte. In various embodiments, the ratio of the moles of the capture agents to the moles of the analyte (or the typical molar range of analyte that is contained in a typical sample of a similar/identical type as the sample) is not more than about 1000, not more than about 900, not more than about 800, not more than about 700, not more than about 600, not more than about 500, not more than about 400, not more than about 300, not more than about 200, not more than about 100, not more than about 90, not more than about 80, not more than about 70, not more than about 60, not more than about 50, not more than about 40, not more than about 30, not more than about 20, not more than about 10, not more than about 9, not more than about 8, not more than about 7, not more than about 6 or not more than about 5. Surprisingly, embodiments of the method are demonstrated to have better sensitivity than comparative examples using a greater excess of the capture agents. The surprising results may be attributed to the longer incubation time between the sample and the reporter agents and/or the capture agents in embodiments of the method. As compared to the use of an abundant excess of capture agents, a longer incubation time may be more effective in maximising/increasing the amount of full sandwich complex captured successfully on the cellulose substrate, and therefore more effective n increasing assay sensitivity. Resource wastage and cost are also reduced in embodiments of the method.

In various embodiments, the capture agent and/or the reporter agent may comprise an analyte binder or a binding protein for the analyte. The analyte binder or the binding protein may be capable of binding to the analyte. In various embodiments, the analyte binder or the binding protein is capable of binding to the analyte with substantially high specificity and/or high affinity. In various embodiments, the affinity between the analyte binder or the binding protein is not more than about 10⁻⁶ M, not more than about 10⁻⁷ M, not more than about 10⁻⁸ M, not more than about 10⁻⁹ M, not more than about 10⁻¹⁰ M, not more than about 10⁻¹¹ M, not more than about 10⁻¹² M, not more than about 10⁻¹³ M, not more than about 10⁻¹⁴ M, not more than about 10⁻¹⁵ M or less than about 10⁻¹⁵ M. In some embodiments, the affinity between the analyte binder or the binding protein is in the micromolar range, optionally in the low micromolar range. In some embodiments, the affinity between the analyte binder or the binding protein is in the nanomolar range, optionally in the low nanomolar range. In some embodiments, the affinity between the analyte binder or the binding protein is in the picomolar range, optionally in the low picomolar range. In some embodiments, the affinity between the analyte binder or the binding protein is in the femtomolar range, optionally in the low femtomolar range. In some embodiments, the reporter agent and/or capture agent comprises an analyte binder or a binding protein having picomolar affinity for the analyte.

The analyte binder, competing binder or the binding protein may be an antibody or a non-antibody. Examples of analyte binders, competing binders or binding proteins include, but are not limited to antibodies (polyclonal and/or monoclonal), antigen binding proteins, peptides, aptamers, haptens, receptors, engineered proteins, engineered protein scaffolds such as AdNectin, Affibody, Anticalin, Knottin, DARPin, Kunitz, affitins, fibronectins and other organic and/or polymeric scaffolds, fragments thereof (e.g. antigen- or analyte-binding fragments thereof) and the like. As used herein, the term “antibody” refers to an immunoglobulin or fragment thereof, and encompasses any polypeptide comprising an antigen-binding fragment or an antigen-binding domain. The term includes but is not limited to polyclonal, monoclonal, monospecific, polyspecific (such as bi-specific), humanized, human, single-chain, chimeric, synthetic, recombinant, hybrid, mutated, grafted, and in vitro generated antibodies. The term “antibody” may include antibody fragments such as Fab, F(ab′)2, Fv, scFv, Fd, dAb, and other antibody fragments that retain antigen-binding function. An antibody is not necessarily from any particular source, nor is it produced by any particular method.

In some embodiments, the analyte binder, competing binder or the binding protein comprises a non-antibody analyte binder, non-antibody competing binder or a non-antibody binding protein. In some embodiments, the non-antibody analyte binder, the non-antibody competing binder or the non-antibody binding protein comprises a protein scaffold. In some embodiments, the protein scaffold comprises a protein originating, based on or derived from Sulfolobus genera. In some embodiments, the protein scaffold comprises an affitin. Affitins are highly stable engineered affinity proteins. As background, affitins are originally derived from the 7 kDa DNA-binding polypeptides, comprising Sac7d and Sso7d, from Sulfolobus genera. Positively charged amino acids are removed from affitin creating reduced charge affitins (rcSac7d and rcSso7d) that can bind to other types of molecules other than DNA. By randomizing the amino acids on the binding surface of the polypeptides e.g. rcSac7d or rcSso7d, and subjecting the resulting protein library to rounds of yeast surface display, the affinity can be directed towards various targets including peptides, proteins, viruses, and bacteria. Advantageously, affitins are small (typically about 7 kDa), durable, highly soluble, highly selective, cost effective, resistant to extreme alkaline pH and chemically and thermally stable. In various embodiments therefore, the analyte binder, the competing binder or binding protein, optionally the non-antibody analyte binder, the non-antibody competing binder or non-antibody binding protein has one or more of the following properties: small size (for example, smaller than the a typical antibody having a size of 130-150 kDa), durable (e.g. able to withstand many cycles of purification), highly soluble, highly selective, cost effective, resistant to extreme alkaline pH, chemically stable and thermally stable. In various embodiments, the affitin is based on or derived from a protein of the Sul7d family. In various embodiments, the affitin is Sac7d-based, or Sac7d-derived, or Sso7d-based or Sso7d-derived. In one embodiment, the affitin is Sso7d-based or Sso7d-derived. Embodiments of the affitin may be wholly or partially isolated from a naturally occurring Sso7d or Sac7d directly, or more typically, it may be wholly synthesized using the amino acid sequence of a Sso7d or Sac7d as a prototype/reference (and then subjected to further mutation or modification and selection by yeast surface display to the generate an affitin having affinity to the analyte of interest). In some embodiments, the affitin comprises rcSso7d protein.

In various examples, the affitin is specific to a coronavirus protein, optionally SARS-CoV-2 protein, further optionally a SARS-CoV-2 nucleocapsid protein. In various embodiments therefore, the analyte comprises a coronavirus protein, optionally SARS-CoV-2 protein. In one embodiment, the analyte comprises SARS-CoV-2 nucleocapsid protein or fragments thereof. In various embodiments, the method is a method of detecting coronavirus, optionally severe acute respiratory syndrome coronavirus 2 (SARS-CoV-2) in a sample or in a subject from which the sample is obtained from. In various embodiments, the method is a method of detecting an infection, optionally a virus infection, further optionally a coronavirus infection, further optionally COVID-19 infection in a sample or in a subject from which the sample is obtained from.

In various embodiments, the method comprises a diagnostic method. The method may be implemented/performed as an immunoassay method, an enzyme-linked immunosorbent assay (ELISA) method, a lateral flow assay/method, a vertical flow assay/method, a rapid diagnostic test method, a point-of-care diagnostic method or the like. In various embodiments, the method is a lateral flow assay method or a vertical flow assay method. In one embodiment, the method comprises a point-of-care lateral flow method. In one embodiment, the method comprises a point-of-care vertical flow method. In one example, the method is performed using a form of vertical flow assay whereby the sample and the reagent solutions were flowed in a top-to-bottom direction. In various embodiments, the method is implemented/performed in a point-of-care device. In various embodiments, the method is implemented/performed in a lateral flow device or a vertical flow device. In various embodiments, there is provided a device for implementing

Embodiments of the methods may apply the principles of antibody/antigen interactions to perform the assays, although they are not limited/restricted as such. For example, embodiments of the methods may also rely on non-antibody ligand based interaction. Such flexibility allow for a wide array of assay possibilities.

In various embodiments, the method is a vertical flow assay and the sample applying step comprises allowing the sample to flow through a plurality of cellulose substrates layers e.g., to an absorbent material.

Advantageously, embodiments of the method provide rapid results. In various embodiments, the results (e.g. the presence, absence, amount, relative amount of the analyte) may be obtained within about 4 hours, within about 3.5 hours, within about 3 hours, within about 2.5 hours, within about 2 hours, within about 1.5 hour, within about 1 hour, within about 55 minutes, within about 50 minutes, within about 45 minutes, within about 40 minutes, within about 35 minutes, within about 30 minutes, within about 25 minutes, within about 20 minutes, within about 15 minutes, within about 10 minutes, within about 9 minutes, within about 8 minutes, within about 7 minutes, within about 6 minutes or within about 5 minutes from the start of the incubating step. In one embodiment, the presence or absence of the analyte in the sample is determined within 15 minutes from the start of the incubating step.

The method may further comprise applying/contacting a control sample to the cellulose substrate to obtain an indication on whether the method is correctly performed. The control sample may comprise a positive control sample, a negative control sample, or both. As may be appreciated, a detection or an immobilization of a positive control sample may indicate that the method is correctly performed, for example, a binding of the reporter and capture agents to the analyte is successful e.g. during the incubation step and that the result obtained is not a false negative due to unsuccessful binding e.g. during the incubation step. No detectable immobilization of a negative control sample may indicate that the method is correctly performed, for example, there is no non-specific interaction and that the result obtained is not a false positive. In some embodiments, the method comprises processing the control sample prior to applying/contacting it with the cellulose substrate. As may be appreciated, methods of processing the sample, as herein disclosed before, may apply to the control sample.

In various embodiments, the method may be implemented as a system. In various embodiments, there is provided a system for detecting an analyte in a sample, the system comprising: an incubation mixture comprising said sample and a reporter agent configured to bind to said analyte; and/or a cellulose substrate configured to capture the analyte that is bound to said reporter agent, via a capture agent comprising a cellulose binding domain (CBD), wherein said capture agent is: (i) present in the incubation mixture; and/or (ii) pre-immobilised on said cellulose substrate, and wherein the presence or absence of said analyte captured on said cellulose is determinable by detection via the reporter agent. In various embodiments, there is provided a system for detecting an analyte in a sample, the system comprising: a cellulose substrate configured to capture the analyte and reporter agent, via a capture agent comprising a cellulose binding domain (CBD), wherein said capture agent is: (i) present in an incubation mixture comprising said sample and a reporter agent configured to bind to the capture agent; and/or (ii) pre-immobilised on said cellulose substrate, and wherein the presence or absence of said analyte captured on said cellulose is determinable by detection via the reporter agent. In various embodiments, the system further comprises the incubation mixture comprising said sample and the reporter agent configured to bind to the capture agent comprising a cellulose binding domain (CBD). In various embodiments, the system may further comprise one or more of the following: an image capturing device/unit/module for capturing an image of the cellulose substrate; a processing device/unit/module for processing data obtained from the image and a storage medium for storing instructions to analyse the data from the image. In one embodiment, all of these modules/units can be part of a single mobile/portable device e.g., a mobile phone, and the instructions are part of an application installed in the mobile/portable device.

In various embodiments, said incubation mixture comprises said sample that has been incubated with said reporter agent for not less than about 15 seconds, not less than about 30 seconds, not less than about 45 seconds, not less than about 60 seconds, not less than about 75 seconds, not less than about 90 seconds, not less than about 105 seconds, not less than about 120 seconds, not less than about 135 seconds, not less than about 150 seconds, not less than about 165 seconds or not less than about 180 seconds. In various embodiments, said incubation mixture comprises said sample that has been incubated with said reporter agent for about 15 seconds to about 180 seconds, from about 30 seconds to about 180 seconds, from about 45 seconds to about 180 seconds, from about 60 seconds to about 180 seconds, from about 15 seconds to about 120 seconds, from about 30 seconds to about 120 seconds, from about 45 seconds to about 120 seconds or from about 60 seconds to about 120 seconds. In one embodiment, said incubation mixture comprises said sample that has been incubated with said reporter agent for more than about 10 seconds. In one embodiment, said incubation mixture comprises said sample that has been incubated with said reporter agent for at least about 60 seconds. In some embodiments, said incubation mixture comprises said sample that has been incubated with said reporter agent for not less than 15 seconds, optionally not less than 30 seconds, further optionally not less than 45 seconds, further optionally not less than 60 seconds.

In various embodiments, the capture agent is incubated with the sample in the incubation mixture. In various embodiments, the sample is incubated with the capture agent for not less than about 15 seconds, not less than about 30 seconds, not less than about 45 seconds, not less than about 60 seconds, not less than about 75 seconds, not less than about 90 seconds, not less than about 105 seconds, not less than about 120 seconds, not less than about 135 seconds, not less than about 150 seconds, not less than about 165 seconds or not less than about 180 seconds. In various embodiments, the sample is incubated with the capture agent for about 15 seconds to about 180 seconds, from about 30 seconds to about 180 seconds, from about 45 seconds to about 180 seconds, from about 60 seconds to about 180 seconds, from about 15 seconds to about 120 seconds, from about 30 seconds to about 120 seconds, from about 45 seconds to about 120 seconds or from about 60 seconds to about 120 seconds. In one embodiment, the sample is incubated with the capture agent for more than about 10 seconds. In one embodiment, the sample is incubated with the capture agent for at least about 60 seconds. In some embodiments, where said capture agent is incubated with said sample in said incubation mixture, the sample has been incubated with said capture agent for not less than 15 seconds, optionally not less than 30 seconds, further optionally not less than 45 seconds, further optionally not less than 60 seconds.

In various embodiments, the amount of capture agent present in the incubation mixture and/or pre-immobilised on the cellulose substrate is not more than about 1000-fold molar excess, not more than about 900-fold molar excess, not more than about 800-fold molar excess, not more than about 700-fold molar excess, not more than about 600-fold molar excess, not more than about 500-fold molar excess, not more than about 400-fold molar excess, not more than about 300-fold molar excess, not more than about 200-fold molar excess, not more than about 100-fold molar excess, not more than about 90-fold molar excess, not more than about 80-fold molar excess, not more than about 70-fold molar excess, not more than about 60-fold molar excess, not more than about 50-fold molar excess, not more than about 40-fold molar excess, not more than about 30-fold molar excess, not more than about 20-fold molar excess, not more than about 10-fold molar excess, not more than about 9-fold molar excess, not more than about 8-fold molar excess, not more than about 7-fold molar excess, not more than about 6-fold molar excess or not more than about 5-fold molar excess of the analytes or of the typical amount range of analytes that is contained in a typical sample of a similar/identical type as the sample. In some embodiments, the amount of capture agent present in the incubation mixture and/or pre-immobilised on the cellulose substrate is not more than about 1000-fold molar excess, optionally not more than 100-fold molar excess, further optionally not more than 60-fold molar excess of the analyte. In various embodiments, the ratio of the moles of the capture agents to the moles of the analyte (or the typical molar range of analyte that is contained in a typical sample of a similar/identical type as the sample) in the incubation mixture is not more than about 1000, not more than about 900, not more than about 800, not more than about 700, not more than about 600, not more than about 500, not more than about 400, not more than about 300, not more than about 200, not more than about 100, not more than about 90, not more than about 80, not more than about 70, not more than about 60, not more than about 50, not more than about 40, not more than about 30, not more than about 20, not more than about 10, not more than about 9, not more than about 8, not more than about 7, not more than about 6 or not more than about 5.

Embodiments of the method comprising an incubation step and/or use of a capture agent comprising a CBD advantageously lead to improvement in analyte detection sensitivity. In a comparative example, the pre-incubation of analytes, reporter agents and/or CBD-tagged capture reagents comprising CBD to form complexes before subsequent application of the complexes to cellulose paper was found to result in significantly greater colorimetric intensity production as compared to the direct application of analytes and reporter agents (without incubation) to a cellulose paper with CBD-tagged capture reagents immobilised thereon. In the direct application example, the analytes, reporter agent and CBD-tagged capture reagents have only a very short interaction time (˜1-3 sec) with each other. Hence, the leads to inefficient binding of the reporter agents to the analytes and also the inefficient capturing of the analytes to the cellulose paper by the capture agents. Consequently, the sensitivity of the assay is relatively low, as evidenced by the low colorimetric intensity production. In contrast, without being bound by theory, it is believed that incubation promotes the effective binding between the analytes and reporter agents and/or capture agents by allowing for sufficient interaction time between the analytes and reporter agents and/or capture agents. This increases the amount of full sandwich complexes successfully formed and captured onto the cellulose substrate, thereby increasing the analyte detection sensitivity. In another comparative example, a capture agent without CBD (specifically rcSso7d without CBD) was found to bind to cellulose paper 2000 times less than a capture agent comprising a CBD (specifically CBD-tagged rcSso7d) at 1 to 10 seconds. This finding indicates that a capture agent comprising a CBD is able to bind to a cellulose substrate much more efficiently than a capture agent devoid of a CBD. Analyte detection sensitivity is therefore increased with the use of a capture agent comprising a CBD, since a greater amount of capture agents can be successfully bound to the cellulose substrate. Collectively, an incubation step and/or use of a capture agent comprising a CBD lead to significant improvement in analyte detection sensitivity.

In various embodiments, there is provided a kit for detecting an analyte in a sample, the kit comprising: an incubation solution/mixture/solvent (e.g. for incubating and/or binding said sample and a reporter agent that is configured to bind to said analyte in a non-competitive binding assay, or for incubating and/or binding said sample and a reporter agent that is configured to bind to said capture agent in a competitive binding assay); and/or a cellulose substrate (e.g. configured to capture the analyte that is bound to said reporter agent in a non-competitive assay or configured to capture both the analyte and reporter agent in a competitive binding assay). In some embodiments, the kit further comprises a capture agent. In some embodiments, the cellulose substrate comprises a capture agent, optionally a capture agent comprising a CBD, immobilised thereon. In some embodiments, the kit further comprises a capture agent provided separately from the cellulose substrate. In some embodiments, the kit further comprises a reagent capable of producing a detectable signal upon reaction with the reporter agent. In various embodiments, the reporter agent, the capture agent and/or the analyte is soluble in the incubation solution/mixture/solvent.

In various embodiments, there is provided a method of identifying a coronavirus infection, optionally a COVID-19 infection in a subject, the method comprising: incubating a sample obtained from the subject with a reporter agent having binding affinity to a coronavirus protein, optionally SARS-CoV-2 protein, further optionally SARS-CoV-2 nucleocapsid protein or fragments thereof to allow said reporter agent to bind to said protein or fragments, if present, in said sample; applying the incubated sample to a cellulose substrate to allow said protein that is bound to said reporter agent to be captured onto said cellulose substrate by a capture agent comprising a cellulose binding domain (CBD), wherein said capture agent is: (i) incubated with said sample prior to the applying step; and/or (ii) immobilised on said cellulose substrate prior to the applying step; and detecting a signal effected by the reporter agent to determine the presence or absence of said protein captured on said cellulose substrate by said capture agent and/or the presence or absence of said infection in the subject. Embodiments of the method, system and kit may also be used for detecting neutralizing antibodies against a coronavirus infection, optionally a COVID-19 infection in a subject, for example, in a competitive binding assay format using SARS-CoV-2 Receptor Binding Domain (RBD) fused with CBD as the capture agent and labelled ACE2 as the reporter agent. As used herein, the term “identifying” as used herein in relation to a medical condition (such as an infection) is to be interpreted broadly to encompass determining a presence, an absence and/or a severity of the medical condition. The term “subject” as used herein includes patients and non-patients. The term “patient” refers to individuals suffering or are likely to suffer from a medical condition such as an infection, while “non-patients” refer to individuals not suffering and are likely to not suffer from the medical condition. “Non-patients” include healthy individuals, non-diseased individuals and/or an individual free from the medical condition. The term “subject” includes humans and animals. Animals include murine and the like. “Murine” refers to any mammal from the family Muridae, such as mouse, rat, and the like. The term “SARS-CoV-2” includes a virus having the sequence set forth in SEQ ID NO: 3, as well as variants/mutants thereof that share at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.91%, at least about 99.92%, at least about 99.93%, at least about 99.94%, at least about 99.95%, at least about 99.96%, at least about 99.97%, at least about 99.98% or at least about 99.99% sequence identity with the sequence over its entire length. Examples of SARS-CoV-2 variants/mutants include, but are not limited to, Alpha (B.1.1.7), Beta (B.1.351, B.1.351.2, B.1.351.3), Delta (B.1.617.2, AY.1, AY.2, AY.3), and Gamma (P.1, P.1.1, P.1.2) variants.

In addition to detection of analytes or diagnostics, the interaction between CBD and cellulose substrate as described herein may also be applied to a wide array of biomedical and life science applications. For example, one application is protein separation. Interaction between CBD and cellulose substrate occurs instantaneously. Thus, in some examples, a protein of interest may be fused to CBD and then separated/isolated rapidly using cellulose substrate for further downstream analysis.

### Methods for Identifying an Infection

Embodiments of the method of detecting an analyte may be used to identify an infection in a subject. For example, embodiments of the method may be used to detect a coronavirus infection, such as a SARS-CoV-2 infection, in a subject

In various embodiments therefore, there is provided a method of detecting SARS-CoV-2 infection in a subject, the method comprising: incubating a sample from the subject with a reporter agent to allow said reporter agent to bind to a SARS-CoV-2 protein (or fragments thereof), if present, in said sample; applying the incubated sample to a cellulose substrate to allow the SARS-CoV-2 protein that is bound to said reporter agent to be captured onto said cellulose substrate by a capture agent comprising a cellulose binding domain (CBD), wherein said capture agent is: (i) incubated with said sample prior to the applying step; and/or (ii) immobilised on said cellulose substrate prior to the applying step; and detecting a signal effected by the reporter agent to determine a presence or absence of said SARS-CoV-2 protein captured on said cellulose substrate by said capture agent. Examples of a SARS-CoV-2 protein include the SARS-CoV-2 membrane (M) protein, the SARS-CoV-2 envelope (E) protein, the SARS-CoV-2 spike (S) protein and the SARS-CoV-2 nucleocapsid (N) protein. In one example, said SARS-CoV-2 protein comprises the SARS-CoV-2 nucleocapsid (N) protein and said reporter agent is configured to bind to the SARS-CoV-2 nucleocapsid (N) protein.

In some embodiments, the capture agent is immobilised on said cellulose substrate prior to the applying step and the analyte (e.g., the SARS-CoV-2 protein), if present in the sample, is part of a reporter agent-analyte complex (i.e., a half-sandwich complex). In some examples, the reporter agent is incubated with the sample for no less than about 300 seconds, no less than about 270 seconds, no less than about 240 seconds, no less than about 210 seconds, no less than about 180 seconds, no less than about 150 seconds, no less than about 120 seconds, no less than about 90 seconds, no less than about 60 seconds or no less than about 30 seconds.

In some embodiments, said capture agent is incubated with said sample prior to the applying step. For example, at the time of incubating the sample with the reporter agent, the capture agent may also be added to the incubation mixture to allow for the formation of a full sandwich complex in the form of a reporter agent-analyte-capture agent complex prior to the applying step. In some examples, the incubation time is no less than about 300 seconds, no less than about 270 seconds, no less than about 240 seconds, no less than about 210 seconds, no less than about 180 seconds, no less than about 150 seconds, no less than about 120 seconds, no less than about 90 seconds, no less than about 60 seconds or no less than about 30 seconds. Advantageously, this test format (i.e., the full complex cellulose pulldown (CP-F) format) is shown to produce the strongest signal intensity and the highest ‘signal-to-noise’ ratio as compared to the short incubation time format, the limited incubation time format and the half complex cellulose pulldown (CP-H) format in an example.

In various embodiments, the method further comprises performing a control reaction. In various embodiments, performing a control reaction comprises immobilising the SARS-CoV-2 protein (e.g., the SARS-CoV-2 N protein) on a designated control spot on the cellulose substrate. In one example, N protein fused to CBD at its C terminus (NP-CBD) is immobilised on a designated control spot on the cellulose substrate.

In various embodiments, the method further comprises a step of washing the cellulose substrate (or test zones and/or control zones on the cellulose substrate), for example, with a washing reagent/buffer, after the sample applying step to minimize non-specific signals on the cellulose surface. The washing step may be performed before the detecting step.

In various embodiments, both the reporter agent and the capture agent have affinity for the SARS-CoV-2 protein. In various embodiments, the reporter agent and the capture agent are configured to recognise and/or bind to different epitopes of the SARS-CoV-2 protein. In various embodiments, the reporter agent and the capture agent are configured to bind to different epitopes of the SARS-CoV-2 protein.

In various embodiments, the reporter agent and/or the capture agent do not comprise antibodies. Therefore, embodiments of the method do not employ antibodies. In various embodiments, the reporter agent and/or the capture agent may be produced using a bacterial system, which is more cost-effective as compared to the mammalian cell line system required for antibody production. In various embodiments, the reporter agent and/or the capture agent comprises an affitin. In various embodiments, the affitin comprises rcSso7d protein.

In various embodiments, the reporter agent, the capture agent and/or the affitin comprises SEQ ID NO: 4 (MATVKFTYQGEEKQVDISKIKIVRRGGQWISFWYDEGGGAYGAGYVSEKDA PKELLQMLEKQ), SEQ ID NO: 5 (MATVKFTYQGEEKQVDISKIKNVGRWGQIIDFDYDEGGGAIGIGAVSEKDAPK ELLQMLEKQ) or a sequence sharing at least about at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8% or at least about 99.9% sequence similarity/identity thereto. In some embodiments, the reporter agent, the capture agent and/or the affitin comprises an amino acid sequence that differs from the sequence set forth in SEQ ID NO: 4 or SEQ ID NO: 5 by about one amino acid residue, about two amino acid residues, about three amino acid residues, about four amino acid residues, about five amino acid residues, about six amino acid residues, about seven amino acid residues, about eight amino acid residues, about nine amino acid residues, about ten amino acid residues or more. In some embodiments, the reporter agent, the capture agent and/or the affitin comprises an amino acid sequence that differs from the sequence set forth in SEQ ID NO: 4 or SEQ ID NO: 5 by no more than about one amino acid residue, no more than about two amino acid residues, no more than about three amino acid residues, no more than about four amino acid residues, no more than about five amino acid residues, no more than about six amino acid residues, no more than about seven amino acid residues, no more than about eight amino acid residues, no more than about nine amino acid residues or no more than about ten amino acid residues. The differing amino acid residue(s) may be at any position from position 1 to position 62 of SEQ ID NO: 4 or SEQ ID NO: 5. In various embodiments, the differing amino acid residue(s) is at position 22, position 24, position 26, position 29, position 31, position 33, position 41, position 43 and/or position 45 of SEQ ID NO: 4 or SEQ ID NO: 5. In various embodiments, the sequence of the reporter agent differs from the sequence of the capture agent at position 22, position 24, position 26, position 29, position 31, position 33, position 41, position 43 and/or position 45 within a stretch/window of 62 amino acid residues, which may be similar/identical to SEQ ID NO: 4 or SEQ ID NO: 5. In various embodiments, the reporter agent comprises SEQ ID NO: 4. In various embodiments, the capture agent comprises SEQ ID NO: 5.

In various embodiments, the reporter agent comprises a spacer protein. An example of a spacer protein is maltose binding protein (MBP). In various embodiments, the reporter agent comprises a biotin (BA). In various embodiments, the reporter agent comprises a rcSso7d protein coupled/fused to BA and MBP. Tagging of BA-MBP may improve protein stability and allows for biotin-mediated labelling or immobilization. The rcSso7d protein coupled/fused to BA and MBP may be allowed to interact with HRP-conjugated streptavidin (SA-HRP) to produce a reporter agent in the form of a HRP-labelled analyte binder.

In various embodiments, the reporter agent has high binding affinity for a SARS-CoV-2 protein. In one example, the dissociation constant (K_{D}) of the reporter agent for SARS-CoV-2 N protein is no more than about 10 nM, no more than about 8 nM, no more than about 5 nM, no more than about 4 nM. no more than about 3.5 nM or no more than about 3.2 nM. In one example, the dissociation constant (K_{D}) of the reporter agent for SARS-CoV-2 N protein is about 3.2 nM.

In various embodiments, the capture agent comprises a rcSso7d protein coupled/fused to a cellulose binding domain (CBD). In various embodiments, the capture agent has high binding affinity for a SARS-CoV-2 protein. In one example, the dissociation constant (K_{D}) of the capture agent for SARS-CoV-2 N protein is no more than about 10 nM, no more than about 8 nM, no more than about 5 nM, no more than about 4.5 nM or no more than about 4.2 nM. In one example, the dissociation constant (K_{D}) of the reporter agent for SARS-CoV-2 N protein is about 4.2 nM.

The sample may be selected from the group consisting of a saliva sample, a sputum sample, a nasal fluid sample, a pharyngeal fluid sample, a nasopharyngeal fluid sample and an oropharyngeal fluid sample. In various embodiments, the sample comprises a saliva sample. Advantageously, collection of a saliva sample from a subject is easy and less-invasive than nasal and nasopharyngeal swabs. Accordingly, embodiments of the method may be suitably employed for frequent testing of an infection such as a SARS-CoV-2 infection. In various embodiments, the method further comprises a step of reducing the viscosity of the sample e.g., the saliva sample. In one embodiment, the sample e.g., the saliva sample is filtered (e.g., with a 5 μm filter) to reduce viscosity.

In various embodiments, the method further comprises treating the sample with a reagent, such as a detergent, that is capable of breaking a viral membrane. Breaking a viral membrane may release the protein of interest, e.g., the N protein, and allow for its detection.

Embodiments of the method are compatible for use with cellulose paper as the cellulose substrate. Advantageously, cellulose papers are cost-effective and readily available, unlike nitrocellulose substrates which currently face high demand. The cellulose substrate may comprise one or more test zones/spots and/or one or more control zones/spots. In one example, a cellulose test strip is folded in a ‘zig-zag’ manner to form a layered cellulose paper acting as the cellulose substrate and secured with additional cellulose paper(s) acting as absorbent pad(s) underneath the layered cellulose paper.

In various embodiments, detecting a signal effected by the reporter agent comprises imaging the cellulose substrate, for example, using a camera. In one example, a phone camera is used. After an image is obtained, the image may be analysed to determine the signal intensity (e.g., intensity of colour) and thereby determine the presence of the infection and/or an amount of viral load in the subject. In some embodiments, signal intensity is measured by an absorbance reader or a spectrophotometer (e.g., a spectrophotometer configured to measure absorbance at a certain wavelength).

In various embodiments, the assay is in the form of a vertical flow assay. A vertical flow assay may have a short fluid/liquid flow path as compared to a lateral flow assay, thus allowing for rapid and controllable flow speed.

Advantageously, embodiments of the method are suitable for point-of-care applications. In various embodiments, the method has a short turnaround time of no more than about 20 minutes, no more than about 18 minutes, no more than about 15 minutes, no more than about 12 minutes, no more than about 10 minutes, no more than about 8 minutes or no more than about 5 minutes. In various embodiments, the method has a short turnaround time of from about 5 minutes to about 10 minutes.

In various embodiments, the method comprises a one-pot reaction prior to the applying step.

Embodiments of the method have good sensitivity and/or specificity. In some examples, the method has a limit of detection (LoD) of no more than about nM, no more than about 4 nM, no more than about 3 nM, no more than about 2.5 nM, no more than about 2 nM, no more than about 1.5 nM or no more than about 1 nM. In one example, the LoD is about 2.5 nM. In one example, the LoD is about 1 nM. In one example, the LoD is no more than about 4.0×10³ TCID50/mL, no more than about 1.0×10³ TCID50/mL, no more than about 5.0×10⁴ TCID50/mL or no more than about 4.0×10⁴ TCID50/mL. In one example, the LoD is about 4.0×10⁴ TCID50/mL. Embodiments of the method may identify highly infectious subjects or subjects with a high viral load. In one example, the method has 100% specificity against the 18 pathogens as shown in FIG. 28D. Thus, in various embodiments, the reporter agent and/or the capture agent does not cross-react with or does not bind to one or more of the 18 pathogens or its components.

In various embodiments, there is provided a system or kit for detecting SARS-CoV-2 infection in a subject. The system or kit may comprise a reporter agent and a capture agent as described herein and/or an incubation mixture comprising a reporter agent and a capture agent as described herein. The system or kit may further comprise a cellulose substrate as described herein. In various embodiments, the system may further comprise one or more of the following: an image capturing device/unit/module for capturing an image of the cellulose substrate; a processing device/unit/module for processing data obtained from the image and a storage medium for storing instructions to analyse the data from the image. In one embodiment, all of these modules/units can be part of a single mobile/portable device e.g., a mobile phone, and the instructions are part of an application installed in the mobile/portable device.

### Methods for Detecting an Immunity Status Against an Infection

Embodiments of the method of detecting an analyte may also be used to detect an antibody against an infection in a subject. For example, embodiments of the method may be used to detect an antibody against a coronavirus infection, such as a SARS-CoV-2 infection, in a subject. In one example, embodiments of the method are also able to detect antibodies against several variants of SARS-CoV-2.

In various embodiments, there is provided a method of detecting an antibody against SARS-CoV-2 in a subject, the method comprising: applying a sample from the subject to a cellulose substrate to allow said antibody, if present in said sample, to be captured onto said cellulose substrate by a capture agent comprising a cellulose binding domain (CBD), wherein said capture agent is: (i) incubated with said sample prior to the applying step; and/or (ii) immobilised on said cellulose substrate prior to the applying step; contacting the capture agent with a reporter agent to allow said reporter agent to bind to said capture agent, wherein the reporter agent comprises a competing binder having affinity for said capture agent; and detecting a signal effected by the reporter agent to determine a presence or absence of said antibody captured on said cellulose substrate by said capture agent. The antibody may be a neutralizing antibody, for example, an antibody that binds to a receptor binding domain (RBD) of SARS-CoV-2 S protein to disrupt ACE2 receptor—RBD complex formation. The antibody may include, but are not limited to, IgA, IgM, IgG and combinations thereof. Advantageously, embodiments of the method may be used to evaluate herd immunity and/or monitor vaccine efficacy against an infection in a population. Embodiments of the method may also be used to evaluate an immunity status and/or a vaccine efficacy in individual subjects.

In some embodiments, contacting the capture agent with a reporter agent comprises incubating the capture agent with the reporter agent. The sample may be further added into the incubation mixture prior to the applying step to allow for a one-pot reaction among the capture agent, the reporter agent, and the antibody if present in said sample. Advantageously, this allows for efficient formation of RBD-CBD/antibody or RBD-CBD/ACE2 complexes before application to the cellulose substrate, thereby increasing an accuracy of the method. In various embodiments, the incubation time is no less than about 420 seconds, no less than about 390 seconds, no less than about 360 seconds, no less than about 330 seconds, no less than about 300 seconds, no less than about 270 seconds, no less than about 240 seconds, no less than about 210 seconds, no less than about 180 seconds, no less than about 150 seconds, no less than about 120 seconds, no less than about 90 seconds, no less than about 60 seconds or no less than about 30 seconds. In various embodiments, the incubation time is no less than about 7 minutes, no less than about 5 minutes, no less than about 3 minutes or no less than about 1 minute.

In various embodiments, the capture agent comprises CBD coupled/fused to RBD. In various embodiments, the reporter agent comprises ACE2 receptor. The ACE2 receptor may be further tagged with or coupled to biotin (BA). Tagging of BA may allow for biotin-mediated labelling. For example, the ACE2-BA may be allowed to interact with HRP-conjugated streptavidin (SA-HRP) to produce a reporter agent in the form of a HRP-labelled competing binder.

In various embodiments, the reporter agent and the capture agent have high affinity towards each other. In one example, the associated K_{D} is no more than about 20 nM, no more than about 18 nM, no more than about 15 nM or no more than about 13 nM. In one example, the K_{D} is about 12.7 nM.

In various embodiments, the ratio of the concentration of the capture agent to the concentration of the reporter agent is about 1. In one example, the concentration of the capture agent is about 10 nM and the concentration of the reporter agent is about 10 nM.

Embodiments of the method are compatible for use with cellulose paper as the cellulose substrate. Advantageously, cellulose papers are cost-effective and readily available, unlike nitrocellulose substrates which currently face high demand. The cellulose substrate may comprise one or more test zones/spots and/or one or more control zones/spots. In one example, a plurality of cellulose papers are stacked together with the top layer of cellulose paper having a test zone of a smaller diameter and the layer(s) underneath having test zone(s) of a larger diameter. In one example, a folded Kim Wipes paper is disposed underneath the plurality of cellulose papers to act as a absorbent pad.

In various embodiments, the method further comprises performing a control reaction. In various embodiments, performing a control reaction comprises immobilising the capture agent or RBD-CBD at high concentrations on a designated control spot on the cellulose substrate.

In various embodiments, the method further comprises a step of washing the cellulose substrate (or test zones and/or control zones on the cellulose substrate), for example, with a washing reagent/buffer, after the sample applying step and/or after the contacting step to minimize non-specific signals on the cellulose surface. The washing step may be performed before the detecting step.

In various embodiments, detecting a signal effected by the reporter agent comprises imaging the cellulose substrate, for example, using a camera. In one example, a phone camera is used. After an image is obtained, the image may be analysed to determine the signal intensity (e.g., intensity of colour) and thereby determine the presence and/or an amount of antibody in the subject, for example, by comparing the signal intensity to a reference signal intensity. The reference signal intensity may be obtained from, for example, a non-infected sample.

In various embodiments, the assay is in the form of a vertical flow assay. A vertical flow assay may have a short fluid/liquid flow path as compared to a lateral flow assay, thus allowing for rapid and controllable flow speed. In various embodiments, the assay is in the form of a competitive binding assay. Embodiments of the method may rely a principle that the presence of antibody in a sample interferes with RBD/ACE2 receptor complex formation and thereby reduce a reporting signal intensity.

Advantageously, embodiments of the method are suitable for point-of care applications. In various embodiments, the method has a short turnaround time of no more than about 24 hours, no more than about 12 hours, no more than about 6 hours, no more than about 3 hours, no more than about 2 hours, no more than about 1 hour, no more than about 30 minutes, no more than about 20 minutes, no more than about 18 minutes, no more than about 15 minutes, no more than about 12 minutes or no more than about 10 minutes. In various embodiments, the method has a short turnaround time of about 10 minutes.

Embodiments of the method have high sensitivity and/or specificity. In various embodiments, the method has a sensitivity and/or specificity of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% at least about 99% or at least about 100%, e.g., as compared to a current lab-based method. In various embodiments, the limit of detection (LoD) of the method for the antibody no more than about 20 nM, no more than 15 nM, no more than about 10 nM or no more than about 5 nM. In one example, the method has a LoD of about 10 nM. In one example, the method has a LoD of about 5 nM.

In one example, the method is highly specific to SARS-CoV-2 NAbs but not to non-Nabs, for example, non-neutralizing human anti-RBD antibodies. In one example, the method shows minimal cross reactivity to antibodies against other viruses, or to SARS-CoV-2 S and N proteins. Thus, in various embodiments, the method does not show cross reaction with one or more of the antibodies shown in FIG. 12F, or SARS-CoV-2 S protein or SARS-CoV-2 N protein.

Embodiments of the method also have high accuracy. In various embodiments, the method has an accuracy of at least about 80%, at least about 85%, at least about 90%, at least about 91%, at least about 92%, at least about 93%, at least about 94%, at least about 95%, at least about 96%, at least about 97%, at least about 98% at least about 99% or at least about 100%, e.g., as compared to a current lab-based method.

The sample may be selected from the group consisting of: comprises a plasma sample, or a serum sample and a whole blood sample. In some embodiments, the sample comprises/is a plasma sample or a serum sample. In some embodiments therefore, a whole blood sample is collected from a subject and processed to extract the plasma or serum. In one example, the method shows improved LoD for a serum sample as compared to a plasma sample. In some embodiments, the sample comprises a whole blood sample e.g., unprocessed whole blood sample. The whole blood sample may be obtained from fingertip or vein. Where the sample comprises a whole blood sample, HRP may not be used in the reporting agent as the presence of peroxidase in red blood cells may interfere with the colorimetric signal generation. In various embodiments therefore, wherein the sample comprises a whole blood sample, the reporter agent may be devoid of HRP. In various embodiments, wherein the sample comprises a whole blood sample, the reporter agent may comprise a fluorescent label. In one example, the fluorescent label comprises Invitrogen Alexa Fluor 594 dye. Other labels that do not interact with components of a whole blood sample may also be used.

When whole blood sample is used, the capture agent may be incubated with the sample first before it is contacted with the reporter agent. This allows any antibody in the sample to interact with the capture agent first for more effective formation of antibody-capture agent complex.

In some embodiments, the sample comprises a convalescent sample or a sample obtained from a subject in a convalescent phase/stage of the infection.

In some embodiments, the subject comprises a subject having the infection (i.e., an infected subject). In some embodiments, the subject comprises a previously infected subject. The subject may have been infected more than about 1 month, more than about 2 months, more than about 3 months, more than about 6 months, more than about 9 months or more than about 12 months ago. In some embodiments, the subject comprises a vaccinated subject. The subject may have been administered a vaccine more than about 1 month, more than about 2 months, more than about 3 months, more than about 6 months, more than about 9 months or more than about 12 months ago. In some embodiments, the subject comprises an elderly subject e.g., a subject who is more than about 60, more than about 65 or more than about 70 years old. In some embodiments, the subject comprises a subject having a chronic medical condition, e.g., diabetes, high blood pressure etc.

Embodiments of the method may be capable of distinguishing between different levels of antibodies. Thus, in various embodiments, the method is a quantitative or semi-quantitative method.

In various embodiments, there is provided a system or kit for detecting an antibody against an infection in a subject. The system or kit may comprise a reporter agent and a capture agent as described herein and/or an incubation mixture comprising a reporter agent and a capture agent as described herein. The system or kit may further comprise a cellulose substrate as described herein. In various embodiments, the system may further comprise one or more of the following: an image capturing device/unit/module for capturing an image of the cellulose substrate; a processing device/unit/module for processing data obtained from the image and a storage medium for storing instructions to analyse the data from the image. In one embodiment, all of these modules/units can be part of a single mobile/portable device e.g., a mobile phone, and the instructions are part of an application installed in the mobile/portable device.

In various embodiments, there is provided a method, a system or a product as described herein.

### BRIEF DESCRIPTION OF FIGURES

FIG. 1. The scheme of a conventional sandwich complex for analyte detection.

FIG. 2. A conventional rapid diagnostic test (RDT) format.

FIG. 3. A scheme of a capture agent and a sandwich complex in accordance with embodiments disclosed herein. (A) a capture agent comprising a cellulose binding domain (CBD) and (B) a sandwich complex comprising such a capture agent in accordance with embodiments disclosed herein.

FIG. 4. A comparison between a conventional RDT format and a cellulose pull down assay format in accordance with an embodiment disclosed herein.

FIG. 5. A scheme of a cellulose pull down full complex method in accordance with an embodiment disclosed herein.

FIG. 6. A scheme of a cellulose pull down half complex method in accordance with an embodiment disclosed herein.

FIG. 7. The colorimetric results of different reagent interaction times on cellulose papers in an example. (A) very short incubation time (SI), (B) limited incubation time (LI), (C) sufficient incubation time for formation of half sandwich complex and pull down using pre-immobilized capture agent comprising CBD (i.e. cellulose pull down half complex—CP-H) and (D) sufficient incubation time for formation of full sandwich complex and pull down using high interaction affinity between CBD and cellulose substrate (i.e. cellulose pull down full complex—CP-F).

FIG. 8. The colorimetric results obtained from different assay formats. (A) an assay in a comparative example (B) a cellulose pull down half complex (CP-H) method in accordance with an embodiment disclosed herein, (C) a cellulose pull down full complex (CP-F) method in accordance with an embodiment disclosed herein and (D) a combination of the CE and CP-H method in accordance with an embodiment disclosed herein.

FIG. 9. Overview of the test construction and image acquiring devices. (A) Exploded scheme of the cellulose testing unit, comprising 2 pieces of acrylic manifold to hold the testing unit together, 3 layers of the folded and printed cellulose test strip whereby hydrophobic ink (black area) was used to determine liquid flow path and folded Kim Wipes which is used as an absorbent pad. (B) An Image of the assembled cellulose testing unit which is held together using paper binders. (C) An image of the top piece of the acrylic manifold which contains an opening to access the cellulose test strip. (D) An image of cellulose test strip. The strip was printed and cut as one piece. Wax ink was used for printing (black area) to define the liquid flow path. To create 3 layers of the test strip, the printed paper is folded in a zig-zag motion until the hydrophobic regions are aligned (inset). The top most layer of the cellulose paper contains a circular testing region (white, hydrophilic area without ink) with a diameter of 5 mm whereas the lower two layers, each, contain hydrophilic regions with a diameter of 6 mm. Each cellulose testing unit contains two test zones for testing of two reactions. (E) An image of the folded Kim Wipes which was folded in half for 6 times and used as absorbent material. (F) An image of the lower piece of the acrylic manifold which contain 3 mm spacers at two ends. The spacers are used to control consistent pressure that applies to different cellulose testing units. (G) An image of the light box with the phone placed at the top face. (H) An image of a slight opened light box showing phone fixture and a void for phone camera access at the top face. (I) An image of opened light box showing internal structure of the box. Warm-white LED light strips were attached to the inner top face of the light box to provide consistent lighting for all images. Cylindrical, white papers were covered parts of the LET strips to diffuse light from the strip, prevent shadow which may form on the cellulose test unit. Fixture was placed at the base of the light box to provide a fixed location to place the cellulose test unit.

FIG. 10. Overview of workflow and signal analysis of cellulose pull-down VNT. (A) Schematic of working principle and workflow of cellulose pull-down VNT (cpVNT). (i) Un-diluted plasma is mixed with receptor binding domain (RBD) tagged to cellulose binding domain (RBD-CBD) and angiotensin-converting enzyme 2 (ACE2) receptor tagged to biotin and streptavidin horse-radish peroxidase (ACE2-BA-SA-HRP). (ii) The reaction is incubated for 5 min to allow NAbs/RBD-CBD or ACE2-BA-SA-HRP/RBD-CBD complex formations. (iii) The mixture is applied on the cellulose-based vertical-flow device. (iv) Washing solution and ready-to-use 3,3′,5,5′-tetramethylbenzidine (TMB) substrate solution are sequentially applied to the device. Colorimetric reaction is allowed to develop for 3 min. Signals were captured using camera. (B) Representative images of cpVNT test results at different concentrations of NAb (mouse anti SARS-CoV-2 neutralizing antibodies) and non-NAb (human anti RBD non-neutralizing antibodies) spiked non-diluted human plasma. (C) Plots of inhibitory percentages of different antibody concentrations derived from cyan intensity signals on the cellulose test strips. % Inhibitions were calculated using the following formula:

$$\% inhibition = 1 - ( Inhibition \% = ( 1 - \frac{Cyan intensity of sample}{Cyan intensity of negative control ( no NAb )} ) * 100 \%$$

Limit of detections (LOD) were determined using mean+3SD formular and represented as the dotted lines. All data were represented as mean±SD. Each data points were performed in triplicates. 4 Parameters logistic model was used to draw the fitted curve with R² value of 0.9534.

FIG. 11. Comparison of COVID-19 NAb status from convalescents samples between pVNT and sVNT methods. Evaluation of NAb status from COVID-19 convalescent samples at different time points post infection from different follow up visits (FV) using (A and B) pVNT and (D and E) sVNT. Data shown in (A and D) were mean±SD of % neutralization for pVNT and mean±SD % inhibition for sVNT, respectively. Each data points were performed in triplicates. Lines connecting different data points represented samples that were obtained from the same subjected collected at different time points. Data shown in (B and E) were mean values of % neutralization for pVNT and % inhibition for sVNT from different samples. (C) Correlation between sVNT and pVNT. Mean values from each data point were represented on the graph. (F) Alternative correlation plot between sVNT and pVNT where data were arranged from highest to lowest values of pVNT neutralization percentages. Data were represented as mean±SD. Grey and black dotted lines represent cut-off values at 50% and 20% for pVNT and sVNT, respectively, to distinguish positive and negative NAb status.

FIG. 12. Evaluation of NAb status from COVID-19 convalescent samples, at different time points post infection, from different follow up 1.0 visits (FV) using cpVNT. (A) NAbs from all samples and visits as compared to pre-COVID plasma samples. Cut-off value which distinguished positive from negative NAb levels was designated at 20% inhibitory percentage (grey line). Each data point represented mean value of % inhibition of different samples. Each point was performed in triplicates. (B) NAbs detection using cpVNP from different samples at different time points. Data were represented as mean±SD. Each data point was performed in triplicates. Lines connecting different data points represented signals obtained from the same sample at different time points. (C) Alternative presentation of NAbs detected from different visits. Each data point represented mean value of % inhibition obtained from different samples. Each point was performed in triplicates. (D) Correlation between cpVNT and pVNT. Black and grey lines represent cut-off values at 20% and 50% for cpVNT and pVNT, respectively. Each data point represented mean value of % inhibition or % neutralization for cpVNT and pVNT, respectively. Each point was performed in triplicates. (E) Correlation between cpVNT and sVNT. Black and grey lines represent cut-off values at 20% for cpVNT and pVNT. Each data point represented mean value of % inhibitions for cpVNT and pVNT. Each point was performed in triplicates. (F) Cross reactivity test of cpVNT using antibodies against different viruses or viral antigens spiked in healthy plasma samples. Grey line represents a cut-off value at 20%. Each data point represented % inhibition from a single cpVNT experiment. Three separate experiments were performed for each condition.

FIG. 13. The cpVNT method enables shorter NAb detection time than current standards. Comparison between different VNTs including conventional VNT (cVNT) using live virus, pVNT using pseudovirus, sVNT using plate-based ELISA format and cpVNT using cellulose-based vertical flow device.

FIG. 14. RBD-CBD and mFc-ACE2 purifications and kinetic study and example of images obtained from light box and phone camera. (A) SDS-PAGE images of recombinant RBD-CBD, mFc-ACE2 receptor and N proteins expressed and purified for this study. (B) Bio-layer Interferometry (BLI) of biotinylated mFc-Ace2 receptor on streptavidin probes and different concentrations of RBD-CBD ranging from 6.25 nM-100 nM. K_{D} value was observed at 12.7 nM. (C) BLI of biotinylated mFc-Ace2 receptor on streptavidin probes with 100 nM RBD-CBD and N protein from SARS-CoV-2. (D) Examples of images obtained from the phone camera and the light box. Each cellulose testing unit was tested using different NAb concentrations spiked in healthy control plasma, including 0 nM in the left unit, 10 and 5 nM in the middle unit, and 100 nM in the right unit. Optimized cpVNT testing condition was used to perform these tests.

FIG. 15. Comparison of signals on cellulose-based vertical flow device using different test configurations. Inset depicts drawings of (a) CBD, (b) RBD, (c) RBD tagged with CBD (RBD-CBD), (d) ACE2 receptor conjugated biotin (ACE2-BA) and (e) HRP conjugated streptavidin (SA-HRP). (A) Different test configurations were tested to ensure that signals detected from RBD-CBD/ACE2-BA-SA-HRP complex were specific and integration of RBD-CBD promotes rapid signal detection. (B) Cyan intensities measured from each test configuration are in presented in a bar chart format. Based on the BLI data (FIG. 18B), >80% of RBD-CBD/ACE2 complex was formed within 300 sec (5 min). Therefore 300 sec incubation time were used to allow RBD-CBD/ACE2-BA-SA-HRP to form for the cellulose-based vertical flow device. (i) ACE2-BA-SA-HRP alone or with (ii) bare RBD or (iii) bare CBD produced equally low signals of cyan intensities. These results indicated that bare RBD alone could not be immobilized on the cellulose matrix within the 10 second flow-through time. In addition, CBD does not produce non-specific signal between ACE2-SA-HRP and CBD. (iv) Equivalent amount of RBD-CBD to the liquid phase premix conditions was immobilized on cellulose matrix. Data demonstrated that only slight increase in cyan intensity was observed as compared to the baseline value (shown in A). This data indicated that the 10 second flowthrough time was not sufficient to allow the complex formation. (v) In a similar configuration to (iv), longer incubation time between RBD-CBD and ACE2-BA-SA-HRP allowed significant increase in cyan intensity, indicating that the complex formed much more efficient with longer incubation time. However, pre-immobilizing of RBD-CBD on cellulose paper introduced complicated workflow to the assay in which solution has to be maintained on the cellulose surface for 5 min before it is allowed to flow pass the test spot. (vi) Due to the known property of CBD which can interact rapidly to cellulose matrix, the pre-mix condition was introduced to allow RBD-CBD/ACE2-BA-SA-HRP complex formation before the whole complex can be captured onto the cellulose paper. Cyan intensity from this configuration showed the highest value as compared to others, confirming that CBD can be captured rapidly onto the cellulose matrix. In addition, cyan intensity from condition (vi) has shown to be significantly higher than condition (v) (Student t-test, p-value of 0.00076), suggesting that the liquid phase incubation promoted more efficient complex formation as compared to the pre-immobilized capture reagent at equimolar concentration. Each data point was represented as mean±SD and was performed at least in triplicates.

FIG. 16. Optimization of RBD-CBD, ACE2-BA and SA-HRP concentrations for cpVNT. To optimize for reagent concentrations, plasma samples containing 0 or 100 nM NAb were used to determine the maximal and minimal cyan intensity signals. Concentrations of reagents that provide the highest ratio of max/min (0 nM:100 nM) signals were selected for the cpVNT. (A) Cyan intensity obtained from different RBD-CBD concentrations when ACE2-BA and SA-HRP concentrations were fixed at 20 nM and 2 nM, respectively. (B) Ratio of cyan intensity obtained from 0 nM:100 nM NAb. Highest ratio was observed from 10 nM of ACE2-BA, therefore this concentration was selected. Two different concentrations of RBD-CBD were tested including 10 and 20 nM. Concentrations of ACE2-BA and SA-HRP were fixed at 10 nM and 2 nM, respectively. The ratio between 0 nM:100 nM NAb of both RBD-CBD concentrations show similar value at ˜2.1, therefore different concentrations of NAb were tested to further inspect changes in cyan intensities. Cyan signals obtained from (C) 20 nM RBD-CBD showed minimal changes in the color intensities at low NAb concentrations whereas the signals obtained from (D) 10 nM RBD-CB showed distinguishable signals at low NAb concentrations, therefore nM RBD-CBD was selected for cpVNT. To optimize for SA-HPR concentration, RBD-CBD and ACE2-BA concentrations were fixed at 10 nM. (E) Cyan intensities obtained different concentrations of SA-HRP showed minimal changes at 100 nM NAb. More changes were observed from 0 nM NAb where 6 nM SA-HRP show the highest signal. (F) Ratios between 0 nM: 100 nM NAb obtained from different concentrations of SA-HRP. Highest ratio was observed at 6 nM SA-HRP, therefore this concentration was selected for cpVNT.

FIG. 17. Detection of IgG, IgA and IgM against SARS-CoV-2 Spike (S), receptor binding domain (RBD) and nucleocapsid (N) proteins using ELISA. Plasma samples were obtained from COVID-19 convalescent patients at different follow up visits (FV) post infections. FV1, 2, and 3 were ranged from 29-73 days, 82-129 days and 183-213 days, respectively. Each data point represented mean values of optical signal read at 450 nm. Each point was performed in triplicates. Overall mean±SD values from different samples from each visit were shown in dark grey lines.

FIG. 18. Assessment of IgG against different SARS-CoV-2 antigens. Assessment of IgG against (A) S, (B) RBD and (C) N proteins from plasma samples obtained from confirmed COVID-19 patients at different visits. Only samples from patients who came for the follow up visits were included in this analysis. Sample sizes for each of the follow up visit for FV1, FV2 and FV3 were 13, 13 and 10, respectively. Statistical analysis was done using student pair T-test. * indicates statistical difference at p value of 0.002. Each data point represented mean values of optical signal read at 450 nm. Each point was performed in triplicates. Lines connecting between different data point indicated that signals were obtained from the same sample from different visits.

FIG. 19. Analysis of sVNT performance using known concentrations of antibodies to define a cut-off inhibitory percentage. (A) Dose response curves of SARS-CoV-2 NAbs and hAnti-RBD non-neutralizing antibodies spiked in human plasma. Each data point represented mean±SD. Each point was performed at least in triplicates. 4 Parameters logistic model was used to draw the fitted curve with R² value of 0.9901. (B) Receive operating characteristic (ROC) curve and (C) sensitivity and specific of sVNT at different signal inhibitory percentages. At ˜20% inhibitory percentage, sVNT sensitivity maintains high at 75% and achieve specificity of 100%. As such, 20% cut-off is employed for sVNT.

FIG. 20. Correlation between NAbs measured using sVNT and IgG against different SARS-CoV2 proteins obtained from ELISA. Correlation of sVNT and IgG against (A) S, (B) RBD and (C) N proteins. (D) NAbs status from follow up visit 1 (FV1) determined using sVNT. Data were arranged in the order of highest to lowest values. Grey bars represent samples which exhibit moderate to severe symptoms whereas black bars represent samples which exhibit mild to no symptoms. Each data point in dot plots represented mean values of different samples. Each point was performed in triplicates. Data in bar chart were represented at mean±SD. Each point was performed in triplicates.

FIG. 21. Alternative correlation plots of cpVNT against pVNT and sVNT. (A) Correlation plot between pVNT and cpVNT with NAb status arranged from highest to lowest values. Grey and black lines represent cut-off values which determine positive and negative NAb status of pVNT and cpVNT at 50% and 20%, respectively. (B) Alternative correlation plot of sVNT and cpVNT with NAb status arranged from highest to lowest values. Black line represents a cut-off value for sVNT and cpVNT at 20%. All data points were represented as mean±SD. Each point was performed at least in triplicates.

FIG. 22. cpVNT test results obtained from human serum samples.

Inhibitory percentages derived from cyan intensity signals obtained from different concentrations of mouse anti SARS-CoV-2 neutralizing antibodies and human anti S1 antibodies that did not possess neutralizing property. Limit of detections (LOD) were determined using mean+3SD formular and represented as a dotted line. All data points were represented as mean±SD. Each point was performed at least in triplicates.

FIG. 23. Comparison chart between different VNTs.

FIG. 24. Schematic of RAPIDS process for screening of complementary binder pair.

FIG. 25. Bio-layer Inferometry (BLI) analysis of rcSso7d binders against SARS-CoV-2 N protein. (A and B) BLI results showed that Sso.E1 bound to CTD and Sso.E2 bound to NTD of the N protein, respectively and solely. (C) Results showed that only the combination of Sso.NP.E1/N protein/Sso.NP.E2 displayed stepwise increase in the binding curve, confirming that the binder pair bind to complementary epitopes on SARS-CoV-2 N protein.

FIG. 26. Assessment of different vertical flow assay (VFA) formats. (A) Four different assay approaches were assessed: 1) short incubation (SI) where capture molecule was immobilized on a cellulose surface and analyte and reporting molecules were applied to the cellulose paper in a stepwise manner, 2) limited incubation (LI) where capture molecule was immobilized on a cellulose surface, but analyte and reporter molecules were allowed for a short incubation period of 10 seconds prior loading to the cellulose test matrix, 3) half complex cellulose pulldown (CP-H) where capture molecule was immobilized on a cellulose paper while analyte and reporting molecules were allowed for interaction for a longer period of 1 minute prior loading onto the test matrix and 4) full complex cellulose pulldown (CP-F) where the interaction between capture molecule, analyte and reporting molecule was allowed in an aqueous phase for 1 minute prior loading onto the test matrix. (B) Results revealed that the CP-F test format produced the strongest signal intensity. (C) The CP-F test format also showed the highest ‘signal-to-noise’ ratio (+antigen/−antigen).

FIG. 27. Construction of control reaction. (A and B) The control reaction was constructed by pre-immobilizing the cellulose control spot with SARS-CoV-2 N protein. To immobilize the N protein on the cellulose surface, the protein was fused to CBD at its C terminus (NP-CBD). The NP-CBD on the cellulose control spot is designed to capture the reporting molecule (BA-MBP-Sso.E1) presented in the sample mixture, followed the CP-F test format. (C) The control spot performance was tested with pooled healthy control saliva samples spiked with either N protein or PBS. Results showed that the control spots produced cyan intensity signals higher than 0.2 for both testing conditions.

FIG. 28. Rapid SARS-CoV-2 vertical flow assay (VFA) workflow and performance. (A) Workflow of assay. (B) Dose response curve of different concentrations of N protein ranging from 0-50 nM spiked in saliva. Results showed that the VFA achieved a LoD at 2.5 nM. (C) Dose response curve of different SARS-CoV-2 concentrations spiked in saliva. Results revealed that the test can detect live virus at a concentration as low as 6.3×10⁴ TCID50/mL. (D) Cross-reactivity study of the rapid cellulose-based SARS-CoV-2 antigen test using different types of flu-causing pathogens spiked in saliva. All 18 pathogens had signal below the cut-off value (negative+3σ), while positive control with 4 nM SARS-CoV-2 N protein showed positive results.

FIG. 29. Translation of rapid SARS-CoV-2 VFA for Point-of-Care (PoC) applications. (A) In a first approach, a complementary ‘light box’ was created to ensure the consistency of lighting condition and the phone camera distance. (B) The software in the first approach utilizes phone camera to capture an image, followed by cyan intensity analysis and result interpretation. (C) With this approach, LoD of 2 nM SARS-CoV-2 N protein was achieved. (D) In a second approach, a spectrophotometer was designed to measure the red absorbance at 650 nm. (E) LoD of 4.0×10³ TCID50/mL virus was achieved using the custom-made spectrometer system.

FIG. 30. Images of purified proteins.

FIG. 31. Analysis of colorimetric signals obtained from control spots. (A) Evaluation of the performance of the control spots with different concentrations of N protein spiked in saliva matrix. (B) Evaluation of the performance of the control spots with different amount of SARS-CoV-2 virus spiked in saliva matrix. (C) Evaluation of the performance of the control spots with various pathogens spiked in saliva matrix.

FIG. 32. Images of test strip construction. (A). Schematic representation of cassette assembly; (B). Cassette assembled with plastic manifold and paper clips; (C). Cassette assembled with plastic manifold and double-sided tape; (D). Aluminum cassette assembled with screw.

FIG. 33. Assessment of washing step. (A). Test spot images of VFAs carried out with and without washing step and with or without 5 nM SARS-CoV-2 NP respectively; (B). Cyan intensity analysis for VFAs carried out with and without washing step; (C). Signal to noise ratio (S/N) analysis for VFAs carried out with and without washing step.

FIG. 34. Assessment of different colorimetric developmental times. (A). Test spot images of VFAs carried out with or without 5 nM SARS-CoV-2 NP that are developed for 1 or 2 or 3 minutes; (B). Cyan intensity analysis for VFAs carried out with or without SARS-CoV-2 NP for different development times; (C). Signal to noise ratio (S/N) analysis of VFAs that have been development for signal for 1 or 2 or 3 minutes.

FIG. 35. Analysis of colorimetric signals obtained from control spots of the PoC tests. (A). Cyan intensity at control spot with different concentrations (0, 1, 2, 3, 4, 5 nM) of SARS-CoV-2 NP spiked in saliva test matrix; (B). Absorbance (650 nm) at control spot using Attonics spectrophotometer absorbance reader system with different concentrations (0, 0.032, 0.16, 0.8, 4, 20, 1,000×10³ TCID₅₀/mL) of SARS-CoV-2 virus spiked into saliva test matrix.

FIG. 36. Test principle and results of the rapid cellulose-based SARS-CoV-2 test that detect NAb from non-processed whole blood samples. (A) The modified assay workflow which comprises two incubation steps. (B) Simplified schemes represent assay reaction on the ‘Test’ and ‘Control’ spots of the vertical flow device. (C) Fluorescent intensities obtained from different concentrations of NAb in non-infected, non-vaccinated whole blood samples. (D) % Inhibition of fluorescent signals derived from FIG. 3B. % Inhibitions were calculated using the following formula:

$$\% inhibition = ( 1 - \frac{Fluorescent intensity of sample - Baseline fluorescent intensity}{Fluorescent intensity of negative control ( no NAb ) - Baseline fluorescent intensity} ) * 100 \%$$

$$* Baseline fluorescent intensity was obtained from ACE 2 - FI applied on the paper without R BD - CBD .$$

FIG. 37. Results obtained from the rapid cellulose-based SARS-CoV-2 NAb test. Inhibition percentages of (A) all samples representing different stages of COVID-19 vaccination (B) samples received BNT 162b2 vaccine from Pfizer and (C) samples received mRNT-1273 vaccine from Moderna. % Inhibitions were calculated using the following formula:

$$\% inhibition = ( 1 - \frac{Fluorescent intensity of sample - Baseline fluorescent intensity}{Fluorescent intensity of negative control ( no NAb ) - Baseline fluorescent intensity} ) * 100 \%$$

$$* Baseline fluorescent intensity was obtained from ACE 2 - FI applied on the paper without R BD - CBD .$$

FIG. 38. Results obtained from RBD variants. (A) Fluorescent intensities obtained from non-infected and non-vaccinated whole blood samples tested on different RBD-CBD variants capture reagents. (B) Inhibition percentages of fully vaccinated whole blood samples tested on different RBD-CBD variants. % Inhibition was calculated using the following formula:

$$\% inhibition = ( 1 - \frac{Fluorescent intensity of sample - Baseline fluorescent intensity}{Fluorescent intensity of WT negative control ( no NAb ) - Baseline fluorescent intensity} ) * 100 \%$$

$$* Baseline fluorescent intensity was obtained from ACE 2 - FI applied on the paper without R BD - CBD .$$

### EXAMPLES

### Example 1—Comparison Between a Conventional Rapid Diagnostic Test (RDT) Format and a Cellulose Pull Down Assay Format

FIG. 1 shows a scheme of a conventional sandwich complex 100 for analyte detection. The conventional sandwich complex 100 comprises a target analyte 102 captured between a reporter agent 104 and a capture agent 106.

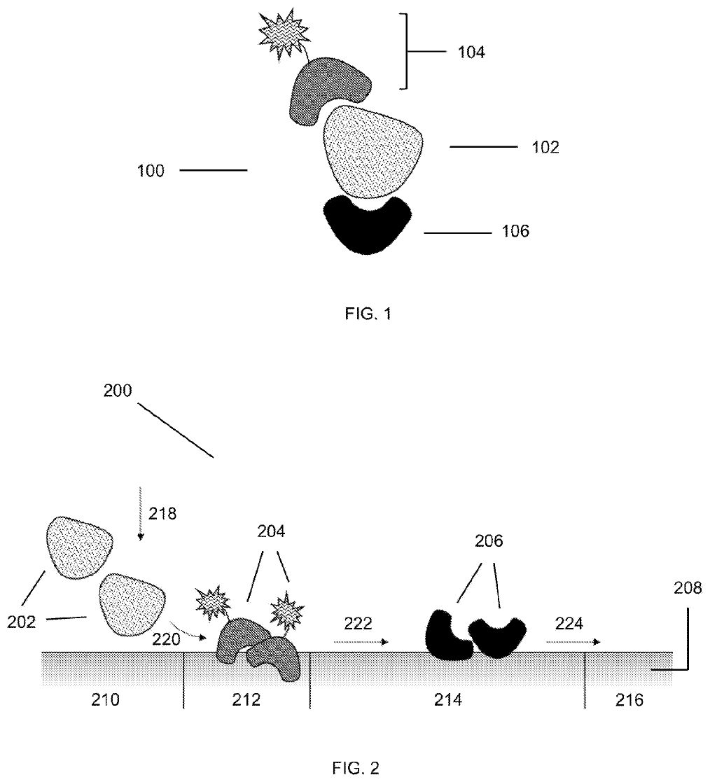

FIG. 2 shows a conventional rapid diagnostic test (RDT) format 200 using a cellulose substrate 208. A sample fluid containing analytes 202 is applied onto a sample loading pad 210 in a sample loading step 218. The sample fluid containing analytes 202 then travel under capillary action in step 220 to reach reporter agent pad 212 containing reporter agents 204. The analytes 202 come into contact with the reporter agents 204, which bind to the analytes 202. The sample fluid now containing reporter agent-bound analytes 202 continue to flow through the cellulose substrate 208 in step 222 to reach test zone 214 containing immobilized capture agents 206. Here, the capture agents 206 bind to the reporter agent-bound analytes 202 and immobilize them, forming a sandwich complex for signal detection at test zone 214. After the test zone 214, in step 224, the sample fluid is absorbed by waste pad 216 which facilitates consistent sample flow across the test zone 214.

The conventional RDT format suffers from several shortcomings. In the conventional paper-based RDTs, the reporter agents have to be dry stored on the cellulose substrate or other types of substrate used as reporter agent pad 212 and the capture agents have to be immobilized to the test zone, commonly cellulose substrate, prior to performing the assay. Analytes are brought to the reporter agents and capture agents via the capillary force. Once they reach the reporter agent pad, the analytes have less than 10 seconds to form half sandwich complexes with the reporter agents. The half sandwich complexes are then brought to the test zone to form a full sandwich complex with the immobilized capture agents. Here, the capture agents only have a few seconds to capture the half sandwich complexes. With the limited interaction time, most half sandwich complexes flow past the test zone without being captured. The inefficient formation of full sandwich complexes results in low assay sensitivity and potentially false negative results.

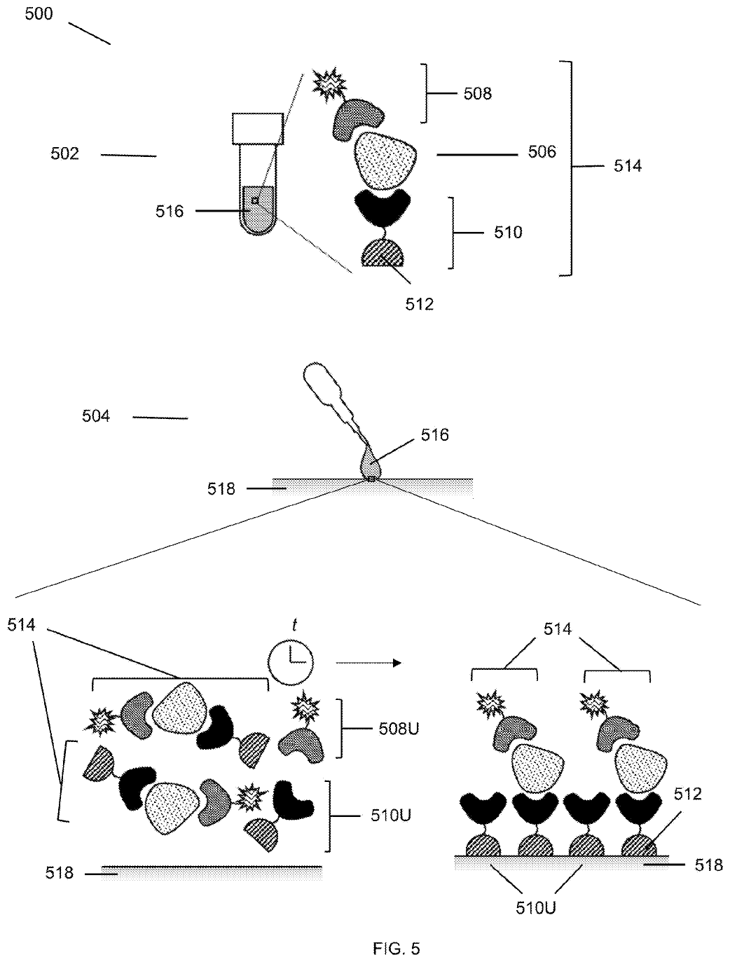

Furthermore, the immobilization of capture agents onto the cellulose substrate occurs spontaneously through hydrophobic and electrostatic interactions. These interactions offer only moderate strength, and thus a substantial amount of capture agents is lost through this process. In addition, this process happens without control over the orientation of the capture agents. As a result, not all of the capture agent binding faces are oriented towards the sample solution, as shown in FIG. 4A, further contributing to the low assay sensitivity.

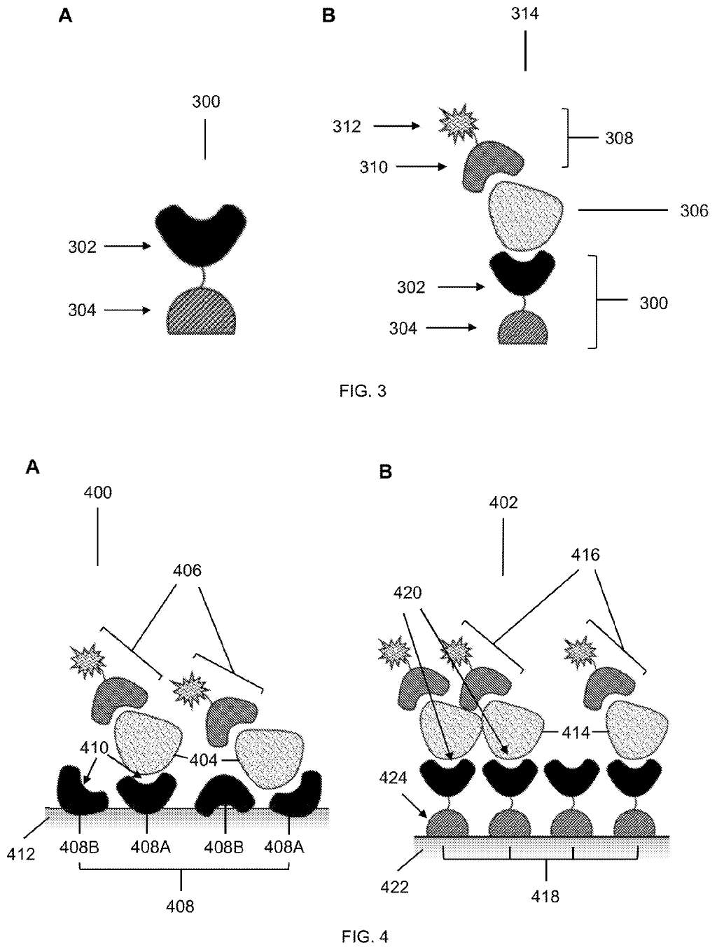

FIG. 3 shows a scheme of a capture agent 300 (FIG. 3A) and a sandwich complex 314 (FIG. 3B) in accordance with embodiments disclosed herein. The capture agent 300 comprises a cellulose binding domain (CBD) 304 that may be fused to an analyte binder 302. In a sandwich complex 314, a target analyte 306 is captured between the capture agent 300 comprising a CBD 304 and a reporter agent 308. The reporter agent 308 may comprise an analyte binder 310 coupled to a label 312. CBD is a protein domain that exists in many carbohydrate-active enzymes. It has very high affinity towards cellulose substrate in which interaction between the CBD and the cellulose substrate can happen in less than a second. The incorporation of CBD in the capture agent facilitates efficient capture of full sandwich complex to a cellulose substrate.

FIG. 4 shows a comparison between a conventional RDT format 400 and a cellulose pull down assay 402 format in accordance with an embodiment disclosed herein. FIG. 4A shows a conventional RDT format 400. Capture agents 408 are immobilized on the cellulose substrate 412 in random orientations, resulting in non-uniform directions of the binding surfaces 410. Only the capture agents 408A with binding surfaces 410 facing the correct directions are able to bind the analytes 404 to form a sandwich complex together with the reporter agents 406 bound to the analytes 404. A substantial fraction of the capture agents 408B with binding surfaces 410 facing the incorrect directions are not able to capture the reporter agent-bound analytes 404, resulting in a resource waste of these capture agents. Only a limited amount of analytes 404 can be captured per area of the cellulose substrate 412, and hence the signal that may be generated from the analyte-bound reporter agents 406 is also limited. This increases the incidences of false negative results and reduces the sensitivity of the assay. FIG. 4B shows a RDT format 402 in accordance with an embodiment disclosed herein. The CBDs 424 of the capture agents 418 bind to the cellulose substrate 422, orienting the binding faces 420 of the capture agents 418 in a uniform direction towards the solution containing analytes 414. A large fraction of the capture agents 418 can bind successfully to the analytes 414. As a result, more analytes 414 can be captured per area of the cellulose substrate 422 and signal from the analyte-bound reporter agents 416 is enhanced. This reduces the incidences of false negative results and increases the sensitivity of the assay. A resource waste of the capture agents is also minimized.

FIG. 5 shows a scheme of a cellulose pull down full complex method 500 in accordance with an embodiment disclosed herein. In step 502, analytes 506, reporter agents 508 and capture agents 510 comprising CBDs 512 are incubated in solution 516 to form full sandwich complexes 514 off-site of a cellulose substrate. This step allows sufficient interaction time between the analytes 506 and the reagents 508 and 510, thereby promoting effective formation of the full sandwich complexes 514. Typically, the full sandwich complexes 514 are formed within 1-3 minutes. Once the full sandwich complexes 514 are formed, the pre-incubated solution 516 containing the full sandwich complexes 514 (and any unbound analytes, unbound reporter agents, unbound capture agents and half sandwich complexes (not depicted) e.g. reporter agent-bound analytes and capture agent-bound analytes) are applied to a cellulose substrate 518 in step 504. The contents of the solution 516, including sandwich complexes 514 and any unbound reporter agents 508U and unbound capture agents 510U come into contact with the cellulose substrate 518. The interaction between the CBDs 512 and the cellulose substrate 518 is rapid. Within a period of time t (which can be as little as within seconds), the full sandwich complexes 514 become captured onto the cellulose substrate 518 via affinity between the CBDs 512 of the full sandwich complexes 514 and the cellulose substrate 518. Any unbound capture agents 510U and capture agent-bound analytes (not depicted) may also be captured onto the cellulose substrate 518. Presence of analytes 506 are detected by detecting the presence of full sandwich complexes 514 immobilised on the cellulose substrate 518. Embodiments of the cellulose pull down assay method as disclosed herein allows more interaction time between the analytes and reagents, thereby increasing effective formation of full sandwich complexes. Further, because of the high interaction affinity between CBD and cellulose, most of the complexes can be captured onto the cellulose paper, with the capture occurring within seconds. As a result, assay sensitivity is enhanced and false negative results are reduced with minimal resource waste (e.g. unbound reporter agents and unbound capture agents).

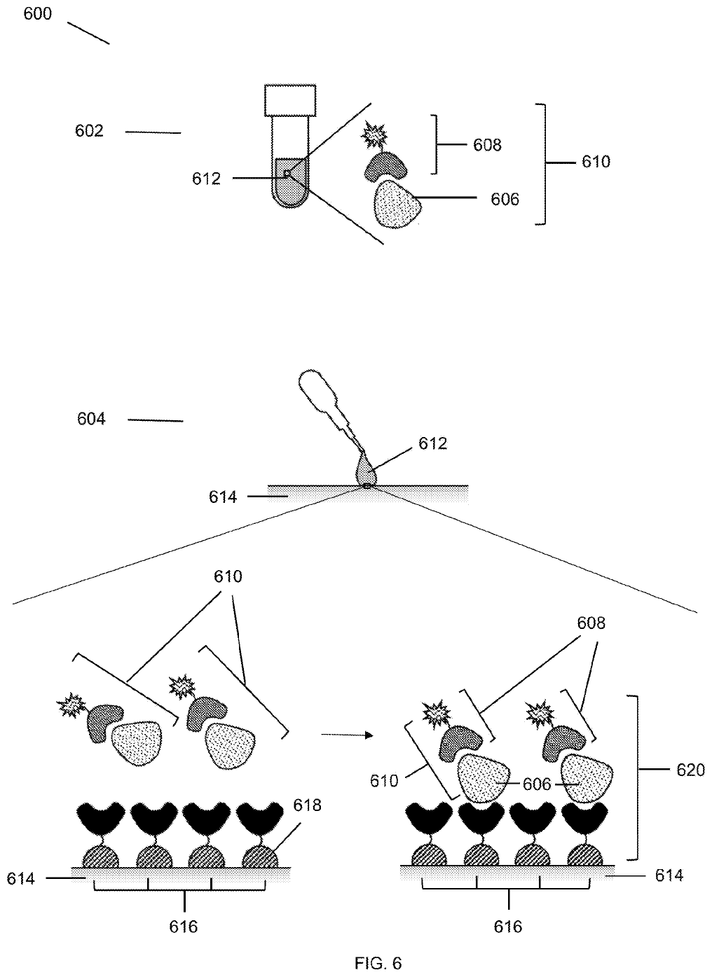

FIG. 6 shows a scheme of a cellulose pull down half complex method 600 in accordance with an embodiment disclosed herein. Certain paper-based RDT devices may come in formats that are not compatible with the pre-incubation of full sandwich complexes, for example, devices that contain multiple layers of cellulose paper where the test zone is embedded inside the multi-layered paper, or devices that require cellulose substrate for fluidic path, etc. For such devices, application of a pre-incubated solution containing pre-formed full sandwich complexes may result in an un-desirable capture of the complexes on the first layer, or on the loading port of the cellulose entry point, due to the CBD-cellulose interaction. For such devices, the solution can be pre-incubated to form half sandwich complexes, instead of full sandwich complexes, and then applied to a cellulose substrate containing immobilised capture agents in the desired test zone(s) in accordance with embodiments of the method 600. In an embodiment of the method 600, in step 602, analytes 606 are incubated with reporter agents 608 in solution 612 to form half sandwich complexes (i.e. reporter agent-bound analytes) offsite of a cellulose substrate. This step allows sufficient interaction time between the analytes 606 and the reporter agents 608, thereby promoting effective formation of the half sandwich complexes 610. Typically, around 1-3 minutes is sufficient for the formation of the half sandwich complexes 610. Upon formation of the half sandwich complexes 610, the pre-incubated solution 612 containing the half sandwich complexes 610 (and any unbound analytes and unbound reporter agents (not depicted)) are applied to a cellulose substrate 614 in step 604. The cellulose substrate 614 comprises capture agents 616 that are pre-immobilised thereon e.g. at the desired test zones. The capture agents 616 may each contain a CBD 618 having affinity for the cellulose substrate 614. During the application step 604, the contents of the solution 612, including half sandwich complexes 610 and any unbound reporter agents (not depicted) come into contact with the capture agents 616 on the cellulose substrate 614. The capture agents 616 capture the half sandwich complexes 610 to form full sandwich complexes 620 on the cellulose substrate 614. Presence of analytes 606 are detected by detecting the presence of full sandwiches complexes 620 immobilised on the cellulose substrate 614. Embodiments of the cellulose pull down assay method as disclosed herein allows more interaction time between the analytes and reporter agents, thereby increasing the effective attachment of the analytes to the detectable reporter agents. This may promote high sensitivity signal production form the reporter agents. In addition, the incorporation or fusing of CBD to the capture agents promote homogenous orientation of the capture agents by direct the analyte binding faces toward the solution (FIG. 4B), thereby facilitating effective capture of the analytes and further enhancing high sensitivity signal production. As a result, assay sensitivity is enhanced and false negative results are reduced with minimal resource waste (e.g. unbound reporter agents and unbound capture agents).

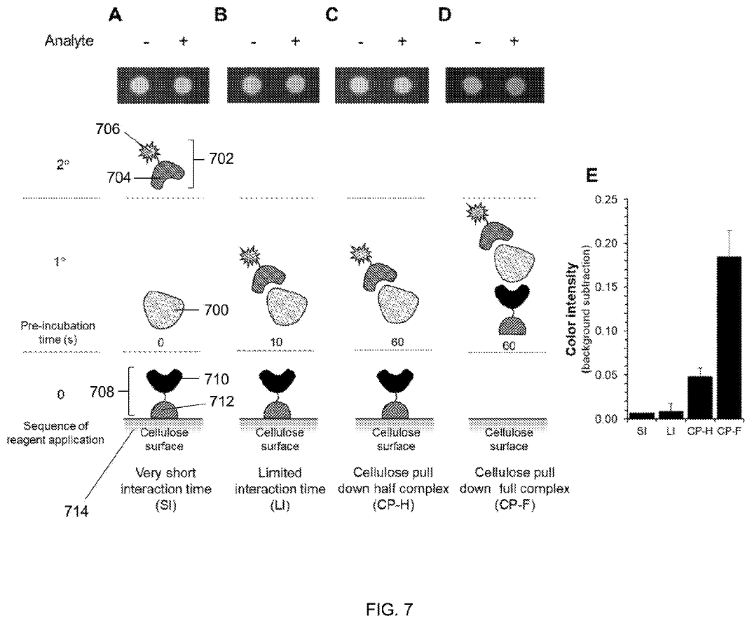

FIG. 7 shows the colorimetric results of different interaction times between the analyte 700 and the reagents (reporter agent 702 comprising an analyte binder 704 coupled to a label 706 and capture agent 708 comprising an analyte binder 710 fused to a CBD 712). The experiments were performed on cellulose papers 714. The conditions tested are the following: (A) very short incubation time (SI), (B) limited incubation time (LI), (C) sufficient incubation time for formation of half sandwich complex and pull down using pre-immobilized capture agent comprising CBD (i.e. cellulose pull down half complex—CP-H) and (D) sufficient incubation time for formation of full sandwich complex and pull down using high interaction affinity between CBD and cellulose substrate (i.e. cellulose pull down full complex—CP-F). The results demonstrate the proof-of-concept and also the advantages of embodiments of the method as compared with the approach where only limited interaction time is allowed on the cellulose substrate/paper for formation of complexes. Negative and positive samples refer to absence or presence of the target analyte. Results from FIG. 7A show that the sequential application of different reagents (i.e. very short interaction time, SI) produced minimal distinguishable colorimetric signal between negative and positive samples (FIG. 7E). FIG. 7B represents a condition where only limited interaction time (LI) is allowed on a cellulose substrate/RDT device. In this condition, minimal colorimetric signal difference is observed between the negative and positive samples. Results from these experiments indicate that full sandwich complexes are not formed and/or not captured effectively on to the substrate/paper (FIG. 7E). In FIG. 7C, cellulose pull down (CP) technology is applied using half complex pre-incubation (CP-H) in accordance with an embodiment disclosed herein. As compared to LI condition (FIG. 7B), the pre-incubation time is increased from 10 seconds to 1 minute, allowing sufficient interaction time for half complex formation. For this condition, distinct colorimetric signals from the two samples can be visually noticed, with darker blue colorimetric signal being observed from the positive samples (FIG. 7E). In FIG. 7D, CP with full sandwich complex (CP-F) is carried out by pre-incubating the reagents and the samples for the formation of full sandwich complexes in accordance with an embodiment disclosed herein. The pre-incubation time is 1 min off-site of the cellulose paper. The results show obvious blue colorimetric signals from the positive samples as compared to the negative samples (FIG. 7E). Results from CP-H and CP-F indicate that with sufficient pre-incubation time and CBD, full sandwich complexes are effectively formed and captured on to the cellulose paper. Results from CP-F also suggest that CBD can be captured rapidly and effectively onto the paper, therefore enhancing further the assay sensitivity. Embodiments of the methods CP-H and CP-F were observed to give 7 and 26 times increase in colorimetric signal production respectively, as compared to SI assay format. The increase in signal production indicates improved capture efficiency of full complex on to the cellulose paper. With this improvement, embodiments of the methods are expected to advantageously lower the Limit of Detection (LoD) of biomarkers when applied in RDT.

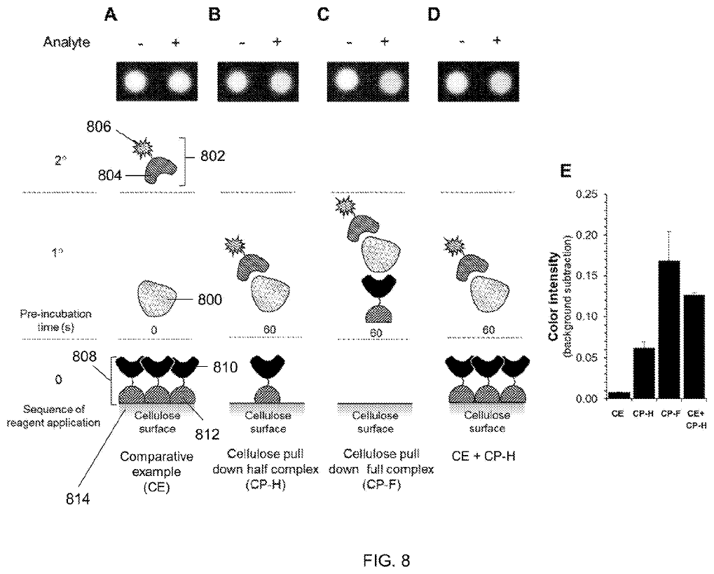

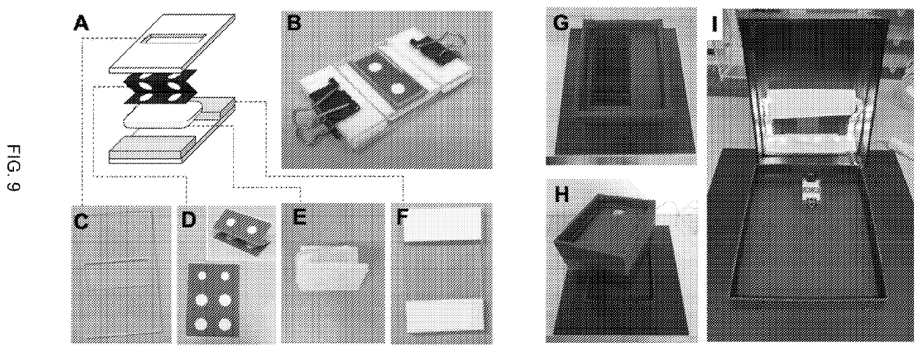

FIG. 8 shows the colorimetric results obtained from varying the interaction time between the analyte 800 and reagents (reporter agent 802 comprising analyte binder 804 coupled to a label 806 and capture agent 808 comprising an analyte binder 810 fused to a CBD 812) and/or the concentration of the capture agent 808. The experiments were performed on cellulose papers 814. The conditions tested are the following: (A) an assay performed according to a comparative example (CE) where a high molar concentration of capture agents comprising CBD were immobilized on the cellulose substrate and other reagents were applied sequentially as per standard immunoassay protocol, (B) cellulose pull down half complex (CP-H) where 1 minute pre-incubation for formation of half sandwich complexes is performed before addition to the test zone of the substrate that contain capture agents comprising CBD. The capture agent concentration is 8 times lower than the CE test format. (C) cellulose pull down full complex (CP-F) where 1-minute pre-incubation for formation of full sandwich complexes is performed before adding to the test zone. The capture agent concentration is 8 times lower than the CE test format. (D) Combination of the CE and CP-H where high molar concentration of capture agents comprising CBD were present at the test zone and half sandwich complexes were allowed to be formed by 1-minute pre-incubation before adding to the test zone. The results show the superior performance of cellulose pull down (CP) assays in accordance with the embodiments disclosed herein as compared to the comparative example. The comparative example is based on a previous concept of employing high molar concentration of capture agents on the cellulose substrate to improve capture efficiency of the target analytes. The high molar concentration of capture agents is allowed to be uniformly immobilised on the cellulose substrate via the CBD of the capture agents. Follow this principle, 2 μL of 20 μM (40 pmol) CBD-tagged capture agent was immobilised onto cellulose paper and antigen detection assay was carried out following standard immunoassay protocol, namely to sequentially add different reagents ((i) analyte, (ii) reporter agent, and (iii) colorimetric signal producing reagent) on to the cellulose substrate (FIG. 8A). Along-side this assay, CP-H and CP-F assays were performed by using 8 times less (5 μmol) CBD-tagged capture agents. For the CPH and CP-F assays, 1 minute incubation time is carried out off-site of the cellulose test zone (half sandwich complex for the CP-H and full sandwich complex for the CP-F) to allow all reagents to interact before applying them to the test zones. Results showed that both CP-H and CP-F produced significantly higher signals than the comparative example (CE) by 8 (FIGS. 8B and E) and 22 times (FIGS. 8C and E), respectively. Additional experiments to combine the method of the comparative example with the CP-H method disclosed herein and it was found that, by allowing for a 1 minute incubation for the formation of half sandwich complexes instead of sequential application, the signal can be improved by 17 times as compared to the signal observed from the CE assays (FIGS. 8D and E).

The low concentration of capture agents used in CP-H, CP-F and the hybrid method CP-H+CE is not expected to improve assay sensitivity. However, the results surprisingly indicate that the combination of (i) the reagents pre-incubation and (ii) the capture agents comprising CBD, allows the low concentration of the capture agents to effectively capture the full sandwich complexes and be efficiently pulled down to the cellulose substrate (in CPF). Embodiments of the method disclosed herein advantageously improve assay sensitivity and minimise resource waste i.e. use of capture agents and reporter agents are optimised.

In the comparative example, a high concentration of capture agent at 18 μM was required to be deposited on cellulose substrate to ensure efficient capture of analytes-reporter agents complex in sequential matter. Although analytes can typically be captured efficiently and rapidly using high molar concentration of capture agent, without being bound by theory, it is believed that the sequential application of reporter agents in the comparative example hinders the high sensitivity signal production because the reporter agents have short resident time to interact with the analytes on cellulose substrate. As a consequence, low sensitivity of the assay and false negative results are observed since the binding of the analytes and the reporter agents does not happen within seconds.

### Example 2—Cellulose Pull Down of SARS-CoV-2 Nucleocapsid Protein (NP)

### Engineering of Capture Agent (or Cbd Tagged Capture Reagent)

rcSso7d binder proteins that bind specifically to SARS-CoV-2 nucleocapsid protein (NP) were engineered using directed evolution approach. In brief, a library comprising theoretically 10⁹ variants of rcSso7d was generated. Each variant was cloned into a yeast surface display vector pCTcon2 and transformed into yeast S. cerevisiae. Recombinant SARS-CoV-2 N protein immobilized on magnetic particles were introduced to the rcSso7d yeast library. Yeast clones that bound to magnetic particles were sorted using magnetic stand and cultured to amplify the yeast cell number. His-tagged SARS-CoV-2 N protein was introduced to the yeast cells amplified from magnetic bead sorting. The SARS-CoV-2 N protein was stained using anti-His antibodies conjugated to fluorophore. Yeast cells that bound to SARS-CoV-2 and carried fluorescent antibody were sorted using FACS and cultured to amplify the yeast cell number. FACS sorting were repeated for 5 more rounds, each round with lower concentrations of SARS-CoV-2 N protein. Following 6 rounds of FACS sorting, rcSso7d sequences were sequenced and subcloned into bacterial expressing vector pET28b for protein expression in bacterial cells.

The pair of engineered rcSso7d proteins that bind to different epitopes of SARS-CoV-2 N protein were used as capture and reporter reagents for all proof-of-concept studies in this disclosure. The DNA sequence encoding CBD (Table 1) was tagged to the C-terminus of the capture reagent using standard restriction enzyme and ligation methods. In brief, the DNA sequence encoding CBD that contains (i) BamHI restriction enzyme sequence and a 3×GS spacer sequence (GGA GGT GGA GGT TCT GGT GGA GGA GGA TCT GGA GGT GGT GGT TCT) at its N-terminus and (ii) XhoI restriction enzyme sequence at its C-terminus was synthesized by a commercialized service provider and cloned into His-pET28b vector using indicated restriction enzyme sites and T4 ligase (New England BioLabs®, Inc., USA). Cloning of CBD into His-pET28b vector created a His-CBD-pET28b vector template for the subsequent cloning of capture reagent. To tag CBD to capture reagent, the DNA sequence encoding capture reagent that contains (i) NdeI restriction enzyme sequence at its Nterminus and (ii) BamHI at its C-terminus, was amplified (using Phusion high-fidelity DNA polymerases, ThermoFisher Scientific, USA) and cloned into His-CBD-pET28b vector using indicated restriction enzyme sties and T4 ligase. This process created His-rcSso7d_{SARS-CoV-2-NP}capture-pET28b vector that will be used to generate the ‘capture reagent’. All restriction enzymes and T4 ligase were obtained from New England BioLabs®, Inc., USA. The procedure for restriction enzyme digestion and ligation were performed as per manufacturer recommendation.

TABLE 1
CBD DNA and protein sequences

| | |
|---|---|
| CBD | CCGGTATCAGGCAATTTGAAGGTTGAATTCTACAACAGC |
| DNA | AATCCTTCAGATACTACTAACTCAATCAATCCTCAGTTC |
| sequence | AAGGTTACTAATACCGGAAGCAGTGCAATTGATTTGTCC |
| | AAACTCACATTGAGATATTATTATACAGTAGACGGACAG |
| | AAAGATCAGACCTTCTGGTGTGACCATGCTGCAATAATC |
| | GGCAGTAACGGCAGCTACAACGGAATTACTTCAAATGTA |
| | AAAGGAACATTTGTAAAAATGAGTTCCTCAACAAATAAC |
| | GCAGACACCTACCTTGAAATAAGCTTTACAGGCGGAACT |
| | CTTGAACCGGGTGCACATGTTCAGATACAAGGTAGATTT |
| | GCAAAGAATGACTGGAGTAACTATACACAGTCAAATGAC |
| | TACTCATTCAAGTCTGCTTCACAGTTTGTTGAATGGGAT |
| | CAGGTAACAGCATACTTGAACGGTGTTCTTGTATGGGGT |
| | AAAGAACCC |
| CBD | PVSGNLKVEFYNSNPSDTTNSINPQFKVTNTGSSAIDLS |
| protein | KLTLRYYYTVDGQKDQTFWCDHAAIIGSNGSYNGITSNV |
| sequence | KGTFVKMSSSTNNADTYLEISFTGGTLEPGAHVQIQGRF |
| | AKNDWSNYTQSNDYSFKSASQFVEWDQVTAYLNGVLVWG |
| | KEP |

### Engineering of Reporter Reagent (or Analyte Binder or Binding Protein)

To allow the reporter reagent to generate colorimetric signal, the reporter reagent was tagged to biotin acceptor (BA) sequence (GGC CTG AAC GAT ATT TTT GAA GCG CAG AAA ATT GAA TGG CAT GAA). In brief, DNA sequence encoding NdeI-BA3×GS_linker-EcoRI was synthesized by a commercialized service provider and cloned into HispET28b vector via NdeI and EcoRI restriction enzyme sites using T4 ligase. This process generated His-BA-pET28b vector. DNA sequence encoding maltose binding protein (MBP) that contains EcoRI sequence at its N-terminus and BamHI at its C-terminus was amplified and cloned into His-BA-pET28b at the indicate restriction enzyme sites using T4 ligase. This process created His-BA-MBP-pET28b vector. To prepare ready-to-used vector template with a spacer protein after MBP, DNA sequence encoding 3×GS_linker with BamHI and SpeI restriction enzyme sequences at its N- and C-terminus was synthesized and cloned into His-BA-MPB-pET28b vector using T4 ligase. Finally, the rcSso7d reporter reagent sequence containing SpeI and XhoI at its N and C terminus was amplified and cloned into the His-BA-MBP-pET28b vector using T4 ligase. This process created His-BA-MBP-rcSso7d_{SARS-CoV-2-NP-reporter}-pET28b vector which will be used to generate the ‘reporter agent’.

### Expression and Purification of Capture and Reporter Agents

In brief, the pET28b plasmids containing capture reagent tagged with CBD or reporter reagent tagged with BA were transformed to BL21-DE3 cells, plated on LB-agar plate containing 50 μg/mL of kanamycin and incubated for 12-18 hr at 37° C. A single colony was selected from each plate and inoculated in 10 mL LB media containing 50 μg/mL of kanamycin at 37° C. on a shaker for 12-18 hr. Cells were passage into a larger volume of 0.5-1 L of LB media containing kanamycin and cultured at 37° C. on a shaker until they reach log phase growth (˜OD of 0.6 at 600 nm). Protein expression was induced using 0.5 mM of Isopropyl β-D-1-thiogalactopyranoside (IPTG). For the reporter reagent 0.3 mM biotin was also supplemented to the culture. The cells were continued to be cultured at 20° C. on a shaker for 16-20 hr.

Cells were harvested by centrifugation at 4,000 g for 10 min. The cell pellets were re-suspended in binding buffer (50 mM Tris, 300 mM NaCl, 10 mM imidazole, pH 7.6) at a ratio of 1 g/10 mL and lysed by sonicating at 50% amplitude with 5 s on 10 s off cycles for 5 min. The lysed cells were centrifuged at 20,000 rpm for 30 min to remove cell debris. To purify the proteins (capture/reporter reagents), supernatants were collected and incubated with nickel resin (Nuvia IMAC Resin, BIORAD, USA) at a ratio of 1 mL resin/1 mL culture for 2-3 hr at 4° C. This step allows His-tagged proteins to be captured to the nickel resin, isolating the desired protein from other endogenous background proteins. The supernatant containing nickel resin bound to His tagged proteins was flown through a column where the nickel resin would be trapped in the column and excess liquid would flow pass the column. With this column, the resin was washed twice, each with 10 mL of binding buffer. The proteins were eluted using binding buffer containing 500 mM of imidazole. The eluted proteins were buffer exchanged to phosphate buffer saline (PBS) using Amicon® Ultra Centrifugal Filters (Merck, Singapore).

### Generation and Preparation of the Cellulose Paper-Based Matrix

Cellulose paper (Whatman No. 1) was printed with wax ink (Xerox ColorCube, Xerox, USA) to create a 2.5 mm hydrophilic test zone and baked at 150° C. for 1 min. An 11.4 cm×21.6 cm Kimwipes paper (Kimberly-Clark Professional™, Singapore) was folded in half for 4 times and placed underneath the printed cellulose test zone to form an absorbent pad. The cellulose test zone and the absorbent pad were kept in tight contact using 2 binder clips. For all experiments, the test zones were blocked with 10 μL of 5% bovine albumin serum (BSA) (Sigma Aldrich, Singapore) in PBS. All other reagents were prepared in 10% human serum in PBS. CBD tagged rcSso7d SARS-CoV-2 N protein capture reagent and BA tagged rcSso7d SARS-CoV-2 N protein reporter reagent were used in all experiments.

### Paper-Based Colorimetric Assays (FIG. 7)

For very short incubation time (SI) test format, 10 μL of each of the following reagents was applied to the test zone sequentially (i) 500 nM capture reagent, (ii) 0 or 50 nM of SARS-CoV-2 N protein, (iii) 500 nM of reporter reagent, (iv) 250 pM of streptavidin horse radish peroxidase (SA-HRP) (Biolegend, USA) and (v) ready-to-use 3,3′,5,5′-Tetramethylbenzidine (TMB) substrate (Sigma Aldrich, Singapore). All reagents were applied soon after the previous reagent was fully absorbed away from the test zone.

For the limited incubation time (LI) test format, 10 μL of 500 nM capture reagent was added to the test zone. Subsequently, 10 μL of reagent containing mixture of (i) 0 or 50 nM SARS-CoV-2 N protein, (ii) 500 nM reporter reagent and (iii) 250 pM SA-HRP was incubated for 10 sec at room temperature. Following the incubation, the mixture reagent was applied to the test zone. Finally, 10 μL of TMB was applied to generate colorimetric signal.

For half complex cellulose pull-down (CP-H) test format, 10 μL of 500 nM capture reagent was added to the test zone. For the subsequent steps, similar protocol to LI test format was carried out. However longer, 1 min, incubation time was allowed to incubate the mixture reagent before the reagent was added to the test zone. Finally, 10 μL of TMB was added to generate colorimetric signal.

For full complex cellulose pull-down (CP-F) test format, 10 μL of mixture reagent containing (i) 500 nM capture reagent, (ii) 0 or 50 nM SARS-CoV-2 N protein, (iii) 500 nM reporter reagent and (iv) 250 pM SA-HRP was incubated at room temperature for 1 min before applying to the test zone. Subsequently, 10 μL of TMB was applied to the test zone to generate visible signals. For all test formats, the cellulose test strips were allowed to develop colorimetric signals for 3 min before images were captured.

### Comparison of the Current Cellulose-Pull Down Assay Performance to a Comparative Example (FIG. 8)

To perform the assay in the comparative example (CE), 2 μL of 20 μM capture reagent (40 pmol) was added to the test zone to immobilize high molar concentration of capture reagent. Afterward, sequential application of the following reagents was carried out (i) 10 μL of 0 or 50 nM SARS-CoV-2 NP, (ii) 10 μL of 500 nM reporter reagent, (iii) 10 μL of 250 pM SA-HRP and (iv) 10 μL of TMB. The CP-H and CP-F assays were carried out as described earlier. The combination of CE and CP-H was done by immobilizing the cellulose test zone with 2 μL of 20 μM capture reagent. Subsequently, a mixture of the following reagents was prepared in a 10 μL solution: (i) 0 or 50 nM SARS-CoV-2 N protein, (ii) 500 nM reporter reagent and (iii) 250 pM SAHRP. The mixture was allowed to incubate at room temperature for 1 min before it was applied to the cellulose test zone. Finally, 10 μL of TMB was applied to the test zone to generate colorimetric signal. All tests were allowed to develop colorimetric signals for 3 min before images were captured.

### Image Analysis

Images were taken by phone camera or scanner and stored in .jpg format. The color intensities were analyzed using ImageJ software by measuring cyan values in CYMK color format.

For all applications described in the examples that follow, cellulose-based vertical flow device was used to host chemical reactions and capture the desire target analytes. The cellulose test strips were constructed by placing one or several layers of the cellulose paper above absorbent pads. Absorbent pads can be any porous materials that possess liquid absorbing properties. The cellulose test paper(s) and the absorbent pads were secured together using pressure applied by various means e.g., paper binders, chamber, cassettes, manifold, etc.

### Example 3—a Rapid Simple Point-of-Care Assay for the Detection of SARS-CoV-2 Neutralizing Antibodies

Neutralizing antibodies (NAbs) prevent pathogens/virus from infecting host cells. Determination of SARS-CoV-2 NAbs are critical to evaluate herd immunity and monitor vaccine efficacy against SARS-CoV-2, the virus that causes COVID-19. All currently available NAb tests are lab based and time intensive. A 10 minute cellulose pull-down test to detect NAbs against SARS-CoV-2 from human plasma is described herein. The test evaluates the ability of antibodies to disrupt ACE2 receptor—RBD complex formation. The simple, portable, and rapid testing process relies on two key technologies: (i) the vertical flow paper-based assay format and (ii) the rapid interaction of cellulose binding domain to cellulose paper. This test gives above 80% sensitivity and specificity and up to 93% accuracy as compared to two current lab-based methods using COVID-19 convalescent plasma. Importantly, this approach can be easily extended to test RBD variants or to evaluate NAbs against other pathogens.

COVID-19 is the biggest pandemic of the modern era. It affects>200 million people and, to date, has killed>4 million, worldwide. To prevent transmission of SARS-CoV-2—the virus that causes COVID-19—tight restrictions on movement and social interactions have been placed on populations across the globe. While this has had some effect on preventing the spread of the virus, they have plunged the global economy into a severe contraction. A phased relaxation of these social control measures is critical to allow business, and the world economy, to recover.

Achieving herd immunity against SARS-CoV-2, either naturally or through vaccination, is the ultimate long-term goal that will allow lifting of the widespread social control measures currently in place. Neutralizing antibodies (NAbs) are generated in response to either exposure to the virus, or to a vaccine. For effective prevention of viral infections, NAbs must be generated in sufficient quantity. Screening populations for the presence of NAbs is a critical step to evaluate herd immunity against SARS-CoV-2, and to assess the effectiveness of vaccine immunization programmes, deployed in many countries since late 2020. To facilitate rapid screening of SARS-CoV-2 NAbs, NAbs detection tests that can be performed simply, rapidly and at low cost are highly desired.

Currently, NAbs are generally detected using virus neutralization tests (VNTs). Standard VNTs require handling of live virus (conventional VNT (cVNT)) or pseudovirus (pVNT), BSL3/BSL2 facility, skilled personnel, and 2-4 days processing time, thus making them unsuitable for mass testing the immune status of a population. SARS-CoV-2 initiates the process of host cell entry, by interacting with angiotensin converting enzyme II (ACE2) receptors present on the host cell via the receptor binding domain (RBD) of the spike (S) protein. Based on this observation, a rapid (1-2 h) plate-based ELISA, surrogate SARS-CoV-2 neutralization test (sVNT) has been developed using recombinant hACE2 receptor and viral RBD proteins. NAbs are detected by their ability to bind RBD and block the formation of RBD/hACE2 complexes. Though much more rapid than the standard VNTs, the sVNT still require a laboratory setting and skilled personnel, presenting a barrier to large scale screening.

Here, a rapid cellulose pull-down viral neutralization test (cpVNT) that detects SARS-CoV-2 NAbs in plasma samples within 10 minutes and that can be used at the point of care (POC) is described. The test principle relies on the interaction between RBD and ACE2 to determine the presence of NAB. To allow the test to be compatible with cellulose matrix, RBD is fused to the cellulose binding domain (CBD), generating RBD-CBD which is used as a capture reagent. ACE2 is conjugated to biotin (BA), creating ACE2-BA which is capable of interacting with various kinds of reporting molecule via BA—streptavidin (SA) interaction. In this application, SA-horse radish peroxidase (SA-HRP) bound to ACE2-BA (ACE2-BA/SA-HRP) is used as a reporting reagent. Thus, components of the test include: (i) RBD, tagged with cellulose binding domain (CBD), (ii) ACE2 receptor, tagged with biotin (BA) and (iii) streptavidin conjugated horseradish peroxidase (SA-HRP), to detect NAbs binding to the RBD on cellulose paper. Despite the simplified and very rapid testing procedure, the cpVNT exhibits comparable performance to the lab-based tests in determining the level of NAbs in COVID-19 convalescent plasma samples with accuracy well above 80% and 90%, compared to pVNT and sVNT, respectively.

Biological fluids including non-diluted or diluted plasma or serum can be used. Briefly, samples may be mixed with capture (RBD-CBD) and reporter (ACE2-BA/SA-HPR) reagents and incubated for at least 5 minutes prior to applying it onto the cellulose test strip. A washing step can be applied to wash away non-specific molecules on the cellulose surface. 3,3′,5,5′-tetramethylbenzidine (TMB) substrate solution may then be applied to generate colorimetric signal. The enzymatic colorimetric reaction can be left developed on the test strip for ˜3 minute before colorimetric signal can be measured.

In the absence or low concentrations of NAb, RBD-CBD and ACE2-BA/SA-HRP are able to form complex effectively, therefore high colorimetric signals may be observed on the cellulose test strip. In the presence of high concentration of NAb, the antibodies may compete with ACE2-BA/SA-HRP complex to bind to RBD-CBD, thereby inhibiting colorimetric signals on the test strip.

Using this developed assay workflow and construction, the test is able produce well above 80% and 90% test accuracies as compared to the lab-based pseudovirus virus neutralization test (pVNT) and surrogate virus neutralization test (sVNT) (see Results section below for more details).

### Results

Optimization of rapid paper-based cpVNT. The assay times for the currently established lab-based and commercialized NAb tests range from 1.5 hours to 4 days. A shortening of the overall assay time and a simplified workflow are primary requirements for POC NAb tests suitable for large-scale surveillance applications. Vertical flow assays are a type of assay formats that allows reagents to flow in a top-to-bottom fashion for detection of biomolecules. Vertical flow assays offer short liquid flow paths that enable rapid and controllable flow speed for handling of liquid reagent. With an aim for POC NAb test, the vertical flow assay format was selected as a test format for this study. Cellulose is a cost-effective material that can be easily manufactured at scale. It has been demonstrated that cellulose can be used as a test matrix for vertical flow assays, whereby the assay reaction is allowed on the cellulose matrix using high affinity interaction between cellulose binding domain (CBD) and the cellulose matrix. The common construction of the assay is to fuse CBD to the capture reagent. Cellulose was adopted as the test materials to bypass the surge of high nitrocellulose demand from global ramp up of rapid COVID-19 tests which presses massive risk onto the supply chain shortage. The test principle relies on a complex formation between RBD/ACE2 receptor whereby presence of NAb interferes with the RBD/ACE2 receptor complex formation, thereby reducing the reporting signal intensity. To enable the test to be compatible to cellulose paper, CBD was tagged to RBD (RBD-CBD), allowing the RBD protein to be captured rapidly and at high affinity onto cellulose surface. ACE2 receptor was engineered to be a reporting molecule. This was done by tagging biotin (BA) on to ACE2, creating ACE2-BA. Horse radish peroxidase (HRP) conjugated streptavidin (SA), SA-HRP, was used as a colorimetric signal generator. A complex of ACE2-BA/SA-HRP was used to generate colorimetric signal via application of 3,3′,5,5′-tetramethylbenzidine (TMB)/H₂O₂ which hydrolyzes HRP, producing vivid blue color signals.

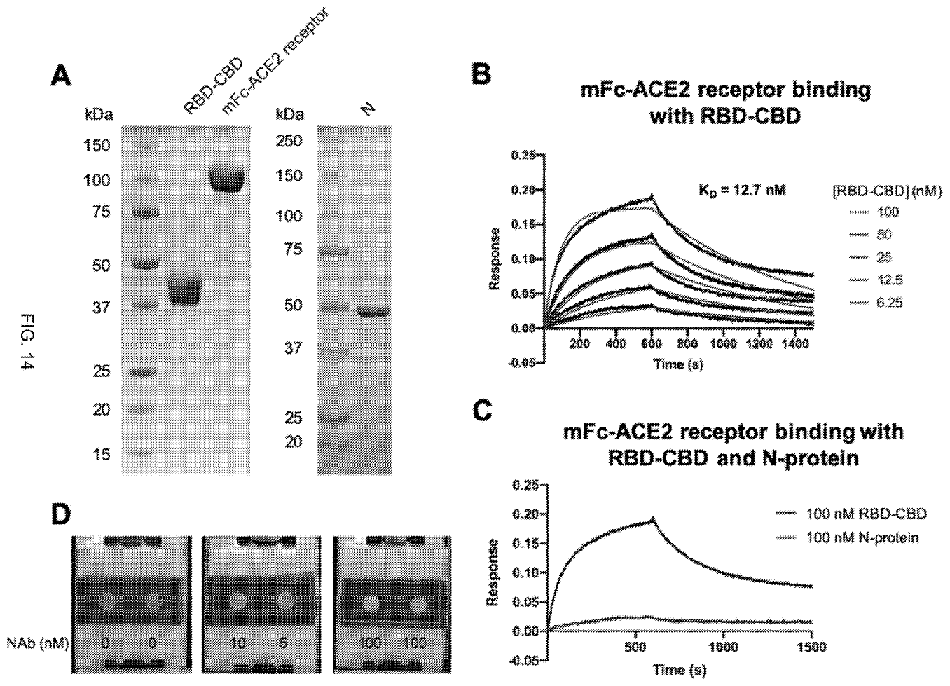

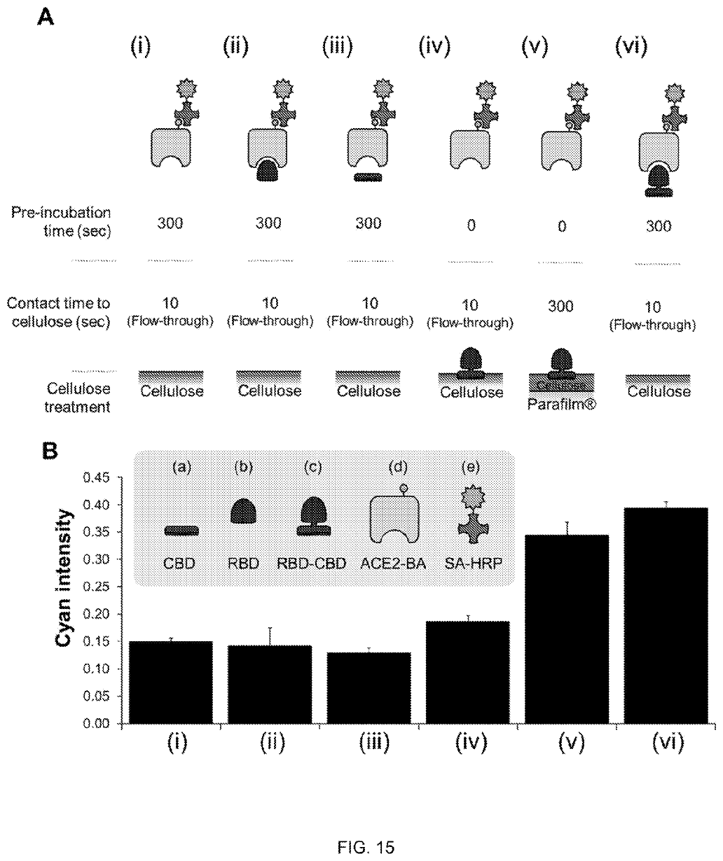

To construct the test, recombinant (i) RBD-CBD and (ii) biotin (BA) tagged monoFc-ACE2 receptor proteins were expressed, purified (FIG. 14A) and evaluated for their kinetic properties using bio-layer interferometry (BLI). Various concentrations of RBD-CBD ranging from 6.25 nM-100 nM were used. Data showed specific binding between monoFc-ACE2 and RBD-CBD event at a low concentration of RBD-CBD at 6.25 nM (FIG. 15B). The pair demonstrated high affinity toward each other with a K_{D} of 12.7 nM (FIG. 14B) which is comparable to previously reported K_{D} ranging from 4.7-15.2 nM. In contrast, SARS-CoV-2 structural, nucleocapsid (N) protein was tested on ACE2-BA to assess specificity of the protein. BLI data showed minimal interaction of BA-ACE2 with N protein even at 100 nM of N (FIG. 14C), indicating that recombinant BA-ACE2 is highly specific to RBD protein.

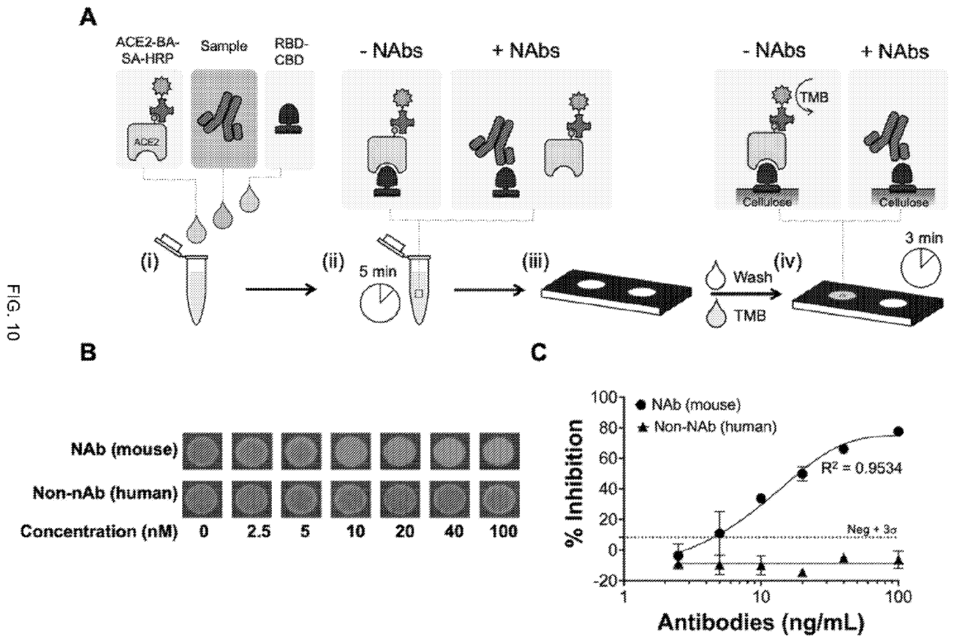

To engineer the vertical flow assay (FIG. 9), wax ink was printed on to cellulose paper to create hydrophobic boundary and define liquid flow path. Liquid flow rate was controlled by stacking 3 layers of cellulose paper together (FIGS. 9A and D) whereby the top layer has a smaller, 5 mm diameter, of hydrophilic area without wax (testing region) and the lower two layers have larger hydrophilic areas of 6 mm diameters. One unit of cellulose test strip comprises two testing regions for testing of two reactions (FIGS. 9A and B). The distance between the center of the two testing spots is 12.5 mm. (FIGS. 9A, B and D). A folded Kim Wipes paper was used as the absorbent pad (FIGS. 9A and E). An acrylic sheet with 2 mm thickness was cut using a laser cutter (Epilog Fusion Edge Laser System, USA) into two pieces of an acrylic manifold, each with dimension of 30×50 mm² (FIGS. 9A, B, C and F). One of the acrylic pieces contains an opening of 25×10 mm² (FIGS. 9A and C). The other piece of acrylic was equipped with two pieces of 3 mm thickness acrylic sheets which were used to form spacers for clamping of cellulose test unit (FIGS. 9A, B and F). Three layered cellulose papers were stacked on top of the folded Kim Wipes and secured together using the acrylic manifold and paper binders (FIGS. 9A and B). The 3 mm spacers between the two pieces of the manifold provide consistent pressure between different cellulose test units prepared. The test strip units provided a consistent liquid flow speed of ˜10 sec when 40 μL of liquid were used.

To capture the colorimetric signal from the cellulose vertical flow assay, Xiaomi Redmi A9 phone was used to capture the image and save the image in a .jpg format. To fix the camera distance and angle as well as to prevent interference from the surrounding light, a ‘light box’ was created. The box has a W×L×H dimension of 150×230×90 mm (FIG. 9G). A void was made at the top face of the box to provide an access to the phone camera (FIG. 9H). Distance between cellulose test unit to the camera was 85 mm. Fixtures were made to fix locations of the test strip unit and the phone whereby the test strip unit fixture was secured to the base of the light box (FIG. 9I) and the camera fixture was secured to the top face of the light box (FIGS. 9G and H). The internal faces of the box were equipped with warm-white LED light strips (FIG. 9I). To prevent shadows on the cellulose test unit, white, cylindrical shaped papers were constructed to cover the LED strip, thus diffusing light from the LED strips. Example of images captured from the phone camera and the light box are shown in FIG. 14D. Images were analyzed using the open-source ImageJ software from National Institute of Health (NIH), USA. Only the circular areas of the hydrophilic testing regions were used for analysis. To optimize the image analysis, images were separated into different channels in RGB and CYMK color spaces. Comparing gradient color of the blue signals from HRP/TMB generated on different cellulose papers, signals recorded from cyan channel provided the highest signal (RBD-CBD+ACE2-BA/SA-HRP+TMB) over noise (ACE2-BA/SA-HRP+TMB) ratio as compared to other channels in the RGB and CYMK color spaces. Therefore, the intensity of the cyan channel in the CYMK color space was used for image analysis.

Different cellulose vertical flow assay formats were performed to determine the most effective assay workflows (FIG. 15). Results showed that application of the reporting complex (ACE2-BA/SA-HRP) onto the pre-immobilized RBD-CBD (group (iv) in FIG. 15) generated only a slight change in cyan signal intensity as compared to control groups (group (i)-(iii) in FIG. 15). It was speculated that the rapid 10 sec. liquid flow speed contributed significantly to the inefficient capture of the reporting complex onto the RBD-CBD and cellulose paper. Significant signal improvement was observed when the reaction was entrapped on the paper for 5 min (group (v) in FIG. 15). Retaining the assay reaction onto the cellulose surface for minutes would complicate the assay workflow and not ideal for POC settings. CBD is known to interact rapidly and effectively to cellulose matrix. Based on this knowledge, the assay was optimized by mixing all reagents in one ‘pre-mix’ solution and incubated for 5 minutes prior to applying the reaction to the cellulose test unit. With this design, the cellulose test unit produced highest cyan signal intensity as compared to other designs (group (vi) in FIG. 15). Based on this observation, the ‘pre-mix’ format is selected for the rapid NAb test and referred to as cellulose pull-down VTN (cpVNT) following the assay principle of the optimized test format. In addition, protein kinetic data from BLI experiment showed that >80% of RBD/ACE2 receptor complex can be formed within 5 min (FIG. 14B), ensuring that most of the RBD/ACE2 complexes were formed with in the incubating period.

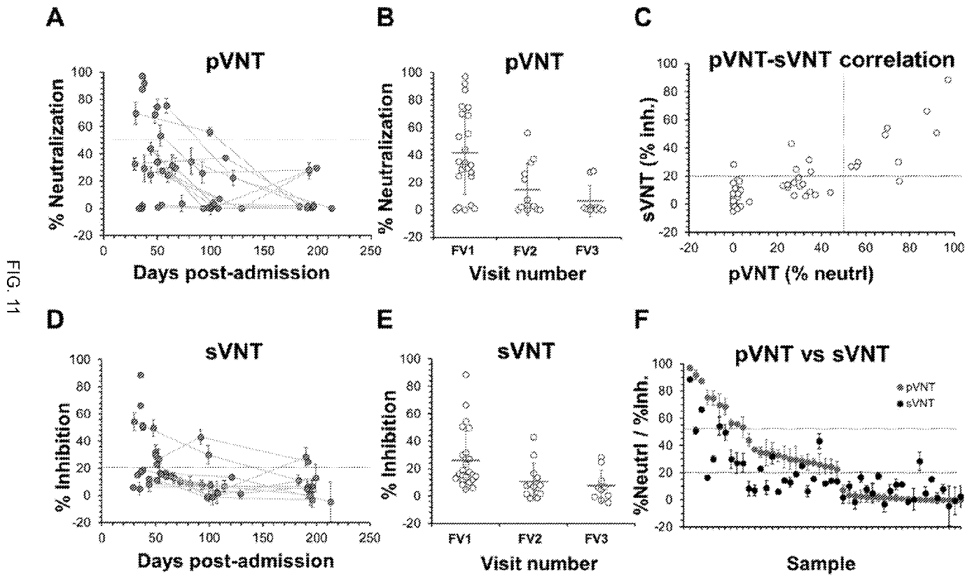

Based on these optimizations, the assay can be performed by mixing the plasma sample with the reagents and incubating for 5 min to allow efficient formation of RBD-CBD/NAbs or RBD-CBD/ACE2 complexes in aqueous phase before applying it onto the cellulose paper (FIG. 10A). The high affinity vertical flow assay format ensures that most of the RBD-CBD complexes formed are exposed to the paper matrix and effectively captured through the rapid and high affinity interaction of CBD and cellulose. The high affinity of CBD to cellulose ensures a minimal loss of the complex during the washing step.

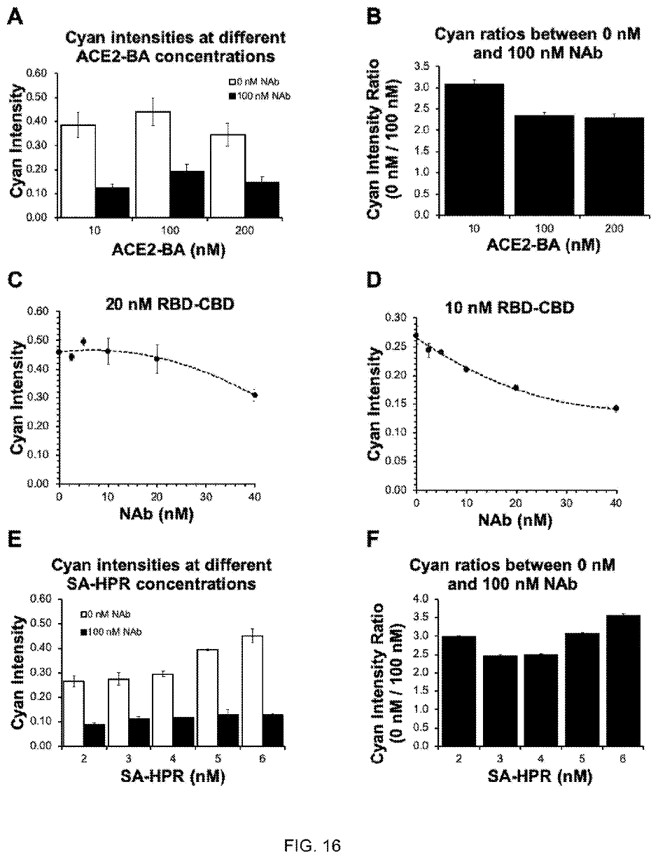

Concentrations of RBD-CBD and ACE2-BA/SA-HRP were further optimized to obtain the highest signal difference between presence and absence of NAb (FIG. 16). To do so, human serum containing 0 or 100 nM NAb were used for signal comparison. Ratio between maximal and minimal cyan intensities obtained from 0 and 100 nM NAb, respectively, were used to calculate the signal ratio. Concentration of ACE2-BA was first optimized using fixed concentrations of 20 nM and 2 nM of RBD-CBD and SA-HRP, respectively. Signals were captured at 3 min following addition of TMB and washing solution. Results showed that 10 nM ACE2-BA produced highest cyan intensity ratio from 0 nM:100 nM NAb (FIGS. 16A and B). Therefore, 10 nM ACE2-BA was selected for subsequent experiments. To optimize for RBD-CBD concentration, ACE2-BA and SA-HRP concentrations were fixed at 10 nM and 2 nM, respectively. Two different concentrations of RBD-CBD at 10 and 20 nM were tested. Results showed that both concentrations produced similar cyan intensity ratio from 0 nM:100 nM NAb which were at ˜2.1. Therefore, different concentrations of NAb were tested to further explore the different cyan intensity signals at various NAb concentrations. This showed that RBD-CBD at 10 nM produced clearer distinguishable cyan intensity signals at lower NAb concentrations (FIGS. 16C and D) and this concentration was selected for further studies. For optimization of SA-HRP, RBD-CBD and ACE2-BA concentrations were fixed at their optimized concentrations of 10 nM. Various concentrations of SA-HRP were tested in serum containing 0 nM or 100 nM of NAb. Results showed that 6 nM SA-HRP produced highest ratio of cyan intensity from 0 nM: 100 nM NAb (FIGS. 16E and F). Therefore, 6 nM SA-HRP was selected for subsequent analysis. Altogether, optimized concentrations of RBD-CBD, ACE2-BA and SA-HRP for cpVNT were 10 nM, 10 nM and 6 nM, respectively.

The rapid paper-based cpVNT performance. To validate the cpVNT test, different concentrations of mouse anti hSARS-CoV-2 neutralizing antibodies were spiked into non-diluted human plasma and evaluated for their ability to inhibit RBD-CBD/ACE2 complex formation (FIG. 10A). This approach demonstrated efficient inhibition of RBD-CBD/ACE2 complex formation for a dynamic range of 10-100 nM SARS-CoV-2 NAbs (FIGS. 10B and C). The cpVNT was tested using plasma samples containing non-neutralizing human anti-RBD antibodies produced from the lab. Results showed that only minimal inhibitory signals were observed from these samples even at high concentrations of the non-NAbs (FIGS. 10B and C). These data indicate that the test is highly specific to the SARS-CoV-2 NAbs but not to non-NAbs. The data shows a strong relationship between antibody concentrations and inhibition of complex formation (FIG. 10C) with a limit of detection (LOD), calculated by using ‘mean negative+3SD’ formula, of 10 nM NAbs.

Assessments of SARS-CoV-2 immunological profile from COVID-19 convalescent plasma. Prior to validating the cpVNT using clinical samples, general assessments of the clinical samples were performed. Plasma samples from 24 confirmed COVID-19 patients were collected between 29 days and 73 days (median of 49 days) post positive PCR test (follow up visit 1 (FV1)). Subgroups of patient plasma samples were collected on two subsequent occasions (FV2 (n=13); and FV3 (n=10)) (Table 2).

TABLE 2

| | | No. days post admission | | | No. of days | |
|---|---|---|---|---|---|---|
| | | FV1 | | | of symptoms | |
| | | (Follow | | | before | |
| Sample | Severity | up visit) | FV2 | FV3 | admission | Remark |
| P01 | Mild | 38 | | | 7 | |
| P02 | Moderate | 50 | | | 6 | |
| P04 | Mild | 29 | 114 | 196 | 14 | |
| P06 | Mild | 30 | 99 | 183 | 8 | |
| P07 | Severe | 36 | | | 5 | |
| P08 | Severe | 36 | | | 7 | |
| P09 | Mild | 36 | | | 3 | |
| P10 | Mild | 35 | | | 4 | |
| P11 | Mild | 33 | | | NA | Asymptomatic |
| P13 | Mild | 51 | 82 | 194 | −9 | Symptom started |
| | | | | | | on Day 9 post |
| | | | | | | admission |
| P14 | Mild | 48 | | | NA | Asymptomatic |
| P15 | Mild | 38 | 129 | 192 | 12 | |
| P19 | Mild | 53 | 92 | 192 | −3 | Symptom started |
| | | | | | | on Day 3 post |
| | | | | | | admission |
| P20 | Mild | 44 | 93 | 191 | 0 | |
| P21 | Severe | 59 | 121 | 213 | 0 | |
| P22 | Mild | 54 | 100 | 199 | 0 | |
| P23 | Mild | 67 | 107 | 190 | −7 | Symptom started |
| | | | | | | on Day 7 post |
| | | | | | | admission |
| P24 | Mild | 73 | | | 0 | |
| P25 | Severe | 44 | | | 0 | |
| P30 | Mild | 51 | 98 | | 30 | |
| P37 | Mild | 60 | 101 | | 0 | |
| P42 | Mild | 64 | 103 | 196 | NA | Asymptomatic |
| P45 | Mild | 50 | | | 7 | |
| P48 | Mild | 55 | 108 | | −8 | Symptom started |
| | | | | | | on Day 8 post |
| | | | | | | admission |

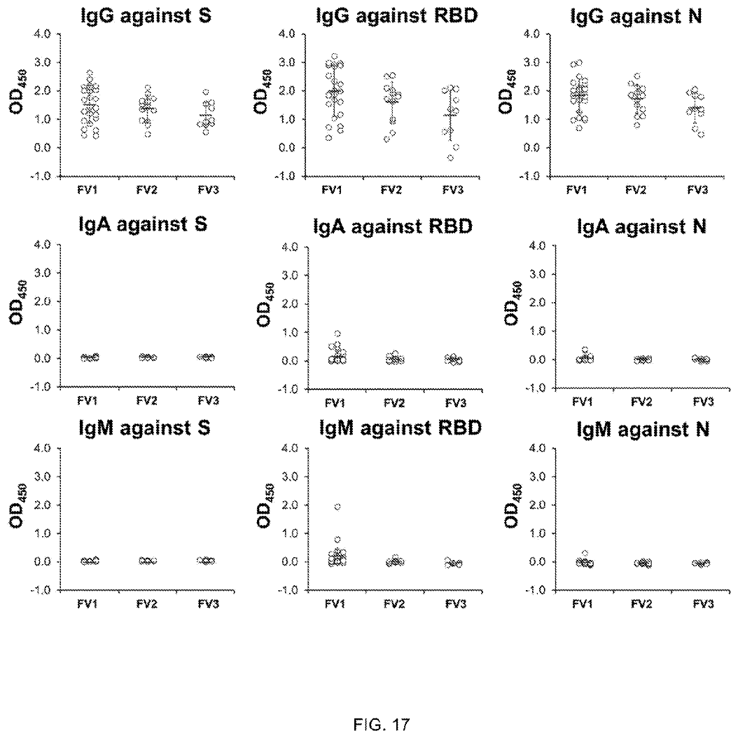

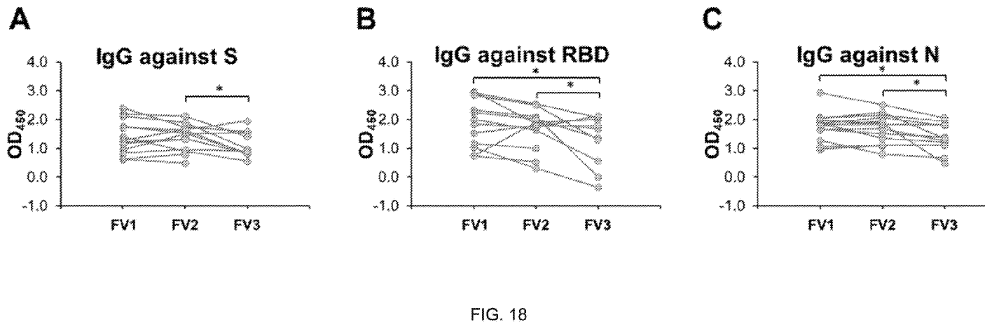

General assessments were performed, for each convalescent plasma sample, to obtain immunological profiles against SARS-CoV-2 and to determine reference points for cpVNT evaluation. Antibody subtypes IgA, IgM and IgG against SARS-CoV-2's S, RBD, and nucleocapsid (N) proteins were evaluated using ELISA (FIG. 17). IgA and IgM levels against RBD peaked in FV1 in several samples and decline to baseline in FV2 and FV3. IgA and IgM against S and N were found to be low throughout the enrolment period. The minimal levels of IgA and IgM observed in this study are in good agreements with recent studies showing that IgA and IgM against RBD and N peaked at 14-20 days post symptom onset and started to wane after ˜20 days. Plasma samples collected for this study began on day 29 post admission in which IgA and IgM are expected to be low or declining. IgG levels were found to be higher than IgA and IgM (FIG. 17). No statistical difference in IgG levels against all three SARS-CoV-2 biomarkers were observed from FV1 and FV2 (FIG. 18). Significant reduction of 15%-25% in IgG levels against all biomarkers were seen in FV3 as compared to FV2 (FIG. 18). These findings suggest that IgG levels against S, RBD and N proteins were maintained at high level for around 4 months post admission and started to decline slowly after 5-6 month.

To determine the levels of NAbs present in different plasma samples, two lab-based VNTs were used (i) chemiluminescent-based pseudovirus VNT (pVNT) and (ii) a modified format of the published ELISA-based surrogate VNT (sVNT) (Tan, C. W. et al. A SARS-CoV-2 surrogate virus neutralization test based on antibody-mediated blockage of ACE2-spike protein-protein interaction. Nat. Biotechnol. 38, 1073-1078 (2020)). The established pVNT protocols (Jahrsdörfer, B. et al. Characterization of the SARS-CoV-2 Neutralization Potential of COVID-19-Convalescent Donors. J. Immunol. 206, 2614-2622 (2021) and Nie, J. et al. Quantification of SARS-CoV-2 neutralizing antibody by a pseudotyped virus-based assay. Nat. Protoc. 15, 3699-3715 (2020)) were used as references for pVNT performed in this study. pVNT determined NAb status by measuring chemiluminescent signals from cells infected with pseudovirus. Presence of NAbs prevent virus from infecting the cells thereby reducing chemiluminescent signals. Based on the reference studies, a wide range of sample dilution factors from 1-10⁶ were used to determine the effective cut-off between presence and absence of NAb. A 50% signal inhibition was indicated as an effective value to distinguish between presence and absence of NAb. During the test optimization, it was found that, a dilution factor of at least 1:80 is necessary to minimize false positives produced by healthy control samples, therefore a fix 1:80 dilution factor is used in this study. The chemiluminescent signals measured were normalized with the signals from non-infected and pseudovirus infected cells. The status of NAbs was expressed as neutralization percentage.

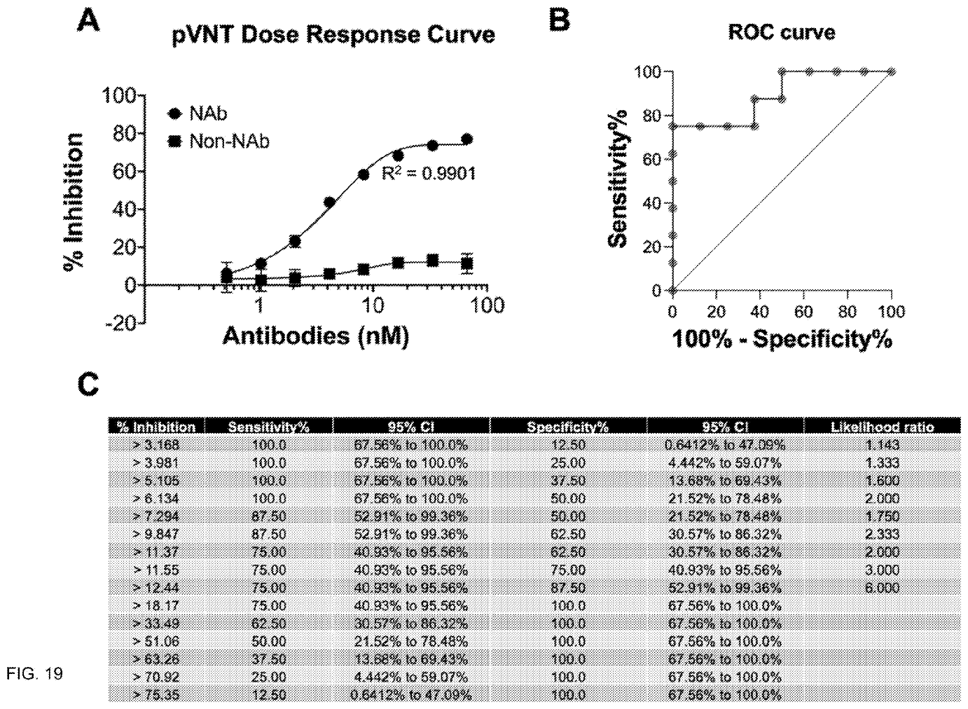

For sVNT, while it retains the same test principles as the published method, the modified sVNT configuration used recombinant RBD protein as a capture reagent and ACE2 receptor conjugated with HRP as a reporter reagent. In this test format, the presence of NAbs causes signal reduction that can be expressed as inhibitory percentages. The higher percentages represent higher level of NAbs and vice versa. 10×dilution were chosen as the lowest dilution factors that give reliable results without showing false positives from healthy control samples. The cut-off value for sVNT was determined by analyzing the test sensitivity and specificity using known concentrations of NAbs and non-NAbs spiked in plasma samples (FIG. 19). Receive Operating Characteristic (ROC) curve showed that the test maintained a high sensitivity of 75% while achieving a high specificity of 100% at the inhibitory percentage of ˜20%, therefore 20% inhibition is used as a cut-off value to distinguish positive from negative NAb status. Using this cut-off, the sVNT is sensitive to detect RBD-specific neutralizing antibodies at a concentration of 313 ng/mL (2 nM), much lower than the average concentration of these antibodies in COVID-19 convalescent patients.

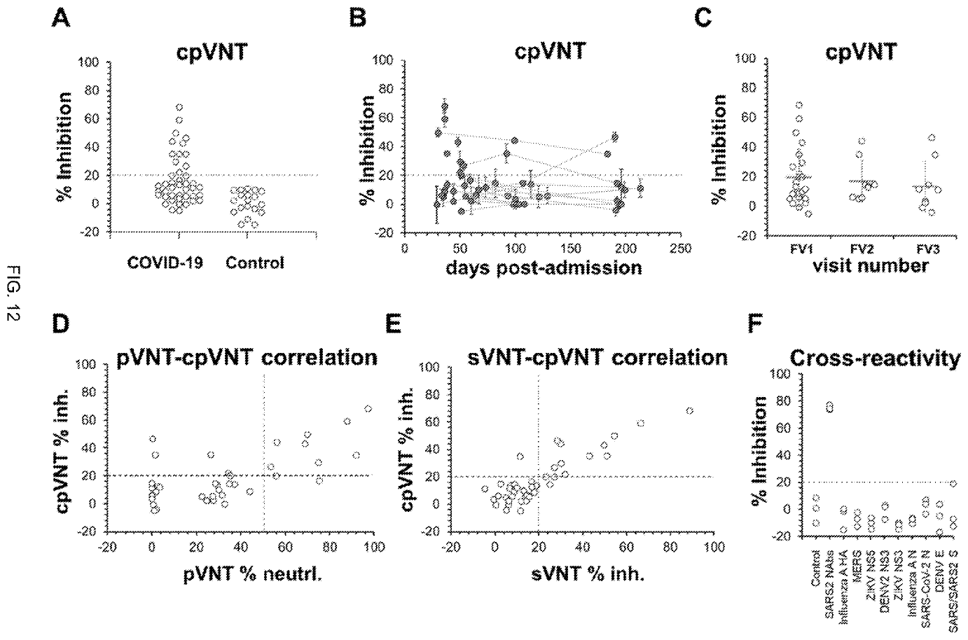

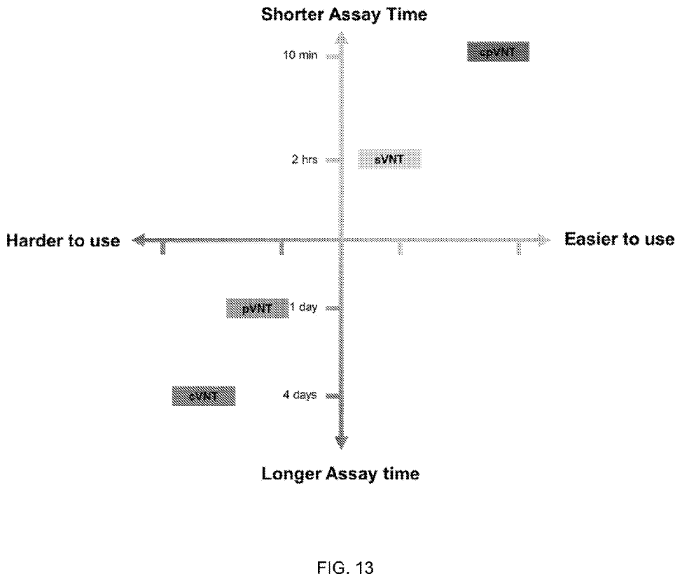

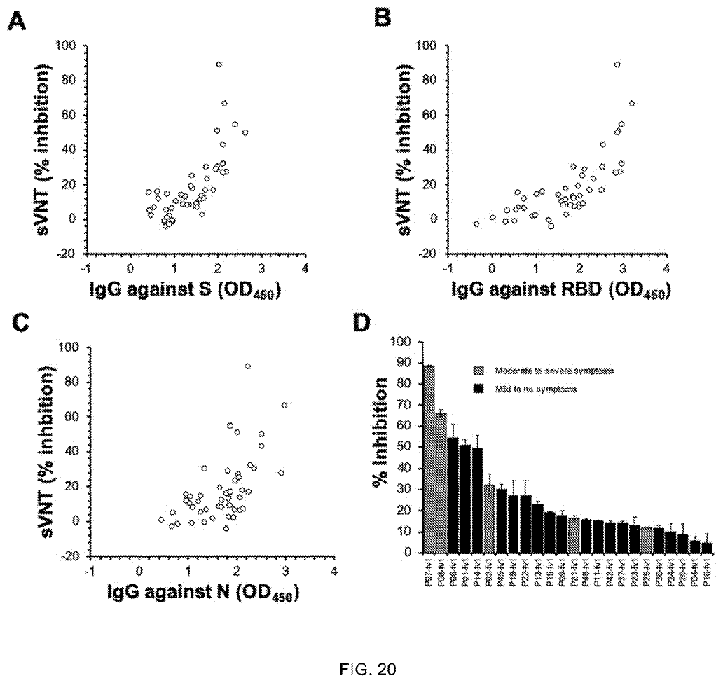

Data obtained from pVNT and sVNT are shown in FIGS. 11A, B, D and E. The pVNT and sVNT exhibit a high Pearson correlation coefficient of 0.8 (FIGS. 11C and F), indicating that both tests produced results that are in good concordance. In addition, it was observed that NAb status is highly correlated to IgG levels against S and RBD with Pearson coefficients of 0.72 and 0.74, respectively (FIGS. 20A and B) and moderately correlated with IgG level against N with a Pearson coefficient of 0.59 (FIG. 20C). No clear correlation is observed between NAb status and disease severity in the study (FIG. 20D), in line with the recent observation reported by a study.

pVNT presents a test format that is closely related to events that occur in a physiological condition. Therefore, pVNT will be used as a baseline to determine the accuracy of sVNT. Using the defined 50% cut-off for pVNT and 20% cut-off for sVNT, the sVNT provides test sensitivity and specificity of 90.0% and 86.5%, respectively with an overall accuracy of 87.2% (with 95% CI of 74.3% to 95.2%) (Table 3).

TABLE 3

| Statistic | Value | 95% CI |
|---|---|---|
| Sensitivity | 90.0% | 55.5% to 99.8% |
| Specificity | 86.5% | 71.2% to 95.5% |
| Positive Predictive Value* | 64.3% | 43.7% to 80.7% |
| Negative Predictive Value* | 97.0% | 83.2% to 99.5% |
| Accuracy* | 87.2% | 74.3% to 95.2% |

It is also observed that most samples showed negative NAb status (FIG. 11). Majority of the positive NAbs detected came from FV1 samples in both test formats.

Evaluating of cpVNT performance using COVID-19 convalescent plasma. To evaluate the ability of the cpVNT in determining the level of NAbs, results obtaining from cpVNT using COVID-19 convalescent plasma, from different visits, were plotted against the pre-COVID plasma samples collected from earlier studies. Based on these data, the inhibitory signal cut-off which distinguished positive from negative NAbs levels was determined at 20% (FIG. 12A). Negative inhibitory percentages were observed from some data points. The negative values were due to the higher cyan intensity signals as compared to the reference point calculated from mean value of pre-COVID and non-infected samples. In the context of cpVNT, it can be interpreted that no NAbs were detected from these samples. In a similar fashion to pVNT and sVNT (FIG. 11), results from cpVNT showed that most of the COVID-19 convalescent samples exhibit negative NAb status with positive status observed mostly from FV1 (FIGS. 12B and C). An average value of NAbs from FV1 falls between the designated cut-off value and average values from FV2 and 3 fall below the cut-off value. Similar trends were also observed from pVNT and sVNT (FIGS. 11B and E).

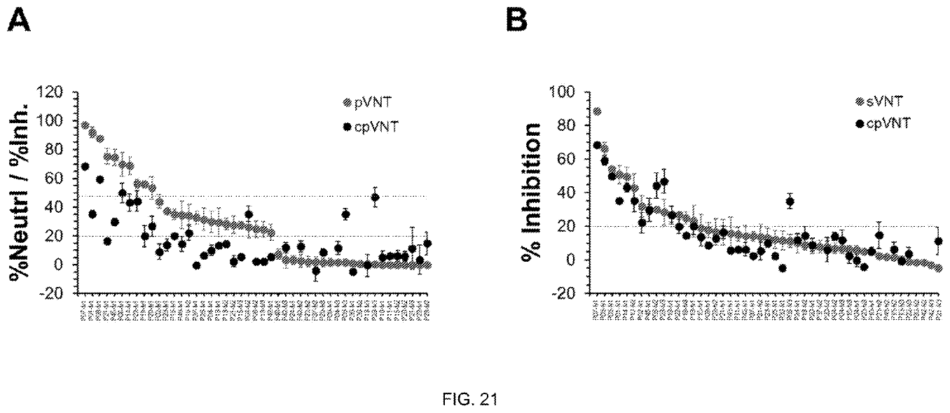

Data obtained from cpVNT show a high correlation with pVNT and sVNT with Pearson correlation coefficients of 0.70 and 0.87, respectively, (FIGS. 12D and E and FIGS. 21A and B). As compared to pVNT, cpVNT exhibits the test sensitivity and specificity of 80.0% and 84.4%, respectively with an overall test accuracy of 83.3% (with 95% CI of 68.6% to 93.0%, Table 4).

TABLE 4

| Statistic | Value | 95% CI |
|---|---|---|
| Sensitivity | 80.0% | 44.4% to 97.5% |
| Specificity | 84.4% | 67.2% to 94.7% |
| Positive Predictive Value* | 61.5% | 40.31% to 79.1% |
| Negative Predictive Value* | 93.1% | 79.5% to 97.9% |
| Accuracy* | 83.3% | 68.6% to 93.0% |

As compared to sVNT, cpVNT exhibits a sensitivity and specificity of 85.71% and 96.55%, respectively, with an overall test accuracy of 93.02% (with 95% CI of 74.37% to 96.02%, Table 5).

TABLE 5

| Statistic | Value | 95% CI |
|---|---|---|
| Sensitivity | 85.7% | 57.2% to 98.2% |
| Specificity | 96.6% | 82.2% to 99.9% |
| Positive Predictive Value* | 92.3% | 63.4% to 98.8% |
| Negative Predictive Value* | 93.3% | 79.5% to 98.1% |
| Accuracy* | 93.0% | 80.9% to 98.5% |

*These values depend on the prevalence of the disease. The prevalence is calculated from the sample size. It may not reflect the real disease prevalence.

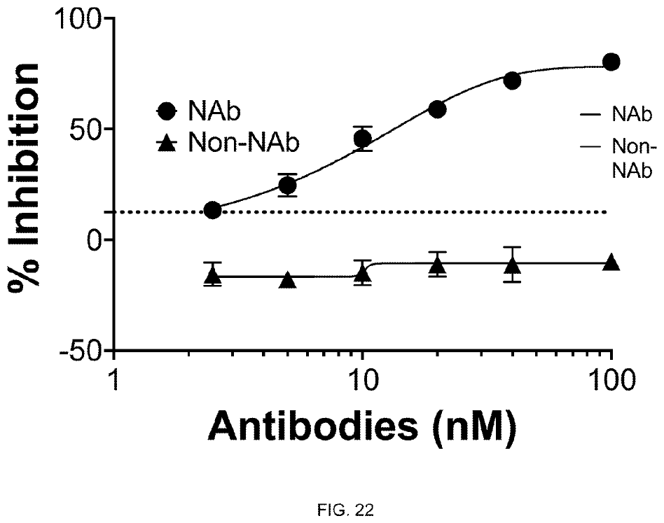

Cross reactivity tests were performed to ensure the test specificity. High concentrations (100 nM) of IgG against different viruses were spiked in plasma from healthy controls and tested on cpVNT. Data show minimal cross reactivity to antibodies against other viruses, or to SARS-CoV-2 S and N proteins (FIG. 12F), which possess non-neutralizing ability, suggesting that cpVNT is highly specific to SARS-CoV-2 NAbs. cpVNT specificity is comparable to the commercialized lab-based sVNT. In addition to plasma, the cpVNT was also tested using human serum samples. Results showed dose response curves that are comparable to the plasma samples (FIG. 22 and FIG. 10C) with an improved LOD of 5 nM, compared to 10 nM in plasma.

Altogether, the cpVNT offers a rapid test, that can be performed in ten minutes, for reliable detection of NAbs against SARS-CoV-2. Accuracy of cpVNT, compared to the lab-based methods are well above 80% and 90%, for pVNT and sVNT. The test can be performed both in plasma and serum samples without dilution, therefore facilitating simple workflow at POC settings.

Unlike other report which predicted that high level of NAb could be detected from convalescent samples, most of the convalescent samples collected for this study showed relatively low NAb level from all three VNT formats tested. The plasma samples collected for this study begun at ˜1 month post admission. Samples containing NAb came mostly from FV1. pVNT, sVNT and cpVNT detected 10, 9 and 9 NAb positive samples, which accounted for 41.7%, 37.5% and 47.4%, of the sample size, respectively. pVNT showed 1 and 0 positive samples in FV2 and 3, whereas sVNT and cpVNT, each, showed 2 positive NAbs samples in FV2 and 2 in FV3. Only 3 patients which showed positive NAb status in FV1 completed all 3 visits. Although a trend of reduction in NAbs is observed in these 3 samples, the sample sizes are too small to observe statistical differences in all test formats. Aligning with the findings, a study reported that a substantial number of convalescent samples did not produce sufficient NAb to be detected using sVNT. In addition, a large subgroup of convalescent population showed rapid waning of NAb at 2-month post symptom onset, a timeline in which majority of samples were collected for this study.

### Discussion

Global efforts are underway to improve SARS-CoV-2 surveillance and manage long term prevention of COVID-19. Assessment of SARS-CoV-2 NAbs is one of the key surveillance criteria required to evaluate herd immunity and the impact of SARS-CoV-2 on a larger population scale. Existing technologies for NAb detection (cVNT, pVNT and sVNT) all require laboratory facilities, skilled personnel and long execution times (1 hr-4 days) that are not favorable for large-scale surveillance outside a laboratory setting. cpVNT provides a robust NAb surveillance detection test, that is simple, rapid and can be easily conducted both inside and outside of laboratory settings in as little as 10 minutes. In addition to the cpVNT performance evaluated in this study, it is now feasible to compare its performance to the commercialized sVNT, c-Pass™ from GenScript®.

SARS-CoV-2 vaccination programmes have been rolled out in many countries, since late 2020, initially providing vaccines to healthcare frontline workers and members of high-risk communities. A serological neutralizing antibody test is a valuable companion test to evaluate the effectiveness of available vaccines. Data from different VNT formats obtained from this study suggested that ˜37-48% of the samples showed positive NAb status in the first 1-2 months (FV1), post admission. The number declined to ˜7-15% in the subsequent visits. However, due to the small sample size of follow up visits, no statistical difference was observed from different visits. Nonetheless, findings from other studies demonstrated that NAbs declined gradually over 3-month period post symptom onsets, therefore suggesting that vaccine boosters may be required to maintain the immunity status at a desirable level. The rapid neutralization test described here would be a suitable tool to regularly assess the immune status of individuals, particularly in the vulnerable population. The simple nature and speed of this test provides an accessible POC tool, which can be used at community clinics or in low resource settings, to prioritize vaccine administration. In addition, the test can be rapidly adapted to evaluate the efficiency of NAbs to new virus variants and thereby guide the decision-making process in relation to the need of new booster vaccines.

Despite a number of lateral flow assay (LFA) tests available for detection of antibodies against SARS-CoV-2, to knowledge, only one pre-print report is found for rapid NAbs test. In most LFA antibody detection tests (rapid serology tests), specific antigens are either immobilized on the testing matrix or used as reporting molecules whereas the counter reporting/capturing part are anti-IgA/IgM/IgG antibodies. It was reported that, when RBD and anti-IgA/IgM/IgG antibodies are used for detection of NAbs in the plate-based ELISA format, non-NAbs are often detected along with NAbs (due to antibodies that bind to RBD but do not possess neutralizing ability). This method is thus unable to predict the level of NAbs accurately. Adapting a NAbs test to a LFA format seems feasible due to the well-established LFA technology. However, with the test format employed for the cpVNT and sVNT reported in this study, it would be challenging for LFA to report a loss of a colorimetric signal as a positive result, particularly when anti-IgA/IgM/IgG were to use as reporting molecules. ˜15-20 incubation time is required for LFA, thus allowing substantial amount of time for non-specific binding to occur at the test line on the LFA test strip. To overcome this issue, a suitable control system would have to be designed to ensure that a positive colorimetric signal generated (i.e., lack or low level of NAb) is not due to non-specific binding.

The rapid cpVNT neutralization test developed in this study identifies and measures the very specific interaction between RBD and ACE2 receptor. Non-NAbs will not interfere with the RBD/ACE2 receptor complex formation and the signal detected is specific to neutralizing antibodies (FIGS. 10B-C and 12F). In addition, unlike the LFA format that requires 15-20 min incubation time, the cpVNT developed in this study requires only 5 min interaction time between NAb/RBD or ACE2/RBD, thus providing minimal time for non-specific interactions and only high affinity interactions were anticipated to be captured on the cellulose surface.

The sudden high demand for LFA COVID-19 rapid diagnostic tests has created a worldwide shortage of materials required for LFAs, particularly the nitrocellulose membrane, leading to supply chain issues. The cpVNT presented in this study utilizes cellulose membrane which is more economical to produce and supply chains are unimpeded. In addition, cellulose paper can be easily manufactured, enabling large-scale production of the test strips in a very short period, thereby facilitating mass manufacture of the test with low production cost.

Plasma/serum is currently optimized for cpVNT. As such, a device capable of separating plasma from whole blood is needed for the POCT applications. Different aspects of test stability are currently being investigated. Based on the preliminary data, with the right preservatives and additives the cellulose test paper can last up to 6 months when kept at ambient temperature (25° C.) without controlling of humidity. The test papers last up to 3 years in a desiccator. RBD-CBD and ACE2-BA retain their activities for at least 3 months when kept in optimized conditions. In the POC settings, a test strip may comprise one ‘Test’ spot and one ‘Control’ spot, whereby the control spot hosts a chemical reaction that indicates active function of the reagents. It is critical for the test to have a control spot because the positive result is derived from loss of colorimetric signal. The control spot ensures that the loss of signal at the test is due to binding of NAb to RBD-CBD and not the malfunction of the chemistry reaction. The optimization of the control spot was started and it was found that immobilizing of RBD-CBD at high concentrations on the cellulose paper could potentially serve as a control spot reaction to capture ACE2 tagged to reporting molecules from the assay mixture. The preliminary data showed that this design of control spot produced high cyan intensity signals regardless of presence or absence of NAb in the samples. For signal analysis of cpVNT, a pre-set cyan intensity value could be pre-determined from pre-COVID or non-infected samples during the assay optimization stage. This value can be implemented for the POC applications in which the pre-determined cyan intensity value defines a reference signal for analysis of the cpVNT test results.

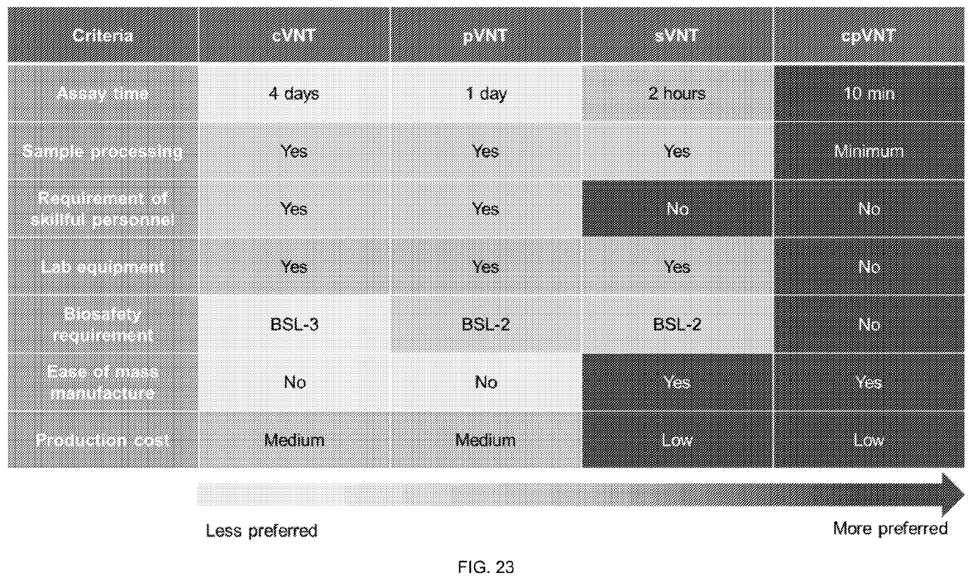

In conclusion, a rapid, paper-based cpVNT that can be used at POC for effective identification of neutralizing antibodies against COVID19 is developed. Comparison of cpVNT against the existing VNTs, including, the pVNT and the sVNT (FIG. 12 and FIG. 23) shows that cpVNT offers a highly competitive solution as compared to existing technologies, including the very rapid execution time (10 min) and ease of operation (no need for laboratory facilities and does not require skilled operators). This represents a significant advance in tackling the pandemic and has far reaching applications.

### Materials and Methods

Materials. Materials were purchased from the following sources, mouse anti-SARS-CoV-2 NAb (cat #40591-MM43-100), monoclonal mouse anti Influenza A H10 Hemagglutinin/HA NAb (cat #40359-M001), monoclonal mouse anti Influenza A Nucleoprotein IgG (cat #11675-MMO3T) and polyclonal rabbit anti SARS-CoV-2 nucleocapsid protein IgG (cat #40588-T62) from Sino Biological, USA; monoclonal rabbit anti MERS Coronavirus Spike protein NAb (cat #MA5-29975) and polyclonal rabbit anti Dengue Virus Type 2 NS3 protein IgG (cat #PA5-32199) from Invitrogen, USA; polyclonal rabbit anti Zika virus NS5 protein IgG (cat #GTX133312), polyclonal rabbit anti Zika virus NS3 protein IgG (cat #GTX133309), monoclonal mouse anti Dengue virus envelope protein IgG (cat #GTX629117) and monoclonal mouse anti SARS-CoV-2 spike protein IgG (cat #GTX632604) from GeneTex, USA. Other chemicals were of analytical grades from Merck, Singapore, otherwise stated.

Collection of clinical samples. Collection of COVID-19 convalescent samples were reviewed and approved under the DSRB reference #2020/00120, National University of Singapore (NUS). Peripheral blood was collected in EDTA blood tubes and subsequently diluted with an equal amount of sterile PBS. This was then gently layered on top of 13 mL Ficoll-Plaque density gradient media (GE Healthcare) in a 50 mL Falcon tube. The tube was centrifuged at 2400 rpm for 30 min with acceleration and deceleration set at 0. Plasma was harvested from the top layer and stored at −80° C. Buffy coat layer was washed with sterile PBS at 2000 rpm for 6 min followed by another wash at 1500 rpm for 5 min. Peripheral blood mononuclear cells (PBMNC) were harvested, resuspended in freezing media containing 90% FBS (Hyclone)+10% dimethyl sulfoxide (Sigma Aldrich), and stored in liquid nitrogen.

Collection of pre-COVID samples were reviewed and approved by Institutional Review Board of Nanyang Technological University, Singapore (IRB 003/2010, IRB 11/08/03, IRB 13/09/01, IRB-2016-01-045 and IRB-2020-11-047). The whole blood was donated by healthy adult volunteers at the National University Hospital, Singapore. Informed consents were obtained from all donors in accordance with the approved protocols. Whole blood samples collected were centrifuged at 2000 rpm for 10 min. Plasma was collected and stored at −80° C. until used.

Isolation and cloning of SARS-CoV-2 Spike RBD-specific human antibodies. Memory B cells were isolated from PBMNC derived from blood samples drawn from COVID-19 convalescent patients using a Human Memory B cell isolation kit (Miltenyi Biotec, #130-093-546). Small pools of purified Memory B cells were seeded into 384-well plates on irradiated CD40L-expressing feeder cells for differentiation into plasma cells as described previously (Liebig, T. M., Fiedler, A., Zoghi, S., Shimabukuro-vornhagen, A. & Bergwelt-baildon, M. S. Von. Generation of Human CD40-activated B cells. J. Vis. Exp. 1373 (2009). doi:10.3791/1373). After 7 days of culture, supernatants from B cell pools were screened for binding activity on SARS-CoV-2 Spike by ELISA. Antibody Heavy and Light Chain variable regions were cloned from positive wells by PCR (Collibri™ Stranded RNA Library Prep Kit for Illumina™ Systems) and whole human IgG reconstructed as described previously (Gu, Y. et al. Defining the structural basis for human alloantibody binding to human leukocyte antigen allele HLA-A*11:01. Nat. Commun. 10, 893 (2019)). Confirmation of binding specificity of cloned human monoclonal antibodies was confirmed by ELISA.

Protein Expression and Purification. The soluble extracellular fragment of human ACE2 (residues 19-615; GenBank: AB046569.1) was cloned into a modified pHLSec (Aricescu, A. R., Lu, W. & Jones, E. Y. A time- and cost-efficient system for high-level protein production in mammalian cells. Acta Crystallogr. D Biol. Crystallogr. 62, 1243-50 (2006)) mammalian expression vector following an N-terminal monoFc, hexahistidine tag and Tobacco Etch Virus (TEV) protease cleavages site. SARS-CoV2-Spike RBD (Dalvie, N. C. et al. Engineered SARS-CoV-2 receptor binding domain improves immunogenicity in mice and elicits protective immunity in hamsters. bioRxiv 2021.03.03.433558 (2021)) fused to CBD (residues 276-434 of Hungateiclostridium thermocellum CipA) was cloned into the pHLmMBP-10 vector (Bokhove, M. et al. Easy mammalian expression and crystallography of maltose-binding protein-fused human proteins. J. Struct. Biol. 194, 1-7 (2016)) (a gift of Luca Jovine; Addgene plasmid 72348) which encodes an N-terminal octahistidine tag, codon-optimized maltose-binding protein (MBP) tag and a TEV site. The coding sequence for the single-chain variable fragment (scFv) of the anti-SARS-CoV CR3022 (Meulen, J. et al. Human monoclonal antibody combination against SARS coronavirus: Synergy and coverage of escape mutants. PLoS Med. 3, e237 (2006)), and was subcloned into pHLmMBP-10 to generate an MBP-scFv fusion construct. Verified plasmids were transfected into Expi293F cells by using the Expifectamine293 transfection kit (ThermoFisher Scientific, #A1435101) to express the secreted proteins following the supplier's standard protocol. Cells were harvested by centrifugation after 6 days of transfection and the supernatants were collected for protein purification. The media were conditioned for Ni-NTA binding by adding 2.5 mL of conditioning buffer, 200 mM HEPES pH 7.5, 3 M NaCl and 10% glycerol; 104 mammalian protease inhibitor cocktail (Nacalai Tesque, #25955-11) per 50 mL media. Proteins were first purified by affinity chromatography using Ni-NTA cartridges (Qiagen, #1046323), followed by size exclusion chromatography by using HiLoad 16/60 Sephadex 200 (Cytiva, formerly GE Healthcare) in gel filtration buffer (20 mM HEPES pH 7.5, 300 mM NaCl, 10% glycerol). To avoid protein crosslinking and aggregation, pooled fractions were supplemented with 0.5 mM TCEP before being concentrated by using Vivaspin centrifugal concentrators (Cytiva). The His-MBP tag of sRBD-CBD was cleaved off by using TEV protease (a gift of NTU Protein Production Platform, proteins.sbs.ntu.edu.sg) at 4° C. overnight with 1:40 mass ratio. Untagged sRBD-CBD was separated from His-tagged proteins by passing the reaction mixture through HisPur-Ni-NTA resin (Thermo Scientific, #88222) pre-equilibrated in 20 mM HEPES pH 7.5, 300 mM NaCl, 10 mM Imidazole. The purified sRBD-CBD sample was buffer exchanged and concentrated in 20 HEPES pH 7.5, 300 mM NaCl, 10% glycerol and 0.5 mM TCEP for storage.

SARS-CoV-2 N protein (residues 1-419; GenBank: YP_009724397.2) was synthesized by Genewiz (USA) and cloned into pET28b(+) bacterial expression vector following a hexahistidine tag and a thrombin cleavage site. Constructed plasmid was transformed into BL21 (DE3) competent cell for protein expression, briefly, culture in LB broth miller (1^{st} Base, #BIO-4000-1 kg) supplemented kanamycin (GOLDBIO, #K-120-25) was allowed to grow till OD₆₀₀ of 0.8 prior it was induced using IPTG (isopropyl β-D-1-thiogalactopyranoside) at final concentration of 0.5 mM for overnight at 16° C. Bacterial cell pellet was then lysed in lysis buffer (20 mM Tris-HCl pH 7.9, 500 mM NaCl) supplemented with protease inhibitor cocktail (Nacalai Tesque, #04080-11) by sonication. Soluble portion was collected and incubated with HisPur-Ni-NTA resin for metal affinity purification. Size exclusion chromatography with HiLoad 16/60 Superdex 75 was carried out for final purification of SARS-CoV-2 N protein with gel filtration buffer (1×PBS pH7.9). Collected protein fractions were pooled and concentrated with Vivaspin centrifugal concentrators prior storage at −80° C.

Biotinylation of monoFc-ACE2. Chemical biotinylation of monoFc-ACE2 was carried out by using EZ-link Sulfo-NHS-LC-Biotinylation kit (ThermoFisher, #21435). Protein was incubated with 20 molar excess of Sulfo-NHS-LC biotin at 4° C. for 2 hrs. The level of biotinylation was measured by HABA assay provided from the kit.

Antibody Profiling by ELISA. SARS-CoV-2 Spike protein, MBP-RBD protein, or nucleocapsid protein was coated on 96-well flat-bottom maxi-binding immunoplate (SPL Life Sciences, #32296) at 7.5 nM, 27 nM, or 40 nM respectively, 100 μL/well at 4° C. overnight. Plate was washed three times in PBS and blocked for 2 hours with blocking buffer: 4% skim milk in PBS with 0.05% Tween 20 (PBST) at 350 μL/well. After three washes in PBST, 100 μL of 80 times diluted plasma samples were added to each well for 1 hour incubation. Plate was then washed three times in PBST and 100 μL of 5000 times diluted goat anti-human IgG-HRP (Invitrogen, #31413), or 5000 times diluted F(ab′)2 anti-human IgA-HRP (Invitrogen, #A24458), or 7500 times diluted goat anti-human IgM-HRP (Invitrogen, #31415) was added to each well for 1 hour incubation protected from light. After three times of plate wash in PBST, 100 μL of 1-Step Ultra TMB-ELISA (Thermo Scientific, #34029) was added to each well. After 3 min incubation in dark, reaction was stopped with 100 μL of 1 M H₂SO₄ and OD₄₅₀ was measured using microplate reader (Tecan Sunrise). OD₄₅₀ reported was calculated by subtracting the background signal from plasma binding to the blocking buffer.

Bio-layer Interferometry (BLI). The N-terminally biotinylated monoFc-ACE2 interaction with RBD-CBD was measured on an 8-channel Octet RED96e system (Forte Bio) with streptavidin biosensor tips (Sartorius). These tips were pre-incubated with assay buffer: PBS, 0.2% BSA and 0.05% Tween 20 for 10 min at 25° C. Then, they were coated with biotinylated mFc-ACE2 to yield a loading thickness of 0.9 nm. After washing the tips with assay buffer, the binding with RBD-CBD was measured in real time by recording the increase in optical thickness of the tips during 600s of association phase. The tips were transfer back into assay buffer during dissociation phase. A two-fold dilution series of RBD-CBD ranging from 6.25 to 100 nM was used. For negative control, the concentration of N-protein was kept at 100 nM for comparison with highest concentration of RBD-CBD. The data was processed by Octet Data Analysis software then transferred into GraphPad Prism 9 for association-dissociation non-linear regression model curve-fitting.

SARS-CoV2 pseudotyped lentivirus production. A third-generation lentivirus system, was used to produce pseduotyped viral particles expressing SARS-CoV2 S proteins via reverse transfection. 36×10⁶ HEK293T cells were transfected with 27 μg pMDLg/pRRE (Addgene, #12251), 13.5 μg pRSV-Rev (Addgene, #12253), 27 μg pTT5LnX-WHCoV-St19 (SARS-CoV2 Spike) and 54 μg pHIV-Luc-ZsGreen (Addgene, #39196) using Lipofectamine 3000 transfection reagent (Invitrogen, #L3000-150) and cultured in a 37° C., 5% CO₂ incubator for 3 days. The viral supernatant was then, harvested and filtered through a 0.45 μm filter unit (Merck). The filtered pseudovirus supernatant was concentrated using 40% PEG 6000 by centrifugation at 1600 g for 60 minutes at 4° C. Lenti-X p24 rapid titer kit (Takara Bio, #632200) was used to quantify the viral titres, as per manufacturer's protocol.

Pseudovirus neutralization assay (pVNT). The ACE2 stably expressed CHO cells were seeded at a density of 5×10⁴ cells in 1004 of complete medium [DMEM/high glucose with sodium pyruvate (Gibco, #10569010), supplemented with 10% FBS (Hyclone, #SV301160.03), 10% MEM Non-essential amino acids (Gibco, #1110050), 10% geneticin (Gibco, #10131035) and 10% penicillin/streptomycin (Gibco, #15400054)] in 96-well white flat-clear bottom plates (Corning, #353377). Cells were cultured in 37° C. with humidified atmosphere at 5% CO₂ for one day. Patient plasma samples were diluted to a final dilution factor of 80 with PBS. The pseudovirus is diluted to a final concentration of 2×10⁶ PFU/ml. In 25 μl there will be 50,000 lentiviral particles. The diluted samples were incubated with an equal volume of pseudovirus to achieve a total volume of 50 μL, at 37° C. for 1 h. The pseudovirus-plasma mixture was added to the CHO-ACE2 monolayer cells and left incubated for 1 h to allow pseudotyped viral infection. Subsequently, 150 μL of complete medium was added to each well for a further incubation of 48 h. The cells were washed twice with sterile PBS. 100 μL of ONE-Glo™ EX luciferase assay reagent (Promega, #E8130) was added to each well and the luminescence values were read on the Tecan Spark 100M. The percentage neutralization was calculated as follows:

$$Neutralization \% = \frac{Readout ( unknown ) - Readout ( infected control )}{Readout ( uninfected control ) - Readout ( infected control )} * 100 \%$$

Modified ELISA-based sVNT. ACE2-Fc was conjugated to peroxidase using Peroxidase Labeling Kit—NH₂ (Abnova, #KA0014) according to manufacturer's protocol. Each well of 96-well flat-bottom maxi-binding immunoplate was coated with 100 μL of 13 nM MBP-RBD at 4° C. overnight. Plate was washed and blocked as described above. Plate was washed three times in PBST and incubated for 1 h with 100 μL/well of plasma samples diluted ten times in blocking buffer. No inhibitor control wells were incubated with blocking buffer. Positive and negative control wells were established by incubating with functionally characterized recombinant monoclonal antibodies targeting SARS-CoV-2 RBD. A characterized neutralizer was included as the positive control and a non-neutralizing binder was included as the negative control. Both monoclonal antibodies were tested at concentrations from 64 nM to 0.5 nM, prepared via 2×serial dilution in the blocking buffer. Subsequently, plate was washed three times and incubated for 1 hour with 0.4 nM ACE2-Fc-peroxidase, 100 μL/well, protected from light. The following steps of color development and absorbance measurement were performed as described above. Inhibition % was calculated as

$$Inhibition \% = ( \frac{OD 450 of negative control ( no inhibition ) - OD 450 of sample}{OD 450 of negative control ( no inhibition )} ) * 100 \%$$

cpVNT assay. Cellulose test strips were prepared using Whatman No. 1 chromatography paper (GE healthcare, #3001-861). The papers were printed with wax-ink printer (Xerox ColorQube 8570, Xerox, USA) to define liquid flow path and testing region. The non-testing regions were printed with the wax ink whereas the testing region were left unprinted. Circular testing region with diameters of 5 mm and 6 mm were prepared. The printed papers were baked at 150° C. for 1 min to allow the wax ink to diffuse through the paper forming hydrophobic boundary throughout the paper thickness. The wax-free testing regions were blocked with 10 μL of 5% BSA in PBS. After air-drying, the test strips were stacking into 3 layers with the 5 mm strips on the topmost layers and 6 mm strips on the second and third layers. The three layered of wax printed paper allows consistent flow of liquid at ˜10 sec when 40 μL of liquid are applied. One piece of Kimwipes paper (11.4 cm×21.6 cm, Kimberly-Clark Professional, #34155) folded in half for 6 times was used as absorbent pad. The three-layered test strips were stacked on top of the folded Kimwipes. All layers were secured together using two paper binders.

nM RBD-CBD in 1% BSA in PBS was prepared and assigned as reagent “A”. 10 nM biotinylated monoFc-ACE2 with 6 nM SA-HRP (Biolegend, #405210) in 1% BSA in PBS was prepared and assigned as reagent “B”. To perform the test, one part of reagent A and one part of reagent B were mixed with two parts of plasma samples, i.e., for one reaction, the mixture contains 10 μL of A, 10 μL of B and 20 μL of sample. The mixture was incubated for 5 minutes at room temperature. 40 μL of the mixture was applied to the testing region. Once sample was fully absorbed the test was washed once with 40 μL of PBS, followed by 40 μL of TMB/H₂O₂ solution (Merck, #T0440). Signals were allowed to develop for 3 min. Images were taken using Xiaomi Redmi A9 phone in a light box equipped with LED lights and save as .jpg format. Images were transferred to a PC. and analyzed using the opened source ImageJ software from NIH. Images were converted from RGB color space to CMYK. Cyan intensity in the testing regions were analyzed. Inhibition % was calculated using the following formula:

$$Inhibition \% = ( 1 - \frac{Cyan intensity of sample}{Cyan intensity of negative control ( no NAb )} ) * 100 \%$$

Pearson's correlation Pearson's correlation coefficiency was calculated using Microsoft Excel function PEARSON.

Calculation of test performance. Disease prevalence was calculated from the sample size. It may not represent the true prevalence. Calculations of each parameter of test performance were done using the following formula:

$$Sensitivity = \frac{True positive}{True positive + False Negative}$$

$$Specificity = \frac{True negative}{False positive + True negative}$$

$$Positive predictive value ( PPV ) = \frac{S e n s i t i v i t y * p r e v a l e n c e}{( ( s e n s i t i v i t y * p r e v a l e n c e ) ) + ( ( 1 - s p e c i v i c i t y ) * ( 1 - p r e v a l e n c e ) )}$$

$$Negative predictive value ( NPV ) = \frac{S p e c i f i c i t y * ( 1 - p r e v a l e n c e )}{( ( 1 - s e n s i t i v i t y ) * p r e v a l e n c e ) ) + ( ( s p e c i f i c i t y * ( 1 - p r e v a l e n c e ) )}$$

$$Accuracy = ( Sensitivity * Prevalence ) + ( Specificity * ( 1 - Prevalence ) )$$

### Example 4—an Antibody-Free Cellulose-Based Vertical Flow Assay for COVID-19 Surveillance

At the end of 2019, emergence of an unusual pneumonia in Wuhan, Hubei province, China was reported. It was later discovered to be caused by infection with the virus SARS-CoV-2 (Severe Acute Respiratory Syndrome Coronavirus-2), which led to the COVID-19 pandemic. COVID-19 is the largest pandemic in the modern era. It has affected more than 200 million people and claimed more than 4 million lives worldwide. Though the fatality rate of SARS-CoV-2 infection is lower than its relatives, SARS-CoV (Severe Acute Respiratory Syndrome Coronavirus) and MERS-CoV (Middle East Respiratory Syndrome Coronavirus), it has high infectivity and transmissibility. Furthermore, emerging of new viral variants have imposed higher risk of infection due to the increase viral infectivity and replication rates. Vaccination programs against SARS-CoV-2 have been initiated world-wide in the late 2020. Although the vaccines provide potential promise to help limit the viral infection and spreading, recent reports revealed that infection can still occur post vaccination. Therefore, to effectively control the pandemic without closing all social activities, frequent surveillance of SARS-CoV-2 infection remains a valuable mean to timely isolate infected individuals. As such, early diagnostic and surveillance of SARS-CoV-2 infection is essential and continues to be urged.

Currently, there are two types of diagnostic tests available for detecting active SARS-CoV-2 infection: 1) molecular-based tests for detecting viral genomic material and 2) antigen rapid tests (ARTs) for detecting viral proteins predominantly spike protein (S protein) and nucleocapsid protein (N protein). Though molecular-based tests are known for their high accuracy and sensitivity, they are not suitable for frequent surveillance applications as lab settings and equipment are needed. Additionally, almost all molecular-based tests are approved for nasopharyngeal swab samples collected by trained personnel, a semi-invasive method that frequently leads to unpleasant experience.

Antigen rapid tests (ARTs) can be deployed as Point-of-Care (PoC) tests and can be used for frequent surveillance. Although ARTs may not have the same high level of sensitivity as the molecular-based tests, the ease of ART workflow allows for high frequency testing. It is suggested that test frequency and rapid turn-around time are more critical than the test sensitivity for COVID-19 surveillance. The larger number of individuals being screened, the more infected yet infectious patients can be identified. Positive results from ARTs suggest relatively high viral loads, hence highly transmissible virus. Therefore, the rapid identification of infected patients via ARTs provides a critical and rapid means to effectively and timely isolate individuals with high transmission potency. For this reason, high attention is drawn to ARTs for surveillance of SARS-CoV-2 infection. Many of the approved ARTs are constrained to nasopharyngeal or nasal swab specimen as the sample matrix. Despite the easy deployment and simple operating procedure, the unpleasant experience of nasopharyngeal and nasal swabs is not appealing to individuals and hence, there may be less motivation to adopt ARTs for frequent testing. In addition, most of the commercial ARTs available use the lateral flow assay (LFA) platform and hence the raw materials required for making of LFAs are in high demand globally. The high demand for LFA production leads to global supply shortage on one of the key LFA components, nitrocellulose paper. Therefore, an alternative system to traditional LFAs for antigen detection would be a great complementary test for surveillance of active SARS-CoV-2 infections.

A new method/system for detecting SARS-CoV-2 antigen to detect COVID-19 is described here. The example describes the detection of SARS-CoV-2 N (Nucleocapsid) protein using saliva as a testing matrix. Engineered scaffold binder proteins rcSso7d are used as capture and reporter reagents to detect SARS-CoV-2 nucleocapsid (N) protein. The binders are engineered using the RAPIDS process. A complementary pair of rcSso7d binders are engineered to bind to the N protein at two different epitopes. One of the binders is fused to CBD (Sso.E2-CBD) and used as a capture reagent. The counterpart binder is tagged to a spacer protein maltose binding protein (MBP) and BA (BA-MBP-Sso.E1) to allow the protein to interact with various types of reporting molecule via the BA-SA interaction. Similar to Example 3, SA-HRP is used as a reporting molecule. A complex of BA-MBP.Sso.E1/SA-HRP is used as the reporter reagent.

Saliva is used as a sample matrix due to its ease of collection. Usage of saliva offers a less invasive sample collection approach as compared to nasal and nasopharyngeal swab. To reduce viscosity of the sample, saliva may be filtered through a 5 μm filter unit prior to running the assay. In addition, because the target protein (N protein) is enclosed inside the virus, the sample is treated with detergent (1% triton X-100) to break the viral membrane and release the N protein. This filtered and detergent-treated saliva matrix is used for the rapid SARS-CoV-2 antigen assay development.

The test is performed by incubating the prepared saliva samples with capture (Sso.E2-CBD) and reporter (BA-MBP-Sso.E1/SA-HPR) reagents for 1 minute. The reaction is then applied onto the cellulose test strip. A washing step is introduced to minimize non-specific binding on the cellulose surface. Finally, TMB substrate is applied to generate colorimetric signal. The colorimetric reaction is left to develop for 3 minutes before the signal is measured. In this example, cyan intensity is used to measure the blue colorimetric signal from HRP/TMP reaction on the cellulose surface. Results showed that when there was an increase in recombinant N protein or SARS-CoV-2 concentration, the cyan intensities increased proportionally, indicating that the test successfully detected the SARS-CoV-2 biomarker and can be used to diagnose COVID-19. Cross-reactivity study showed that the test produced no cross-reactivity to flu-causing pathogens spiked in saliva. These results suggest that the test is highly specific to SARS-CoV-2 (see Results section below for more details).

Advantageously, embodiments of the test can be easily implemented on children and adults for ‘painless’ sample collection, and is thus suitable for frequent testing of SARS-CoV-2 infection for all age ranges. Embodiments of the test provides a short turn-around-time of 5-10 min, up to 4 times shorter than the currently available ARTs which require up to 15-20 min. Using the unique protein engineering technology, the test is designed to be compatible with cellulose paper, an alternative, cost-effective and readily available material. Usage of cellulose paper avoids the risk of nitrocellulose supply chain shortage, an issue that currently hampers the production of many LFA tests. In addition, thermostable rcSso7d proteins are used instead of antibodies for capture of SARS-CoV-2 N protein. Therefore, the reagents may be produced using bacterial system, which is a more cost-effective method as compared to the mammalian cell line system required for antibody production. In addition, a unique vertical flow assay (VFA) with short flow path is developed as an alternative approach to LFA.

### Results

Generation of Affinity Paired rcSso7d Binders Targeting SARS-CoV-2 N Protein

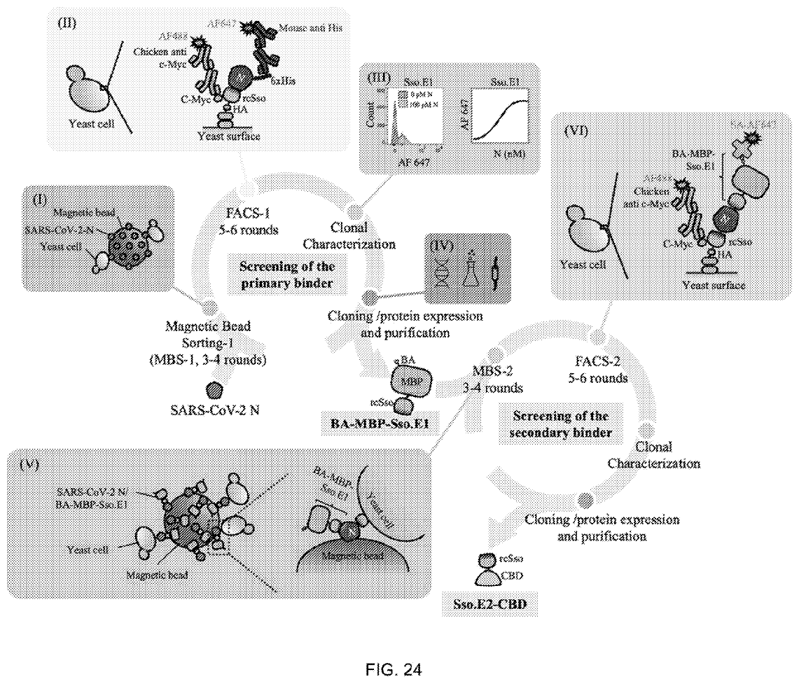

To develop the rapid vertical flow test for detecting SARS-CoV-2 antigen from saliva, a pair of rcSso7d binders binding to different epitopes of N protein were engineered. The RAPIDS screening process (FIG. 24) was applied for discovery of binder pairs from the naïve yeast library that contains 1.4×10⁹ of thermostable rcSso7d variants. Detailed screening of the rcSso7d binding pair was carried out. In brief, recombinant SARS-CoV-2 N protein was used as the target for primary rcSso7d binder screening. The N protein immobilized on magnetic beads were introduced to the naïve yeast library whereby each yeast cell displayed only one variant of rcSso7d on its surface (FIG. 24, step (I)). Following 3-4 rounds of positive (beads labelled with N protein) and negative (bare beads) magnetic bead sorting (MBS), the selected yeast clones were subjected to a more stringent screening using fluorescence-activated cell sorting (FACS). For FACS sorting, the sub-library of the MBS yeast cells was incubated with His(6)-tagged N protein. Each rcSso7d variant was stained with anti c-Myc/AF488 and N protein was stained with anti-His/AF647 (FIG. 24, step (II)). Using AF488 and AF647 markers for FACS, yeast cells which bound strongly to N protein were selected for subsequent round of FACS sorting, whereby N protein concentration was reduced at every subsequent round to ensure selection of high affinity clones. Following 5-6 rounds of FACS sorting, the primary clone, Sso.E1, was selected and characterized (FIG. 24, step (III)). The Sso.E1 was fused to a spacer maltose binding protein (MBP), and a biotin (BA), and referred as BA-MBP-Sso.E1 (FIG. 24, step (IV)). Tagging of BA-MBP improves protein stability and allows biotin-mediated labeling or immobilization. For screening of the secondary rcSso7d binder, a complex of N protein and BA-MBP-Sso.E1 binder was used as the target for the screening (FIG. 24, step (V)). This ensured that the primary binding epitope for Sso.E1 on N protein was blocked, and the selected secondary rcSso7d binders bound to an alternate epitope, thus forming a complementary affinity pair with Sso.E1. A series of MBS (FIG. 24, step (V)) and FACS sorting (FIG. 24, step (VI)) were performed in a similar fashion to the screening of the first binder. For FACS sorting, streptavidin (SA) conjugated with AF647 (SA-AF647) was used for labelling of the rcSso7d BA-MBP-Sso.E1 instead of anti-His/AF647 used in the screening of primary binder rounds (FIG. 24, step (VI)). Finally, the secondary binder, Sso.E2, was selected. The Sso.E2 was fused to cellulose binding domain (CBD), and referred as Sso.E2-CBD, which allows the binder to interact to the cellulose paper. This unique property of CBD facilitates rapid turn-around-time assay development in the subsequent steps.

Through the RAPID screening process, rcSso7d binder pair which bind complementarily to different epitopes of SARS-CoV-2 N protein was successfully engineered. These binders are (i) BA-MBP-Sso.E1 and (ii) Sso.E2-CBD whereby the former will be used as the reporting binder and the latter will be used as the capture binder. The proteins can be easily scaled-up and purified to homogeneity as shown in SDS-PAGE analysis (FIG. 30). Remarkably, this affinity pair of rcSso7d that binds to N protein was successfully identified within 6 weeks.

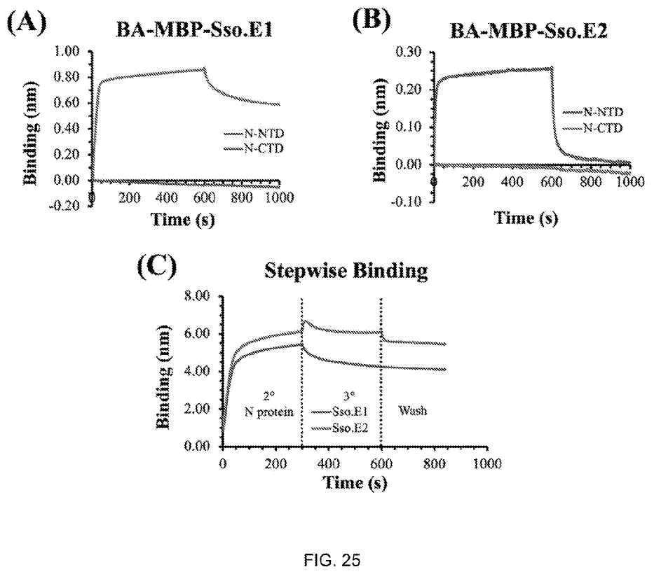

Both Sso.E1 (SEQ ID NO: 4: MATVKFTYQGEEKQVDISKIKIVRRGGQWISFWYDEGGGAYGAGYVSEKDAP KELLQMLEKQ) and Sso.E2 (SEQ ID NO: 5: MATVKFTYQGEEKQVDISKIKNVGRWGQIIDFDYDEGGGAIGIGAVSEKDAPK ELLQMLEKQ) interact with SARS-CoV-2 N protein with excellent binding affinities at the dissociation constants (K_{D}) of 3.2 nM and 4.2 nM, respectively. To determine the binding epitopes of the binder pair, Bio-layer Interferometry (BLI) was performed using truncated N-terminal domain (NTD) and C-terminal domain (CTD) of N protein as ligands. For facile and orientation-specific loading of the Sso binders onto the BLI streptavidin (SA) probes, BA-MBP tag was fused to Sso.E1 and Sso.E2 resulting in BA-MBP-Sso.E1 and BA-MBP-Sso.E2, respectively. BLI results showed that Sso.E1 bound to CTD and Sso.E2 bound to NTD of the N protein, respectively and solely (FIGS. 25A and 25B). N protein is known to form dimers via its CTD. Therefore, results obtained from BLI suggested that the binder, particularly the Sso.E1, capture the tertiary form of N protein via the CTD. To further confirm that the binder pair capture SARS-CoV-2 N protein at different epitopes, stepwise BLI was carried out. BA-MBP-Sso.E1 was loaded on the SA probes. Full length N protein was loaded as a secondary ligand. Sso.E2-CBD or Sso.E1 was loaded as tertiary ligand. Results showed that only the combination of Sso.NP.E1/N protein/Sso.NP.E2 displayed stepwise increase in the binding curve, confirming that the binder pair bind to complementary epitopes on SARS-CoV-2 N protein (FIG. 25C).

### Integration of a Vertical Flow Assay (VFA) on Cellulose Platform

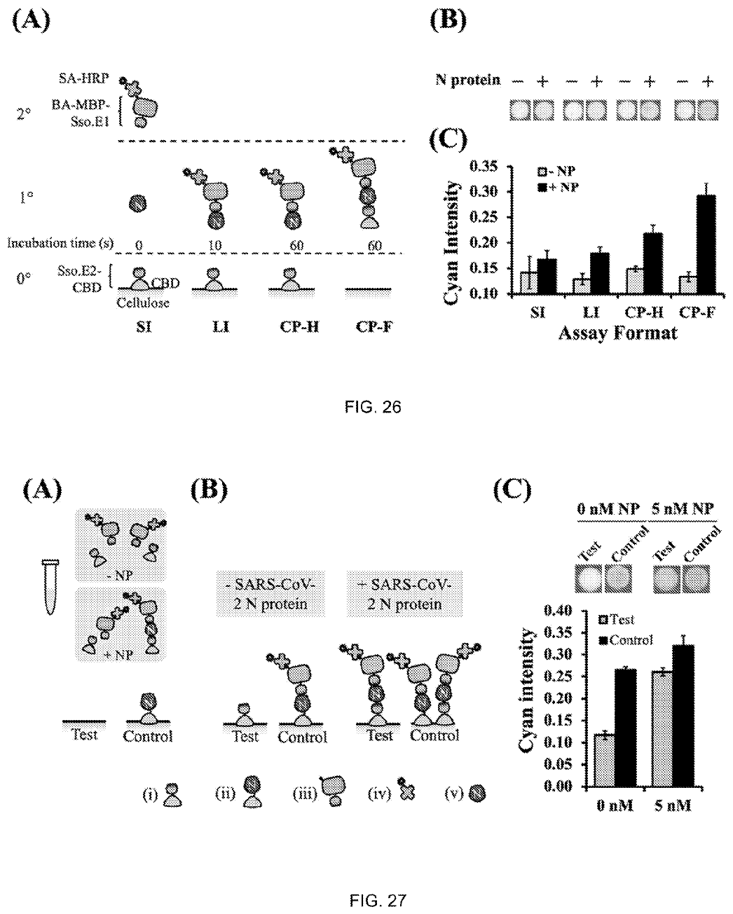

Cellulose paper was selected as the sensor material due to its advantages in price, availability, and high affinity toward CBD. Cellulose is more suitable for the vertical flow assay (VFA) format than the lateral flow assay (LFA) format due to the rather large pore size, for example, as compared to nitrocellulose which is a common material used for LFA. Therefore, a vertical flow assay (VFA) was adopted for development of the rapid SARS-CoV-2 test. To optimize for the VFA, four different assay approaches were tested to determine the workflow that give the best assay performance (FIG. 26). These assay approaches include 1) short incubation (SI) where capture molecule was immobilized on a cellulose surface and analyte and reporting molecules were applied to the cellulose paper in a stepwise manner, 2) limited incubation (LI) where capture molecule was immobilized on a cellulose surface, but analyte and reporter molecules were allowed for a short incubation period of 10 seconds prior loading to the cellulose test matrix, 3) half complex cellulose pulldown (CP-H) where capture molecule was immobilized on a cellulose paper while analyte and reporting molecules were allowed for interaction for a longer period of 1 minute prior loading onto the test matrix and 4) full complex cellulose pulldown (CP-F) where the interaction between capture molecule, analyte and reporting molecule was allowed in an aqueous phase for 1 minute prior loading onto the test matrix (FIG. 26A). All tests were performed in the presence or absence of the analyte (N protein). Application of 3,3′,5,5″-tetramethylbenzidine (TMB) substrate was used to generate colorimetric signals via enzymatic reaction which occurred between TMB and SA-HRP presented on the reporting molecules and had been captured onto the cellulose surface. Cyan intensity was used to measure the test signal. Results revealed that the CP-F test format produced the strongest signal intensity (FIG. 26B) and the highest ‘signal-to-noise’ ratio (+antigen/−antigen) (FIG. 26C). Therefore, the test format 4, CP-F, was chosen for the rapid SARS-COV-2 test development.

For a diagnostic test, a control reaction is required to ensure the active reagent activity and reliable test results, especially when a negative test result (no signal) is observed. A few criteria were considered for constructing of the control reaction including 1) assay reaction shall be able to apply to both test and control reactions to avoid extra steps of sample preparation and 2) control reaction needs to report ‘active’ reagent signal independent of the presence of absence of analyte/antigen in the test sample.

With these considerations, the control reaction was constructed by pre-immobilizing the cellulose control spot with SARS-CoV-2 N protein. To immobilize the N protein on the cellulose surface, the protein was fused to CBD at its C terminus (NP-CBD). The NP-CBD on the cellulose control spot is designed to capture the reporting molecule (BA-MBP-Sso.E1) presented in the sample mixture, followed the CP-F test format (FIGS. 27A and B).

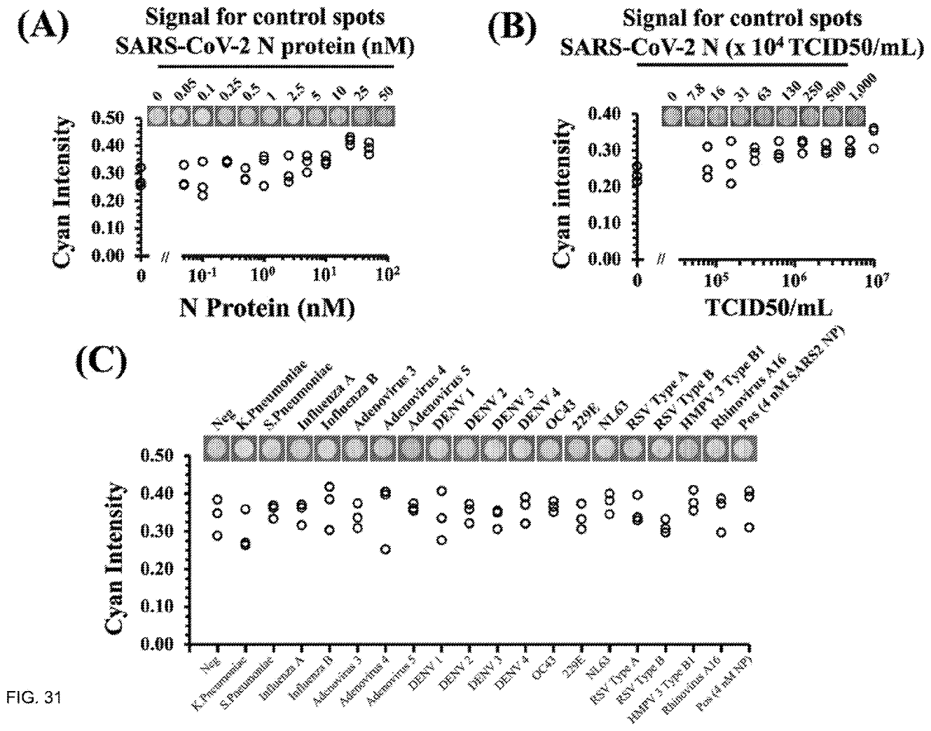

The control spot performance was tested with pooled healthy control saliva samples spiked with either N protein or PBS. Results showed that the control spots produced cyan intensity signals higher than 0.2 for both testing conditions (FIG. 27C). The performance of the control spots was further evaluated under various testing conditions, including different concentrations of N protein spiked in saliva matrix (FIG. 31A), different amount of SARS-CoV-2 virus spiked in saliva matrix (FIG. 31B), and various pathogens spiked in saliva matrix (FIG. 31C). Signals obtained from control spots were observed at comparable level and above the cyan intensity of 0.2 (FIG. 31), indicating that the proposed concept of control spot achieved the desired objective and can be used to monitor the reagent activity for the rapid SARS-CoV-2 VFA.

### Assay Construction and Optimization

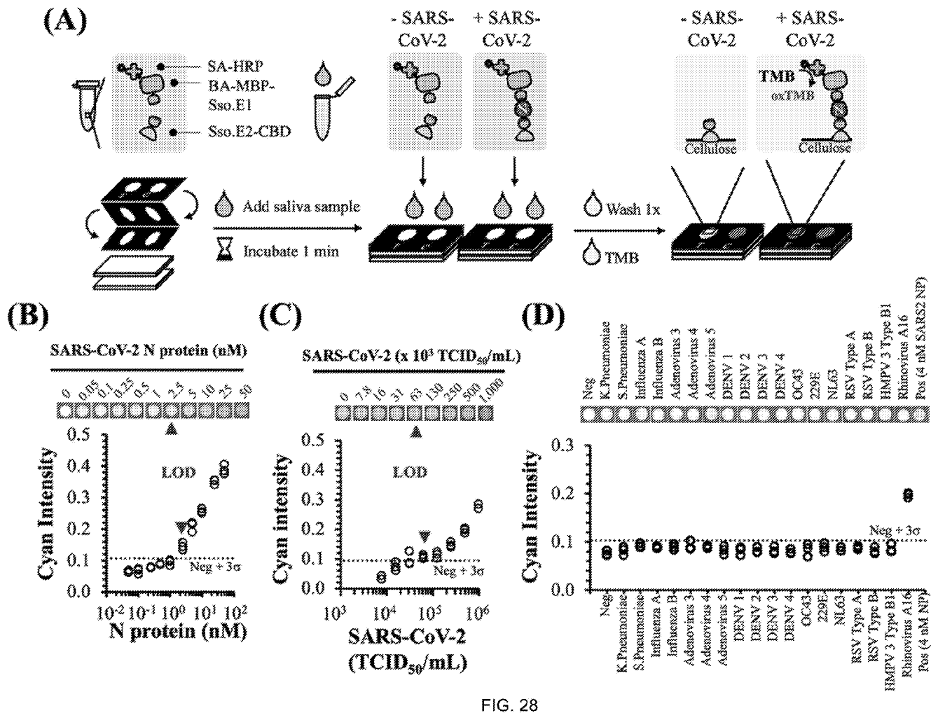

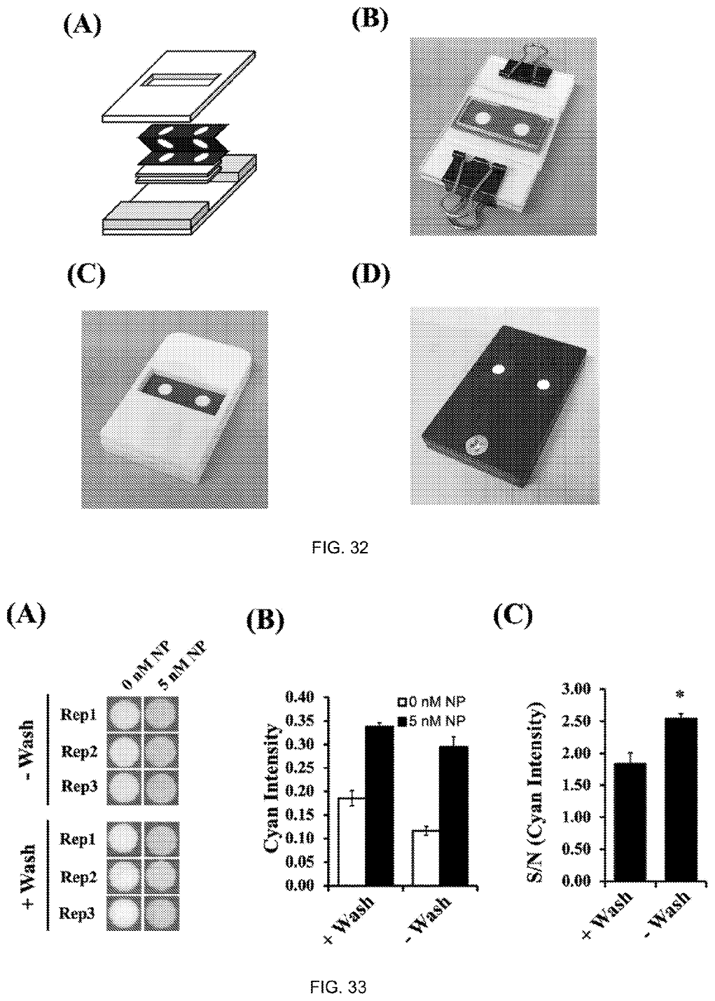

A test strip contains a ‘test’ and a ‘control’ spot (FIG. 28A). The reactive testing regions of both spots were determined using printed hydrophobic ink (FIG. 28A). Diameter of each spot was 4 mm. The test strip was folded in a ‘zig-zag’ manner to construct 3 layered cellulose paper (FIG. 28A). Two pieces of cellulose paper with 1.5 mm thickness were placed underneath the 3-layer folded cellulose paper and used as absorbent pads (FIG. 28A). The folded paper and the absorbent pads were secured together using plastic manifolds and paper binders or double-sided tape (FIG. 32).

Saliva is the desired testing matrix for this assay development. Therefore, pooled saliva from healthy control subjects was used as a testing matrix for the assay optimization. The saliva was filtered through a 5 μm filter to reduce viscosity. N protein is enclosed inside the virus. Therefore, the sample is treated with a detergent to break the viral membrane to allow the release of the N protein. To mimic the final format of the testing sample matrix, the filtered saliva sample used for optimization was treated with 1% v/v triton X-100.

VFA workflow follows the CP-F assay format (FIG. 26). It was observed that the assay produced high background color even in the absence of N protein (FIG. 26B). A washing step was introduced to the assay prior to the colorimetric signal generation using TMB substrate. Results showed that, with the washing step, background signal was substantially reduced and signal-to-noise ratio (+N protein/−N protein) was significantly improved (FIG. 33). Therefore, a washing step was applied to the assay workflow.

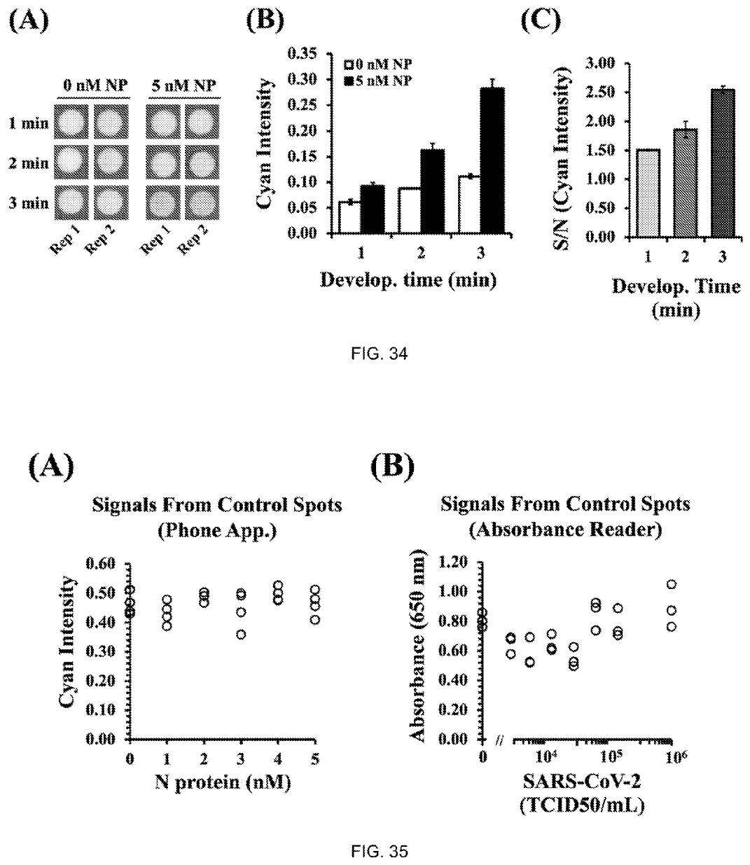

To achieve an even higher signal-to-noise ratio, enzymatic reaction time allowing for optimal colorimetric signal development was evaluated. Using the CP-F workflow with one washing step, the colorimetric reactions were left to develop for different durations following the TMB substrate application. Images of the cellulose test and control spots were taken at different development times and cyan intensities were analyzed. The highest signal-to-noise ratio was achieved with 3 minutes of the color development reaction (FIG. 34). The test was not kept longer than 3 minutes to limit the total assay time to 5 minutes. According to these data, the 3 minutes reaction time was selected for the colorimetric signal development.

To further simplify the assay workflow, testing reagent was prepared as a single tube reaction by mixing capture molecule (Sso.E2-CBD) and reporting molecules (BA-MBP-Sso.E1/SA-HRP) together into a single tube. Based on the assay optimized procedures, the assay workflow can be describe as shown in FIG. 28A. Treated saliva sample was added to the prepared testing reagent and incubated for 1 minute at ambient temperature. 38 μL of the reaction mixture was applied to test spot and another 38 μL to the control spot, respectively. 38 μL of wash solution was applied to the test spot and another 38 μL to the control spot, respectively. 38 μL of TMB substrate was finally applied to the test spot and another 38 μL to the control spot for enzymatic reaction. Cyan intensity was measured after 3 minutes following the color development reaction.

### Assay Performance

With the fully assembled VFA, performance was first tested with different concentrations of SARS-CoV-2 N protein spiked in filtered saliva containing 1% v/v triton X-100 to determine the test limit of detection (LoD). LoD was determined as the lowest concentration that gave signals higher than three times standard deviation values of the background (no antigen) signal (negative+3σ). Results showed that the VFA achieved a LoD at 2.5 nM (FIG. 28B). Corresponding signals from the control spots were shown in FIG. 31. The VFA performance was further evaluated using live SARS-CoV-2. Different concentrations of the virus were spiked in filtered saliva with 1% triton X-100. Results revealed that the test can detect live virus at a concentration as low as 6.3×10⁴ TCID50/mL (FIG. 28C). Corresponding signals from the control spots can be found in FIG. 31. According to viral load quantification studies, the amount of SARS-CoV-2 virus presented in patients is highly variable, ranging from 641 genome copies per mL to 1.34×10¹¹ copies per mL, and the median viral load in sputum is 7.52×10⁵ copies per mL. With the represented LoD sensitivity, the assay can detect patients with the viral load at around the median value, which are those who are most likely to be highly infectious to others.

To further assess the assay performance, the specificity of the rapid SARS-CoV-2 VFA to 2 pneumoniae bacterial strains and 16 viral strains (Table 6) that were spiked into filtered saliva containing 1% triton X-100 was investigated. These pathogens were chosen due to their flu-like symptoms upon infection, including fever and sore throat, symptoms that are also observed in SARS-CoV-2 infection. As shown in FIG. 28D, all 18 pathogens had signal below the cut-off value (negative+3σ), while positive control with 4 nM SARS-CoV-2 N protein showed positive results. Based on these results, it is suggested that the rapid SARS-CoV-2 VFA specificity is 100% against the selected 18 pathogens. Corresponding signal from the control spots are shown in FIG. 31.

TABLE 6
List of non-specific pathogens used for the cross-reactivity study

| | | | | Test | Remarks/expire |
|---|---|---|---|---|---|
| Number | Strain | Source | Lot # | Concentration | date |
| 1 | Human | ATCC VR-3 | 70033218 | 4 × 10⁶ | |
| | adenovirus 3 | | | TCID₅₀/mL | |
| 2 | Human | ATCC VR-5 | 70024114 | 4 × 10⁷ | |
| | adenovirus 5 | | | TCID₅₀/mL | |
| 3 | Coronavirus- | ATCC VR-740 | 70035459 | 4 × 10⁴ | |
| | 229E | | | TCID₅₀/mL | |
| 4 | Coronavirus- | ATCC VR-1558 | 70036255 | 4 × 10⁴ | |
| | OC43 | | | TCID₅₀/mL | |
| 5 | Human | ATCC VR-1572 | 58527797 | 1.25 × 10^{5.5} | |
| | adenovirus 4 | | | TCID₅₀/mL | |
| 6 | Coronavirus | ZEC. | 325222 | 1.17 × 10³ | HI (15 Oct. 2023) |
| | NL63 | 0810228CFHI | | TCID₅₀/mL | |
| 7 | RSV Type A | ZEC. | 324924 | 1.25 × 10⁴ | HI (25 Aug. 2023 |
| | | 0810040ACFHI | | TCID₅₀/mL | |
| 8 | RSV Type B | ZEC. | 323000 | 1.25 × 10⁴ | HI (4 Sep. 2022 |
| | | 0810040CFHI | | TCID₅₀/mL | |
| 9 | HMPV3 Type | ZEC. | 325204 | 0.97 × 10³ | HI (7 Oct. 2023) |
| | B1 | 0810156CFHI | | TCID₅₀/mL | |
| 10 | Rhinovirus | ZEC. | 316699 | 2.5 × 10^{4.1} | HI (N/A) |
| | A16 | 0810285CFHI | | TCID₅₀/mL | |
| 11 | Influenza A | AMR in house | | 3 × 10⁷ | |
| | | culture | | CFU/mL | |
| 12 | Influenza B | AMR in house | | 3 × 10⁷ | |
| | | culture | | CFU/mL | |
| 13 | Dengue 1 | AMR in house | | NA | Doing plaque |
| | | culture | | | assay |
| 14 | Dengue 2 | AMR in house | | 2.5 × 10³ | |
| | | culture | | PFU/mL | |
| 15 | Dengue 3 | AMR in house | | NA | Doing plaque |
| | | culture | | | assay |
| 16 | Dengue 4 | AMR in house | | NA | Doing plaque |
| | | culture | | | assay |
| 17 | Klebsiella | AMR in house | | 4 × 10⁶ | |
| | pneumoniae | culture | | CFU/mL | |
| 18 | Streptococcus | AMR in house | | 3 × 10⁷ | |
| | pneumoniae | culture | | CFU/mL | |

### Translation of the Rapid SARS-CoV-2 VFA to PoC Application

Following the assay optimization and verification, the next step is to translate the rapid SARS-CoV-2 VFA into PoC applications. To do so, there has to be a portable device which could measure cyan intensity signals. Two approaches to translate the VFA to PoC test were tested: 1) adopting of mobile phone camera to capture and interpret the test signals and 2) developing of a portable reader for analysis of the colorimetric signals.

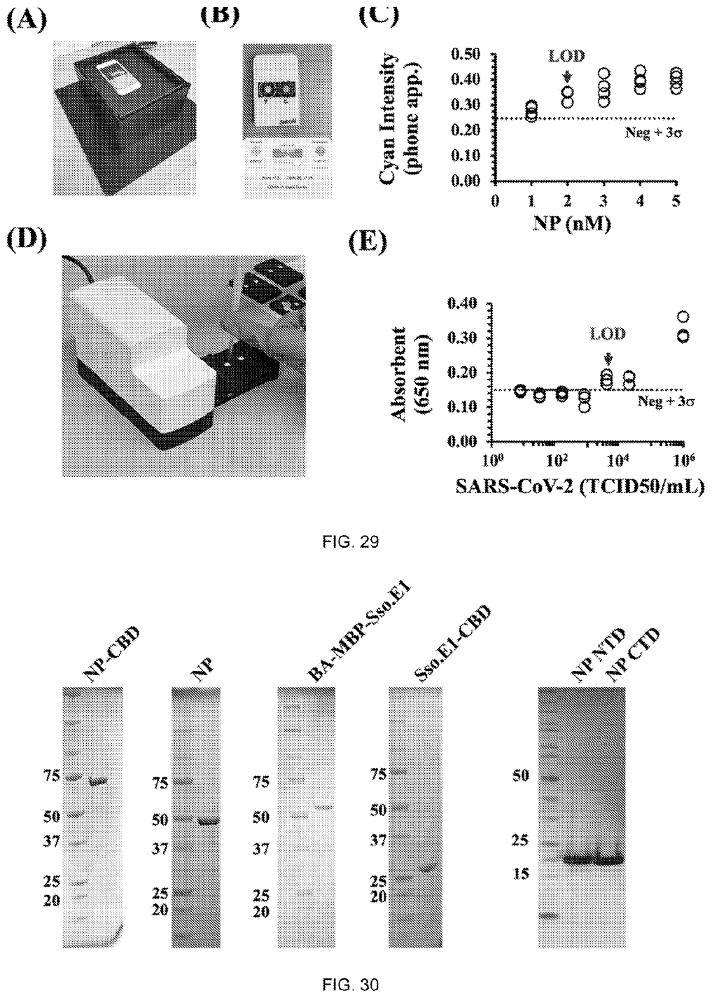

For the first approach, a complementary ‘light box’ was created to ensure the consistency of lighting condition and the phone camera distance (FIG. 29A). A black box dimension 150×230×90 mm³ was equipped with LED light strips at the inner face. The external, top face of the box contains a phone holder to fix the phone position therefore defining a fixed camera distance to all tests measured. A colorimetric signal analysis application was developed for IOS/Android system. The software utilizes phone camera to capture an image, followed by cyan intensity analysis and result interpretation (FIG. 29B). With this approach, LoD of 2 nM SARS-CoV-2 N protein was achieved (FIG. 29C). To the surprise of the inventors, this value outperforms the lab based VFA (FIG. 28B) which achieved LoD of 2.5 nM using Epson scanner and ImageJ software for signal analysis.

For the second approach, a spectrophotometer was designed to measure the red absorbance at 650 nm. The device equipped with a complementary software for signal analysis was outsourced to Attonics Systems Pte. Ltd., Singapore (FIG. 29D). For this approach, the form factor of the cellulose test strips was slightly modified to accommodate the reader system. The test construction remained in a similar fashion to the other experiments performed in this study, in which hydrophobic ink was used to defined hydrophilic boundary and the cellulose paper was folded into 3 layers. Two pieces of 1.5 mm thick cellulose paper was used as absorbent pads. All the pieces were secured together using customized cassette that was produced using aluminum or plastic material. Opening windows were designed above the test and control spots, each with a diameter of 3.8 mm (FIG. 29D). Live SARS-CoV-2 virus spiked in filtered saliva containing 1% v/v triton X-100 was used to assess the performance of the absorbance reader. Results showed that this system achieved LoD of 4.0×10⁴ TCID50/mL, 10 times more sensitive as compared to the LoD obtained from the lab based VFA (FIG. 28C). Based on these two pilot PoC translational approaches, the inventors are confident that the rapid SARS-CoV-2 VFA developed can be practically applied to the PoC settings.

### Discussion

Amidst the available options of SARS-CoV-2 vaccines, the COVID-19 pandemic continues. The importance of early detection and surveillance of SARS-CoV-2 infection has been emphasized and highlighted. Rapid, cheap, and easy-to-use diagnostic tests are still urged and employed as critical tools to help controlling the spreading of the virus.

The inventors have successfully developed a rapid SARS-CoV-2 VFA, on a cellulose platform using unique rcSso7d binder pair, that could detect SARS-CoV-2 N protein from saliva samples. During the lab-based verification, the system achieved LoD of 2.5 nM (FIG. 28B) and 6.3×10⁴ TCID50/mL (FIG. 28C) using recombinant N protein and live virus, respectively, spiked in filtered saliva containing 1% v/v triton X-100. The disclosure has also demonstrated a practicality of translating the developed test to two PoC systems, using the custom designed mobile phone application and the portable absorbance reader device. Results obtained from the PoC systems showed improved test performance in which the tests achieve LoD of 2 nM N protein using the application on the mobile device (FIGS. 29A, B and C) and 4.0×10³ TCID50/mL virus using the custom-made spectrometer system (FIGS. 29D and E). Cross-reactivity challenged against 18 pathogens showed non-specific signal detection, suggesting that the rapid SARS-CoV-2 VFA exhibited 100% specificity against the selected pathogens that cause flu-like symptoms (FIG. 28D).

The group have developed, in parallel, two types of rapid SARS-CoV-2 VFA, one being the ‘bottom readout’ test and the second one being the test described in this example. Building upon the first developed ‘bottom readout’ test where the cellulose test strips required the ‘flipping’ action to apply TMB and for detection the colorimetric signals, the current test simplified the workflow by omitting the ‘flipping’ step. This workflow provides simpler operating steps at PoC settings as well as paves the way for ease of high throughput applications, where the cellulose test strips can be easily treated by different solutions via a liquid handling system.

Saliva has gained much interest as the alternative sample matrix for diagnosis and health monitoring as it provides easy mean of sampling and is non-invasive. Usage of saliva for diagnosing of SARS-CoV-2 infection has been proven feasible by many studies, in which the amount of viral load in saliva was shown to be in a comparable range to the viral load in naso- or oropharyngeal swabs. With an intention to improve the usability of ARTs for SARS-CoV-2 which currently employed nasal or nasopharyngeal swabs as a sample matrix, saliva was selected as a sample matrix due to its ‘painless’ and non-invasive sample collection method. Though the sampling of saliva is easy and straightforward, the sample processing was rather challenging. As the high viscosity of saliva caused by mucous content was problematic for controlling of fluid flow rate. This issue was resolved by filtering of saliva prior to performing the assay. Filtration of saliva may seem trivial during the test development phase, however to implement the test for PoC applications, an easy-to-operate device for saliva filtration at PoC settings has to be considered and sorted.

In conclusion, the inventors have successfully developed a rapid SARS-CoV-2 VFA that detects clinically relevant concentrations of viral N protein present in saliva. Thus far, this test offers the shortest turn-around-time of 5-10 minute from sample to results. Results obtained from this study indicated that the test has a great potential to be practically translated into a PoC diagnostic test for rapid detection of COVID-19. With more frequent testing, the control and isolation of COVID-19 infected individuals could be done more effectively, facilitating safe social interactions within the new community norms.

### Materials and Methods

### Materials

All chemicals were of analytical grades. Sources of standard chemicals that were not listed in the method sections were purchased from Merck/Sigma-Aldrich, Singapore.

### Protein Expression and Purification

All cloning and protein expression work were done in the Escherichia coli DH5α and BL21 (DE3) strains, respectively. N protein NTD and CTD domain boundaries were scouted with the help of NTU Protein Production Platform. Coding sequences were cloned into the pET28b plasmid backbone. For protein expression, relevant plasmids were transformed into E. coli BL21 (DE3). Single colonies were obtained on LB agar plates supplemented with 50 μg/mL kanamycin (Sigma-Aldrich, Singapore) and inoculated in LB media supplemented with 50 μg/mL kanamycin. Each starter culture was grown overnight with shaking at 37° C. and added to 1 L of LB media for protein expression. Each expression culture was allowed to grow at 37° C. with shaking until the OD₆₀₀ reached 0.6-0.9. The culture was then supplemented with 0.5 mM IPTG (Sigma-Aldrich, Singapore) to induce protein production and was grown overnight (16-20 hours) with shaking at 16° C. When expressing the reporting binder BA-MBP-Sso.E1, 0.2 mM of D-biotin (Sigma-Aldrich, Singapore) was also added to the culture prior to IPTG supplementation.

Cell pellet was harvested by centrifugation and subjected to lysis by sonication in lysis buffer (50 mM Tris-HCl, 300 mM NaCl, 10 mM Imidazole, pH 7.6) supplemented with protease inhibitor cocktail (Nacalai Tesque Inc., USA). The lysate was then clarified by high-speed centrifugation at 25,000 g. The soluble fraction (supernatant) was collected and incubated with HisPur Ni-NTA resin (Thermo Fisher Scientific, USA) for metal-affinity purification. Protein-bound resin was washed with buffers containing increasing imidazole concentrations before the desired protein was eluted by using elution buffer containing 300 mM imidazole. All constructs except SARS-CoV-2 N protein were collected at this step. Proteins collected from each fraction were ran on SurePAGE™ precast gels (GenScript, Hongkong, China), and stained using Brilliant Blue R Staining Solution (Sigma-Aldrich, Singapore) for visualization. Fractions containing pure protein of interest were concentrated and buffer-exchanged to PBS with Vivaspin centrifugal concentrators (Sigma-Aldrich, Singapore) and stored at −80° C. SARS-CoV-2 NP was further subjected to size exclusion chromatography with HiLoad 16/60 Superdex 75 (Sigma-Aldrich, Singapore) and eluted with gel filtration buffer. Pure SARS-CoV-2 N protein fractions were pooled and concentrated with Vivaspin centrifugal concentrators and stored at −80° C.

### Processing of Saliva Samples

Pooled saliva was collected from healthy donors by passive drool saliva into collection tubes. To reduce the sample viscosity, collected saliva was filtered with 5 μm syringe filter (Sartorius, USA) and treated with 1 v/v % triton X-100 (Sigma-Aldrich, Singapore). This saliva sample matrix was used for all experiments, otherwise stated.

### Preparation of SARS-CoV-2 Virus Sample

African green monkey kidney cells Vero E6 (CRL-1586; American Type Culture Collection, Manassas, VA, USA) were maintained in Dulbecco's modified Eagle's medium (DMEM; Gibco, Grand Island, NY, USA) supplemented with 8% fetal bovine serum (FBS) and penicillin-streptomycin at 37° C. in 5% CO₂. SARS-CoV-2 (hCoV-19/Singapore/2/2020) virus was previously isolated (Anderson et al., 2020). and the genome sequence is deposited in GISASID (Accession ID EPI_ISL_407987). The virus stock was propagated in Vero E6 cells using DMEM supplemented with 2% FBS. The virus was titrated in ten-fold serial dilutions on 96-well plates of Vero E6 cells to obtain a 50% Tissue Culture Infectious Dose (TCID₅₀). After 4 days incubation, the TCID₅₀ of stock virus was calculated based on eight replicates by the Reed and Muench method (Reed & Muench). The viral stock was pre-diluted in FBS-free DMEM culture media to 107 TCID₅₀/mL. Then, five-fold (2×10⁵, 4×10⁴, 8×10³, 1.6×10³ TCID₅₀/mL) and two-fold dilutions (3.2×10², 1.6×10², 0.8×10², 0.4×10² TCID₅₀/mL) were prepared using DMEM media. Three replicates of SARS-CoV-2 virus samples were prepared at each dilution for the VFA assay.

### Cellulose Test Strips Preparation

Whatman Grade 1 Chr filter paper (GE Healthcare, USA) was used for fabrication of the test strip. Each test strip contained 1 test spot and 1 control spot, each with a reactive circular testing area of 4 mm diameter. To define the reactive testing area that allows liquid to pass through, wax ink was printed (ColorQube 8570, Xerox, USA) on to the cellulose paper to create hydrophobic boundary around the testing areas. The printed paper was baked for 1 minute at 150° C. to allow the wax ink to diffuse throughout the paper thickness, forming continuous hydrophobic boundary throughout the paper layer. The test strip was folded in a zig-zag motion to create a 3 layered test strip, generating the final strip ‘length×width’ dimension of 30×15 mm². The top layer of the wax ink printed test strip was treated with 10 μL of 5% BSA dissolved in PBS. The treated papers were air dried and stored at 4° C. until used.

### Optimization of Different VFA Test Formats

For the test conditions that required immobilization of Sso.E2-CBD on the test strip, the top layered of the test strip was treated with 10 μL of 2 μM Sso.E2-CBD diluted in PBS. To ensure that the proteins were immobilized on the cellulose surface, the cellulose test strip was suspended in the air, allowing the protein to remain on the test strip for ˜2 min, subsequently excess liquid was absorbed away using multi-purpose towel paper. The test strip was blocked with 10 μL 5% BSA in PBS, air dried and stored in 4° C. until used. For the condition which did not require pre-immobilization of Sso.E2-CBD, the cellulose test strip was treated with only 5% BSA. The test strips were constructed following the protocol described in the ‘VFA Test Assembly’.

PBS was used as the ‘sample matrix’ base for this experiment. The sample matrix was prepared by spiking PBS or 5 nM N protein into the PBS mixture reaction. Reporting molecule was prepared by mixing 500 nM of BA-MBP-Sso.E1 with 62.5 pM of streptavidin (SA) horse radish peroxidase (SA-HRP), (Biolegend, USA) in PBS. The reaction was kept incubated for at least 15 min on ice to ensure effective complex formation of BA-MBP-Sso.E1/SA-HRP prior to use. For the condition which did not require Sso.E1-CBD to be pre-immobilized onto the cellulose surface, 500 nM of Sso.E1-CBD was used for the mixture reaction. All mixture reaction volumes were prepared at 40 μL per reaction. This reaction volume maintained the hypothetical equimolar concentration of Sso.E1 in the solution and on the pre-immobilized cellulose surface, which was equivalent to 2×10⁻¹⁸ moles. 40 μL of 3,3′,5,5″-tetramethylbenzidine (TMB) substrate (Sigma-Aldrich, Singapore) was applied to generate colorimetric signal. After 3 minutes of the color development Image was taken using Epson Perfection V750 Pro Scanner (Epson, Singapore). Images were stored as .jpg files for subsequent colorimetric signal analysis.

### VFA Test Assembly

The 3 layered wax ink printed cellulose test paper was prepared according to the protocol described in ‘Cellulose Test Strip Preparation’. 1.5 mm-thick cellulose filter paper (Whatman gel blotting papers grade GB005, Sigma-Aldrich, Singapore) was cut to 30×15 mm² and was used as a material for absorbent pads. Two pieces of the absorbent material were placed underneath the 3 layered wax ink printed paper. To hold all the cellulose test strip components together, plastic manifolds were created using 2 mm thickness acrylic sheet. The acrylic sheet was cut into two pieces using a laser cutter (Epilog Fusion Edge Laser System, USA), each piece with a dimension of 30×50×2 mm³. An opening dimension 20×10 mm² was cut through on one of the acrylic pieces, providing access to the test strips (FIG. 32). 3 mm thickness acrylic sheet was laser cut into two pieces of rectangular cuboid, each with a dimension of 10×30×3 mm³. These two pieces of acrylics were used as spacers that restricted the clamping of acrylic manifolds to 3 mm (FIG. 32), therefore defining consistent pressure applied to different test strips. The acrylic manifolds and the test strip components were held together using paper binders or double-sided tape.

The test strip used for the custom-made spectrophotometer (Attonics Systems, Singapore) was assembled using re-usable cassettes made of aluminum, dimensioned 30×50×3.2 mm³. The cassette contained two circular openings, each with 3.8 mm diameter, for accessing of test and control spots. Following the principle set by the acrylic manifolds, the height in the inner chamber of the cassette was maintained at 3 mm for a consistent pressure applied to the test strip components.

### Image Analysis

Images of all test strips were taken using Epson Perfection V750 Pro Scanner (Epson, Singapore), otherwise stated. All images were saved in .jpg format. Cyan intensity was analyzed from each image using an open-source ImageJ software from The US National Institute of Health (NIH). Image was separated into single channel of CYMK color space. Cyan intensity was calculated based on a mean value of the defined area in an image.

For assessment of live virus performed on the VFA, test result images were taken using an iPhone 5S instead of the scanner. The phone was used instead of the scanner due to restrictions of the BSL3 facility employed to perform the test. Images from the phone were transferred to a PC and analyzed using ImageJ software. The analysis was performed using a similar process applied to images acquired from the scanner. Despite the different image acquisition devices, cyan intensities were found to be within a comparable range.

### Example 5—Rapid Detection of SARS-CoV-2 Neutralizing Antibodies from Non-Processed Whole Blood Samples and its Application on Monitoring of Immunity Status Against SARS-CoV-2 Variants

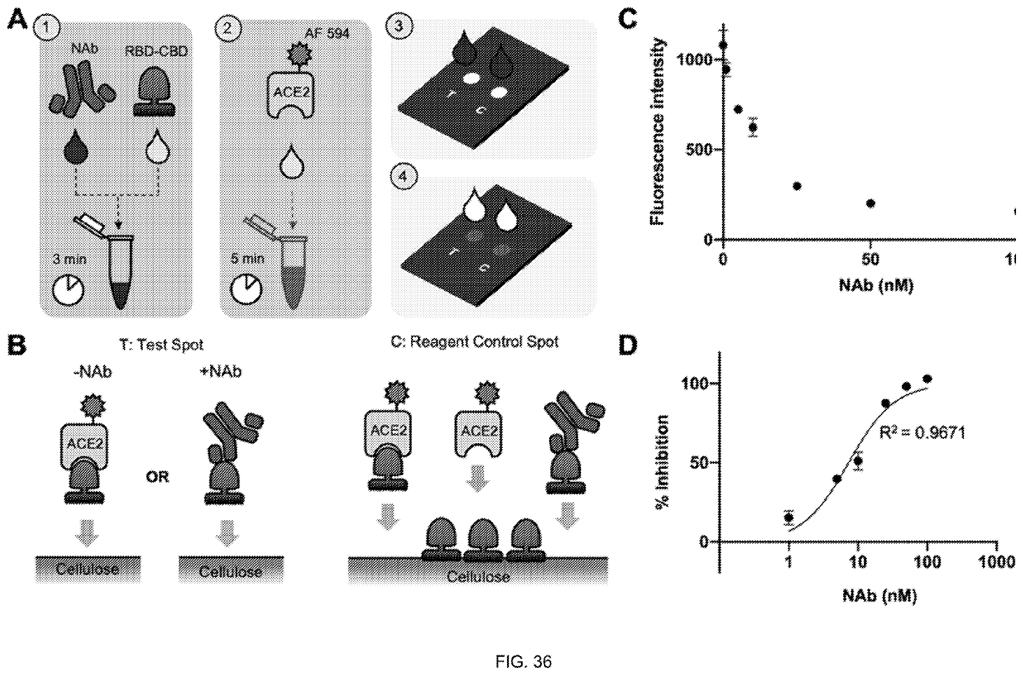

Stemming from the application in Example 3, the inventors further developed a test better suited to Point-of-Care (PoC) applications by using non-processed whole blood samples (fingertip or venous) instead of plasma or serum samples which require sample processing steps in a laboratory. Similar to the application in Example 3, this test utilizes the interaction between RBD/ACE2 to determine the level of NAb. However, HRP cannot be used as a reporting molecule in whole blood sample due to the presence of peroxidase in red blood cells that interferes with the colorimetric signal generation. Instead of using HRP, the ACE2 protein was conjugated to fluorescent molecules (ACE2-FI) i.e., AF594 and used as a reporting reagent (FIG. 36A).

A modified workflow was applied to this assay application to improve the test sensitivity. Whole blood sample was first incubated with RBD-CBD for 3 minutes, subsequently ACE2-FI was added to the reaction and incubated for another 5 minutes (FIG. 36A). This two-step incubation allows NAb to interact with RBD-CBD first before exposing to ACE2-FI, therefore facilitating effective interaction of NAb to RBD-CBD. Following the incubation steps, the reaction was applied to ‘Test’ and ‘Control’ spots on the cellulose-based vertical flow device (FIGS. 36A and B). A washing step was applied to minimize non-specific signals on the cellulose surface before fluorescent intensities were measured.

In a similar fashion to Example 3, absence or low concentrations of NAb led to high fluorescent intensities (FIG. 36C) whereas increase in NAb concentrations resulted in proportional reduction of fluorescent intensities (FIG. 36C). For ease of test result interpretation, inhibition percentage graph was plotted (FIG. 36D), in which the high fluorescent intensity signals obtained from low NAb levels were represented as low % inhibition and vice versa.

Because the positive NAb signals are derived from the absence of fluorescent intensity, a control spot is critical to ensure that the loss of signals are due to presence of NAb and not the malfunction of reagents. A control spot was designed to produce high fluorescent signal regardless of presence or absence of NAb. To do so, high concentration of RBD-CBD was immobilized on the cellulose surface at the control spot to capture ACE2-FI from the sample mixture (FIG. 36B).

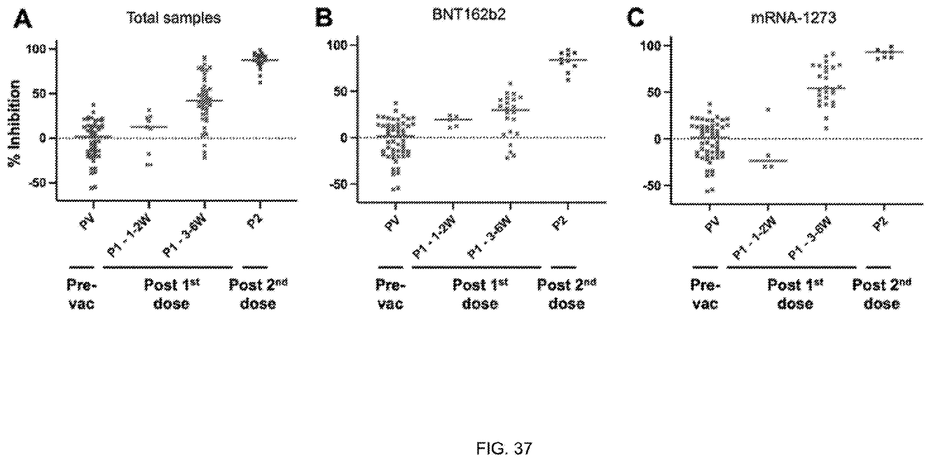

Using this assay workflow, the inventors demonstrated that the test can detect different levels of NAb from samples that present different stages of COVID-19 vaccination (FIG. 37). These results indicate that the rapid cellulose-based SARS-CoV-2 NAb test can distinguish different levels of NAb from different stages of vaccinated subjects, therefore supporting the usability of the test for PoC mass screening of herd immunity and vaccine efficacy.

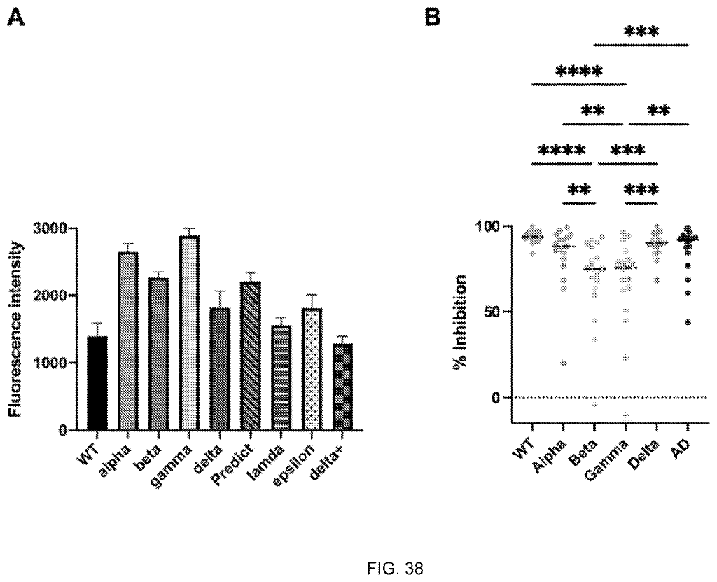

In addition, the inventors have also demonstrated that the rapid cellulose-based SARS-CoV-2 NAb test can be used to monitor immunity status against the SARS-CoV-2 variants. Recombinant RBD proteins from each of the SARS-CoV-2 variant were fused to CBD (RBDv-CBD) and used as capture reagents. Using non-infected and non-vaccinated blood as sample matrix, the results showed that most of the RBD from the variants produced higher fluorescent intensities as compared to the wild-type (WT) SARS-CoV-2 (FIG. 38A). These results indicated that the variants interact with ACE2 more effectively thereby producing higher fluorescent intensities. Blood samples from fully vaccinated samples were tested on these variants. Results showed that, more than 50% inhibitions were still observed from most of the subjects on all variants. These data suggested that majority of the tested vaccinated samples exhibited protective immunity against SARS-CoV-2 variants (FIG. 38B).

### Applications

Embodiments of the method and system disclose herein relates to a cellulose pull down (CP) technology that can capture target analytes more effectively using (i) pre-mixing solution and (ii) cellulose binding domain fused to an analyte binder. Cellulose binding domain (CBD) has a very high affinity toward cellulose substrate. It can be captured on to the cellulose substrate within less than a second. By fusing the analyte binder to CBD, embodiments of the method and system take advantage of the CBD to pull the assay complex down to a cellulose substrate.

In embodiments of the method and system, the sample, the reporter agents and optionally the capture agents are pre-incubated in a solution off site of a cellulose substrate. The pre-incubation step allows excess time for the assay complex (e.g. reporter agent-analyte complex or reporter agent-analyte-capture complex) to form (typically 1-3 min is sufficient). Once the complex is formed, it is applied to the cellulose substrate. The whole assay complex can be instantaneously pulled down to the cellulose paper via high affinity CBD-cellulose interaction, resulting in high sensitivity of signal production. In a scenario where a full assay complex incubation is not favorable, half complex pre-incubation (analytes with reporter reagents) can be applied. The capture agents comprising CBD can be pre-immobilized on to the cellulose substrate prior to performing the assay. The half complex formation during the pre-incubation allows most of the target analytes captured to generate signals, therefore promoting high sensitivity of signal production.

Rapid diagnostic tests often compromise sensitivity for rapid detection time. Embodiments of the method and system disclosed herein generally improve the performances of RDTs to the next level of sensitivity while maintaining the rapid detection time. Embodiments of the cellulose pull down (CP) technology allows sufficient time for full sandwich complex formation and effective capture of the full sandwich complex on to cellulose substrate. As a result, embodiments of the method allow high sensitivity signal detection in a short period of time. In addition, the use of low capture agent concentration could save up to 8 times on raw materials which could further lower down the production cost significantly.

In addition to RTD, embodiments of the method and system can be applied to wide array of biomedical and life science applications including protein separation. Altogether, embodiments of the method have great potential to be expanded to many applications. Examples of such applications include, but are not limited to, the rapid detection of SARS-CoV-2 neutralizing antibodies from human plasma/serum samples, the rapid detection of SAR-CoV-2 antigen from saliva samples and the rapid detection of SARS-CoV-2 neutralizing antibodies from non-processed whole blood samples and its application on monitoring of immunity status against SARS-CoV-2 variants.

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A method of detecting an analyte in a sample, the method comprising:
applying the sample to a cellulose substrate to allow said analyte, if present in said sample, to be captured onto said cellulose substrate by a capture agent comprising a cellulose binding domain (CBD), wherein said capture agent is:
(i) incubated with said sample prior to the applying step; and/or
(ii) immobilised on said cellulose substrate prior to the applying step; and
determining a presence or absence of said analyte captured on said cellulose substrate by said capture agent.

2. The method of claim 1, wherein determining a presence or absence of said analyte captured on the cellulose substrate by said capture agent comprises detecting a signal effected by a reporter agent on the cellulose substrate, wherein said reporter agent:
(i) comprises an analyte binder and said reporter agent is contacted with the sample to allow said reporter agent to bind to said analyte, if present, in said sample; or
(ii) comprises a competing binder having affinity for said capture agent and said reporter agent is contacted with said capture agent to allow said reporter agent to bind to said capture agent.

3. The method of claim 1, wherein the amount of said capture agent is not more than 1000-fold molar excess, optionally not more than 100-fold molar excess, further optionally not more than 60-fold molar excess of said analyte.

4. The method of claim 2, wherein where said reporter agent comprises an analyte binder, the method comprises incubating the reporter agent and the capture agent with said sample prior to the applying step and said analyte, when present, is part of a reporter agent-analyte-capture agent complex prior to the applying step.

5. The method of claim 2, wherein where said reporter agent comprises an analyte binder, the method comprises incubating the reporter agent with said sample and immobilizing said capture agent on the cellulose substrate prior to the applying step and said analyte when present, is bound to the reporter agent to form a reporter agent-analyte complex prior to the applying step.

6. The method of claim 2, wherein where said reporter agent comprises a competing binder, the method comprises incubating said capture agent with said reporter agent and/or said sample prior to the applying step to form a reporter agent-capture agent complex and/or an analyte-capture agent complex prior to the applying step.

7. The method of claim 2, wherein where said reporter agent comprises a competing binder, the method comprises incubating said reporter agent with said sample and immobilizing said capture agent on said cellulose substrate prior to the applying step and dispensing the reporter agent incubated with said sample on said cellulose substrate after the applying step.

8. The method of claim 1, wherein a plurality of capture agents is immobilised on said cellulose substrate in a substantially uniform orientation.

9. The method of claim 1, wherein in said capture agent, said CBD is coupled to a C-terminus of a binding protein for said analyte.

10. The method of claim 1, wherein the presence or absence of said analyte in said sample is determined within 15 minutes from the start of the incubating step.

11. The method of claim 1, wherein said method is a method of detecting coronavirus in a sample.

12. The method of claim 1, wherein said method is a lateral flow assay method.

13. The method of claim 1, wherein said method is a vertical flow assay method and the applying step comprises allowing the sample to flow through a plurality of cellulose substrate layers.

14. The method of claim 11, wherein the analyte is an antibody against SARS-CoV-2, and wherein the method comprises:
applying a sample from the subject to a cellulose substrate to allow said antibody, if present in said sample, to be captured onto said cellulose substrate by a capture agent comprising a cellulose binding domain (CBD), wherein said capture agent is:
(i) incubated with said sample prior to the applying step; and/or
(ii) immobilised on said cellulose substrate prior to the applying step;
contacting the capture agent with a reporter agent to allow said reporter agent to bind to said capture agent, wherein the reporter agent comprises a competing binder having affinity for said capture agent; and
detecting a signal effected by the reporter agent to determine a presence or absence of said antibody captured on said cellulose substrate by said capture agent.

15. The method of claim 14, wherein the sample is selected from the group consisting of: a plasma sample, a serum sample and a whole blood sample.

16. The method of claim 14, wherein said capture agent is incubated with said sample before said capture agent is contacted with said reporter agent.

17. The method of claim 11, wherein the analyte is a SARS-CoV-2 protein, and wherein the method comprises:
incubating a sample obtained from a subject with a reporter agent to allow said reporter agent to bind to a SARS-CoV-2 protein, if present, in said sample;
applying the incubated sample to a cellulose substrate to allow the SARS-CoV-2 protein that is bound to said reporter agent to be captured onto said cellulose substrate by a capture agent comprising a cellulose binding domain (CBD), wherein said capture agent is:
(i) incubated with said sample prior to the applying step; and/or
(ii) immobilised on said cellulose substrate prior to the applying step; and
detecting a signal effected by the reporter agent to determine a presence or absence of said SARS-CoV-2 protein captured on said cellulose substrate by said capture agent.

18. The method of claim 17, wherein the SARS-CoV-2 protein comprises SARS-CoV-2 nucleocapsid (N) protein.

19. The method of claim 17, wherein said reporter agent and said capture agent are configured to bind to different epitopes of the SARS-CoV-2 protein.

20. The method of claim 17, wherein said sample is selected from the group consisting of: a saliva sample, a sputum sample, a nasal fluid sample, a pharyngeal fluid sample, a nasopharyngeal fluid sample and an oropharyngeal fluid sample.


SEQUENCE LISTING (5 sequences)

SEQ ID NO: 1 - protein, 159 aa, Artificial Sequence
  CBD
PVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKEP

SEQ ID NO: 2 - DNA, 477 bp, Artificial Sequence
  CBD
ccggtatcaggcaatttgaaggttgaattctacaacagcaatccttcagatactactaactcaatcaatcctcagttcaaggttactaataccggaagcagtgcaattgatttgtccaaactcacattgagatattattatacagtagacggacagaaagatcagaccttctggtgtgaccatgctgcaataatcggcagtaacggcagctacaacggaattacttcaaatgtaaaaggaacatttgtaaaaatgagttcctcaacaaataacgcagacacctaccttgaaataagctttacaggcggaactcttgaaccgggtgcacatgttcagatacaaggtagatttgcaaagaatgactggagtaactatacacagtcaaatgactactcattcaagtctgcttcacagtttgttgaatgggatcaggtaacagcatacttgaacggtgttcttgtatggggtaaagaaccc

SEQ ID NO: 3 - DNA, 29903 bp, Severe acute respiratory syndrome coronavirus 2
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 4 - protein, 62 aa, Artificial Sequence
  Sso.E1
MATVKFTYQGEEKQVDISKIKIVRRGGQWISFWYDEGGGAYGAGYVSEKDAPKELLQMLEKQ

SEQ ID NO: 5 - protein, 62 aa, Artificial Sequence
  Sso.E2
MATVKFTYQGEEKQVDISKIKNVGRWGQIIDFDYDEGGGAIGIGAVSEKDAPKELLQMLEKQ